(19) United States
(12) Patent Application Publication
(10) Pub. No.: US 2026/0347845 A1
(43) Pub. Date: Nov. 5, 2026

(54) ANTISENSE OLIGONUCLEOTIDES FOR THE TREATMENT OF ATHEROSCLEROTIC CARDIOVASCULAR DISEASE

(71) Applicant: ProQR Therapeutics II B.V., Leiden (NL)

(72) Inventors: Maarten HOLKERS, Leiden (NL); Aliye Seda YILMAZ-ELIS, Leiden (NL); Gerardus Johannes PLATENBURG, Leiden (NL); Bart KLEIN, Leiden (NL); Bruno Filipe Madeira DE ALBUQUERQUE, Leiden (NL); Angela HELFRICHT, Leiden (NL)

(73) Assignee: ProQR Therapeutics II B.V., Leiden (NL)

(21) Appl. No.: 19/158,352

(22) PCT Filed: Feb. 19, 2024
(86) PCT No.: PCT/EP2024/054190

§ 371 Date: Aug. 20, 2025

(30) Foreign Application Priority Data
Feb. 20, 2023 (GB) 2302381.5
May 5, 2023 (GB) 2306669.9

(51) Int. Cl. C12N 15/113; C12N 15/86

(52) U.S. Cl. CPC C12N 15/113; C12N 15/86; C12N 2310/11; C12N 2310/321; C12N 2310/322; C12N 2310/334; C12N 2310/335; C12N 2310/351; C12N 2310/3533; C12N 2750/14143

(57) ABSTRACT

The disclosure relates to the field of cardiovascular diseases, such as atherosclerotic cardiovascular diseases (ASCVD) caused by (high) levels of LDL-C and/or triglycerides in the plasma. The disclosure involves oligonucleotides and the use thereof in RNA editing methods in targeting a target adenosine in an endogenous (preferably wildtype) human ANGPTL3 transcript in a cell, preferably a liver cell, more preferably a hepatocyte, to yield an ANGPTL3 protein that has a diminished or lowered ability to inhibit lipolysis, because of the RNA editing.

### TECHNICAL FIELD

This disclosure relates to the field of medicine. It relates to the field of diseases, such as atherosclerotic cardiovascular disease (ASCVD), that are caused by elevated levels of Low-Density Lipoprotein Cholesterol (LDL-C, also known as “bad” cholesterol) and triglycerides. The disclosure involves the use of nucleotide editing technology in targeting the gene transcript encoding the Angiopoietin-like 3 (ANGPTL3) protein to bring about amino acid changes that yield an ANGPTL3 protein with a reduced ability to inhibit lipolysis.

### BACKGROUND

Elevated plasma levels of LDL-C are well-known risk factors for atherosclerotic cardiovascular disease (ASCVD). The strong causal association between plasma LDL-C and ASCVD forms the basis for the use of aggressive LDL-lowering therapies in individuals at high risk of ASCVD. Effective therapies are available for lowering plasma cholesterol in most people, including statins, selective cholesterol absorption inhibitors, and PCSK9 inhibitors. In contrast, the therapeutic options are more limited for individuals with homozygous familial hypercholesterolemia (HoFH). For these patients, as well as for hypercholesterolemic individuals who are largely refractory to the above treatments, there is a continued need for new treatments that can lower plasma LDL-C levels. Apart from LDL-C, evidence is accumulating that shows that elevated levels of plasma triglycerides are also a risk factor for ASCVD. Considerable attention has been focused on the enzyme lipoprotein lipase (LPL), which catalyses the hydrolysis of plasma triglycerides and is rate limiting for triglyceride uptake into muscle, heart, and adipose tissue. Due to its importance in plasma lipid metabolism, the activity of LPL in different tissues is carefully regulated to be able to cater lipid uptake to local lipid demand. The Angiopoietin-like 3 (ANGPTL3) protein is one of the proteins involved in the regulation of LPL activity. With the approval of Evinacumab, a monoclonal antibody directed against ANGPTL3 as an add-on treatment for patients with HoFH that carry defective LDL receptors, ANGPTL3 has gained widespread recognition as a therapeutic target. ANGPTL3 is a 45 kDa protein that is exclusively produced in hepatocytes. The protein impairs clearance of triglyceride-rich lipoproteins and raises plasma levels of triglycerides by inhibiting the activity of LPL as indicated above. It also raises plasma High-Density Lipoprotein Cholesterol (HDL-C) levels by inhibiting endothelial lipase. The ability of ANGPTL3 to raise plasma triglycerides depends on ANGPTL8. It is released from liver cells as a complex with ANGPTL8, although it is predominantly detected in free form in blood plasma. The physical association with ANGPTL8 greatly increases the affinity of ANGPTL3 for LPL and creates a very potent endocrine inhibitor of plasma triglycerides in heart, skeletal muscle, and brown adipose tissue.

Several strategies, which currently are in different stages of R&D pipelines, have been developed to inactivate ANGPTL3 and improve plasma lipid levels. The above-mentioned Evinacumab (Evkeeza) proved to be very effective in lowering LDL-C(47%), HDL-C(30%) and triglycerides (55%) in HoFH patients. Clear disadvantages of Evinacumab are the required high frequency of injections (approximately every 4 weeks) and costs. The development of a GalNAc conjugated antisense oligonucleotide (a gapmer) that targeted the transcript of ANGPTL3 for specific breakdown and thereby reduction of ANGPTL3 expression (also referred to as Vupanorsen, developed by lonis Pharmaceuticals, Inc. and Pfizer, Inc.) was discontinued after a global phase 2b trial in which it met its primary endpoint and achieving a statistically significant reduction in non-HDL-C, triglycerides, and ANGPTL3 expression, but in which also serious side-effects were observed. Phase 2 (GATEWAY) and phase 2b (ARCHES-2) trials with an siRNA molecule (ARO-ANG3, developed by Arrowhead Pharmaceuticals, Inc.) for the treatment of HoFH patients and patients with elevated levels of LDL-C/triglycerides, respectively, are currently ongoing.

Even though promising results were obtained in Ldlr−/−mice, that showed a reduction of more than 50% in LDL-C and plasma triglycerides levels, genomic targeting using CRISPR-based technologies have the great disadvantage of generating a permanent change in the genome, with the additional possibility of off-site targeting and unrelated DNA changes.

The present disclosure aims to provide one or more alternative, and/or improved, compounds or compositions for targeting the human ANGPTL3 transcript and use thereof in the treatment of diseases related to high levels of plasma LDL-C and triglycerides.

### SUMMARY OF THE INVENTION

Disclosed herein is an antisense oligonucleotide that can yield RNA editing in a cell and that is conducive to ADAR-mediated adenosine deamination and that is capable of forming a double-stranded complex with a region of an endogenous human ANGPTL3 transcript molecule in a cell, wherein the region of the ANGPTL3 transcript molecule comprises a target adenosine, and wherein the double-stranded complex can recruit an endogenous ADAR enzyme to deaminate the target adenosine into an inosine, thereby editing the ANGPTL3 transcript molecule. The antisense oligonucleotide according to the present disclosure that yields (or causes, or produces, or results in) RNA editing is herein generally abbreviated to ‘EON’. Preferably, the ANGPTL3 transcript molecule is a pre-mRNA or an mRNA molecule. In an embodiment, the cell is a human liver cell, preferably a hepatocyte. A preferred target adenosine is the first adenosine of the codon AAG at position 63 in the mature human ANGPTL3 protein encoding lysine (K), which will be edited to IAG (GAG) coding for glutamic acid (E), generating a change that is often herein referred to as K63E. In an embodiment, the EON comprises at least one nucleotide comprising one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. Disclosed is also a vector, preferably a viral vector, more preferably an adeno-associated virus (AAV) vector, comprising a nucleic acid molecule encoding an EON as disclosed herein. Disclosed is also a pharmaceutical composition comprising an EON as disclosed herein, or a vector as disclosed herein, and a pharmaceutically acceptable carrier.

Disclosed herein is an EON, a vector, or a pharmaceutical composition as disclosed herein, for use in the treatment of a disorder, such as ASCVD, caused by elevated levels of LDL-C and/or triglycerides. Disclosed herein is a use of an EON, or a vector, as disclosed, in the manufacture of a medicament for the treatment, amelioration or prevention of ASCVD.

Disclosed is a method of treating, ameliorating, or preventing a disorder caused by elevated plasma levels of LDL-C and/or triglycerides, preferably ASCVD, in a patient in need thereof, the method comprising contacting an ANGPTL3 polynucleotide in a cell of the subject with an EON capable of effecting an ADAR-mediated adenosine to inosine alteration of an adenosine in a codon encoding an amino acid involved with heparin binding, more preferably the first adenosine in the codon for lysine (K) at position 63 in the mature ANGPTL3 protein, thereby altering the codon to a codon for glutamic acid (E) at position 63, thereby lowering or diminishing the ability of ANGPTL3 to inhibit lipolysis. Disclosed is also a method of treating, ameliorating, or preventing a disorder caused by elevated plasma levels of LDL-C and/or triglycerides, preferably ASCVD, the method comprising administering to a patient in need thereof a therapeutically effective amount of an EON as disclosed, a vector as disclosed, or a pharmaceutical composition as disclosed. Disclosed is also a method of editing an ANGPTL3 polynucleotide, the method comprising contacting the ANGPTL3 polynucleotide with an EON capable of effecting an adenosine deaminase acting on RNA (ADAR)-mediated adenosine to inosine alteration of an adenosine associated with heparin binding, thereby editing the ANGPTL3 polynucleotide. In one embodiment, the ANGPTL3 transcript that needs to be edited is from an ANGPTL3 wild-type gene. Disclosed is also a method of treating ASCVD caused by elevated plasma levels of LDL-C and/or caused by elevated levels of triglycerides, in a patient in need thereof, the method comprising contacting an ANGPTL3 polynucleotide in a cell of the subject with an EON capable of effecting an ADAR-mediated adenosine to inosine alteration of an adenosine in a codon associated with heparin binding, preferably the first adenosine in the AAG codon encoding lysine at position 63 of the ANGPTL3 protein, thereby treating the patient.

### BRIEF DESCRIPTION OF THE DRAWINGS

One or more embodiments of the disclosure will now be described, by way of example only, with reference to the accompanying drawings, in which:

FIG. 1 shows on top the human ANGPTL3 target RNA sequence (5′ to 3′; SEQ ID NO:31) with the lysine codon of position 63 underlined, and the target adenosine in this codon in bold face. Below the target sequence, the sequences (also 5′ to 3′) are given of the initial 30 EONs (EON #1 to #30; SEQ ID NO:1 to 30, respectively) that were designed for editing the target adenosine. All 30 EONs have a C:C mismatch at the +4 nucleotide position in the EON with the target sequence, and a G:G mismatch at the +5 nucleotide position in the EON. The target sequence where the EONs mismatch is given in grey. The chemical modifications in the EONs are as follows: t, a, g=2′-methoxyethoxy (2′-MOE) modified thymidine, adenosine, guanosine, respectively; c=2′-MOE modified 5-methyl-cytosine; mU, mA, mG, mC=2′-O-methyl(2′-OMe) modified uridine, adenosine, guanosine, cytosine, respectively; fU, fA, fG, fC=2′-fluoro (2′-F) modified uridine, adenosine, guanosine, cytosine, respectively; dA=deoxyadenosine; dZ=deoxycytidine analog carrying a Benner's base; “!”=PNdmi linkage; “*”=phosphorothioate linkage (PS); and (MeP)=methylphosphonate linkage. All 30 EONs also came in an identical version with the only difference that these comprise an RGN3 (tri-antennary) GalNAc moiety linked by a TEG linker to the 5′ terminus of the EON (EON #31 to 60, resembling EON #1 to 30, respectively).

FIG. 2 shows the percentage A to I editing of endogenous ANGPTL3 transcripts in human Huh-7 cells after gymnotic exposure to 30 EONs as indicated, in which RM5035 to RM5064 are EONs #1 to #30, respectively (see FIG. 1). A non-treated incubation and a PBS sample were taken along as negative controls.

FIG. 3 shows the percentage A to I editing of endogenous ANGPTL3 transcripts in human Huh-7 cells after gymnotic exposure to 30 EONs as in FIG. 2, with the addition of 1 μM AG1856 (a triterpene glycoside, or saponin; see WO2021/122998 and PCT/EP2024/051278, unpublished) to the culture medium. A non-treated incubation and an incubation with AG1856 were taken along as negative controls (AG1856 only and NT).

FIG. 4A shows the percentage A to I editing of endogenous ANGPTL3 transcripts in primary human hepatocytes (PHH's) that were grown into liver spheroids, after gymnotic exposure to the 30 EONs as in FIG. 2 and FIG. 3, with the addition of 1 μM AG1856 to the culture medium. Two negative controls were taken along (Mock and non-treated). FIG. 4B shows the results of a similar experiment in which spheroids were incubated with 5 μM EON, using only RM5059 (EON #25), RM5060 (EON #26), RM5061 (EON #27), RM5062 (EON #28), RM5063 (EON #29), and RM5064 (EON #30), but without the addition of the saponin.

FIG. 5 shows the percentage A to I editing of endogenous ANGPTL3 transcripts in PHH's that were incubated in the culture medium with 6 different EONs as indicated using three different concentrations in the medium (1, 5, and 10 μM). RM5035 (EON #1) is represented by the first three bars on the left, followed by RM5059 (EON #25), RM5060 (EON #26), RM5061 (EON #27), RM5062 (EON #28), and RM5063 (EON #29) towards the right in different grey tones.

FIG. 6 shows the percentage A to I editing of endogenous ANGPTL3 transcripts in liver spheroids, generated from PHH's, that were incubated for 7 days with 5 or 10 μM RM5059 without or with a GalNAc (GN) moiety attached to the 5′ terminus of the oligonucleotide. RM5059-GN is EON #55 (see the legend to FIG. 1).

FIG. 7 shows the normalized ANGPTL3 protein levels measured from western blot signals of human ANGPTL3 protein obtained from heparin-columns that received lysates from Huh-7 cells treated with RM5059, RM5035, and RM5047 oligonucleotides (EON #25, EON #1, and EON #13 in FIG. 1, respectively), in comparison to the mock-treated cells that were put at 100% in respect of heparin-bound ANGPTL3 protein.

FIG. 8A shows mouse Angptl3 target RNA sequence (5′ to 3′; SEQ ID NO:42) with the AAG codon corresponding to position 63 in the human amino acid sequence underlined, and the target adenosine in this codon in bold face. FIG. 8B shows the sequences (also 5′ to 3′) of five EONs with the respective RM names and SEQ ID NO's between brackets, that were used in an in vivo (mouse) study to determine editing and biomarker levels. All five EONs comprise a GalNAc moiety (L004; triantennary design as disclosed in WO2022/271806) attached to the 3′ terminus. The chemical modifications in these EONs are as follows: Um, Am, Gm, and Cm are 2′-OMe modified uridine, adenosine, guanosine, and cytidine, respectively; Ge is 2′-MOE modified guanosine; m5Ue is 2′-MOE modified 5-methyl-uridine (identical to a thymidine with a 2′-MOE substitution, or ‘Te’); Gf, Cf, Af, and Uf are 2′-F modified guanosine, cytidine, adenosine, and uridine, respectively; Ad is deoxyadenosine; Zd is a deoxynucleotide (deoxycytidine analog) carrying a Benner's base; “!” refers to PNdmi linkages; “\hat{ }” refers to a MP linkage; “0” refers to phosphodiester (PO) linkages; “*” refers to PS linkages.

FIG. 9A shows the editing percentages on day 7 (left bars) and at day 14 (right bars) in the liver of mice that were treated with PBS as a negative control and any of the EONs of FIG. 8B. FIG. 9B shows the editing percentages in the kidney of the same mice.

FIG. 10A shows the level of Angptl3 protein (μg/mL) on day 7 in the plasma of the same mice (see FIG. 9A), wherein PBS served as a negative control and the EONs were as indicated.

FIG. 10B shows the plasma LPL activity post-heparin on day 7 in nmol/mL/min in the same mice.

FIG. 10C shows the concentration of LDL-cholesterol (LDL-C) on day 7 in mmol/L in the same mice. FIG. 10D shows the amount of Apolipoprotein B (ApoB) on day 7 in mg/dl in the same mice.

FIG. 11 shows the RM numbers, SEQ ID NO's and sequences with modifications of 25 EONs that were designed to determine the influence of 2′-MOE substituted nucleotides at a variety of positions in relation to the RM5059 EON. The 2′-MOE variations are given in bold. On the 3′ side of the introduced 2′-MOE substituted nucleotide a PO linkage is introduced, except at the 5′ and 3′ termini of the EONs, where the PNdmi linkage was maintained. Also, the nucleotide positions +1 (already 2′-MOE modified), 0,-1,-2, and 3 were not amended. All modifications are as given in FIG. 8B.

FIG. 12A shows the editing percentages after 72 hrs gymnotic uptake of 1 μM EON in PHH's using the indicated EONs, in which RM5059 served as the positive control and standard (dotted line). A non-treated (NT) sample was taken as a negative control. The decrease with RM105748 in comparison to RM5059 is significant, as indicated. FIG. 12B shows the results of the same experiment but now using 5 μM EON, providing a similar tendency as seen with the 1 μM treated samples.

FIG. 13A shows again the mouse Angptl3 target sequence of SEQ ID NO:42 with the sequence and modifications of RM107387 (SEQ ID NO:45) and RM118133 (SEQ ID NO:1040) that only differ in the linkage position 0, where RM107387 comprises a PO linkage and RM118133 comprises a PS linkage. Also provided are three negative control EONs that should be less efficient in providing RNA editing: Control 1 (SEQ ID NO:1041) that comprises a deoxyguanosine (Gd) at the orphan position and wherein the remainder is identical to RM107387; Control 2 (SEQ ID NO: 1042) that comprises a Gd at the orphan position and wherein all Uf positions (present in RM107387) are replaced by Um; and Control 3 (SEQ ID NO: 1043) that comprises a cytidine with a 2′-OMe ribose substitution (Cm) at the orphan position, an adenosine with a 2′-OMe ribose substitution at nucleotide position −1, and a PS linkage (instead of a MP linkage) at linkage position −2. FIG. 13B shows the human ANGPTL3 target sequence (SEQ ID NO:31) and the sequences and modifications of RM5059 (again) and the 32 EONs that provided the highest level of RNA editing in a large high-throughput screen in PHH's. The SEQ ID NO's are given between brackets. The chemical modifications are as given in FIG. 8B. Mismatching positions are given with grey boxes. The target adenosines are underlined. All EON sequences are from 5′ to 3′.

### DETAILED DESCRIPTION

Angiopoietin-like 3 (ANGPTL3) is a hepatically secreted protein that acts as a potent inhibitor of LPL, the primary mechanism by which triglyceride-rich lipoproteins are cleared from the circulation. In addition, ANGPTL3 is an endogenous inhibitor of endothelial lipase (EL). Besides that, it has been shown that ANGPTL3 may also induce angiogenesis by binding to integrin αvβ₃ (Camenish G et al. J Biol Chem. 2002. 277:17281-17290). Loss of ANGPTL3 function appears to decrease triglyceride lipoprotein and HDL-C concentrations through loss of LPL and EL inhibition, respectively. The seemingly favourable implications of ANGPTL3 deficiency in reducing triglyceride concentrations and circulating LDL-C catalysed drug development programs aiming to inhibit ANGPTL3 with either monoclonal antibody (Evinacumab) and an antisense oligonucleotide (Vupanorsen).

The inventors of the present invention realized that another approach is possible to lower the activity (or preferably completely block the activity) by targeting the ANGPTL3 transcript such that its inhibitory activity on lipases is lowered, but the protein's other functions related to angiogenesis are maintained.

Disclosed herein is an approach that, in contrast to the monoclonal antibody and siRNA (or gapmer) approach, targets the transcript and predominantly influences ANGPTL3's lipase inhibitory activity, but allowing expression of a mutated protein that can still execute its activities unrelated to lipase inhibition. Several loss-of-function mutants, or mutants with lowered lipase inhibitory activity have been described, such as Gly253Cys, Leu127Phe, Ile333Ser, Asp290His, Cys408Arg, Ser292Pro, Asp70Asn, Tyr250Cys, Lys63Thr, Asp42Asn, and Thr383Ser (Stitziel N O et al. J Am Coll Cardiol. 2017. 69 (16): 2054-2063). The Lys63Thr mutation is of particular interest and subject to the present disclosure.

Disclosed herein is an antisense oligonucleotide (generally abbreviated to EON) conducive to ADAR-mediated adenosine deamination and capable of forming a double-stranded complex with a region of an endogenous human ANGPTL3 transcript molecule (such as pre-mRNA or mRNA) in a human liver cell (such as an hepatocyte), wherein the region of the ANGPTL3 transcript molecule comprises a target adenosine, and wherein the double-stranded complex can recruit an endogenous ADAR enzyme (such as ADAR1 or ADAR2) to deaminate the target adenosine (A) into an inosine (I), thereby editing the ANGPTL3 transcript molecule, wherein the target A is present in a codon encoding an amino acid involved in the lipase inhibition functionality of the ANGPTL3 protein, wherein the deamination of the target A into an I results in an ANGPTL3 protein that is impaired in its lipase inhibitory function, and wherein the target A is the first A in the codon (AGA) encoding arginine at position 221 in the human ANGPTL3 protein. This generates an IGA codon, which is read by the translation machinery of the cell as GGA, which codes for glycine. Hence, this RNA editing results in a R221G mutation in the human ANGPTL3 protein. In one embodiment, in a double editing event (and using an EON that causes a double RNA editing), the AGA codon for position 221 can also be edited to IGI (read as GGG), which also codes for glycine. The arginine at position 221 is in a furin cleavage site and changing this arginine to a glycine will abrogate cleavage and thereby lowers ANGPTL3 activity, and ultimately will result in lower plasma LDL-C and/or triglyceride levels.

Disclosed herein is an EON conducive to ADAR-mediated adenosine deamination and capable of forming a double-stranded complex with a region of an endogenous human ANGPTL3 transcript molecule (such as pre-mRNA or mRNA) in a human liver cell (such as an hepatocyte), wherein the region of the ANGPTL3 transcript molecule comprises a target adenosine, and wherein the double-stranded complex can recruit an endogenous ADAR enzyme (such as ADAR1 or ADAR2) to deaminate the target A into an I, thereby editing the ANGPTL3 transcript molecule, wherein the target A is present in a codon encoding an amino acid involved in the lipase inhibition functionality of the ANGPTL3 protein, wherein the deamination of the target A into an I results in an ANGPTL3 protein that is impaired in its lipase inhibitory function, and wherein the target A is the first A in the codon (AGA) encoding arginine at position 224 in the human ANGPTL3 protein. This generates an IGA codon, which is read by the translation machinery of the cell as GGA, which codes for glycine. Hence, this RNA editing results in a R224G mutation in the human ANGPTL3 protein. In one embodiment, in a double editing event (and using an EON that causes a double RNA editing), the AGA codon for position 224 can also be edited to IGI (read as GGG), which also codes for glycine. The arginine at position 224 is in a furin cleavage site and changing this arginine to a glycine will abrogate cleavage and thereby lowers ANGPTL3 activity, and ultimately will result in lower plasma LDL-C and/or triglyceride levels.

In yet another embodiment, the codons for arginine positions 221 and 224 can be edited by a single EON having the structure, chemical modifications, and orphan nucleotides opposite the target adenosines according to the teaching provided herein. Changing both arginine residues at the same time will lower the furin cleavage possibility even further, influencing the ANGPTL3 functionality in a negative manner even more. Although not required, double editing of both arginine codons is potentially possible. This is not needed because if the editing of the first adenosine (for each codon) occurs, glycine residues are already encoded.

The Lys63Thr mutation (discussed above) has been found in only a very small number of individuals. Importantly, Stitziel et al. (2017) showed a dramatic decrease in activity of the protein when introduced in mice. Others showed that position 63 is present in a putative six amino acid containing heparin binding site and that mutating this putative heparin binding site, in which the lysine on position 63 was mutated to an asparagine caused a significant decrease in plasma triglyceride concentration in mice (Ono M et al. J Biol Chem. 2003. 278 (43): 41804-41809). Although it has not been shown that these mutations cause a potential loss in heparin binding (it may be that another factor involved in lipolysis interacts with this part of the protein), the effect on the ANGPTL3 inhibitory function was significant.

The inventors of the present invention reasoned that changing the positively charged lysine (Lys; K) at position 63 to a negatively charged amino acid, such as glutamic acid (Glu; E), may further lower the inhibitory activity of the protein. The inventors reasoned that using an antisense oligonucleotide that recruits an endogenous deamination moiety, for the specific deamination of the first A in the codon for lysine at position 63 (AAG) and editing this A to an I, which is a nucleotide that is seen as a guanosine (G) by the translation machinery (hence AAG >IAG >GAG) should result in an ANGPTL3 protein with a lowered (diminished or potentially absent) lipase inhibitor activity. This technology is generally referred to as ‘RNA editing’. Disclosed herein are oligonucleotides that can be used to specifically deaminate a specific target A in the transcript of the (human) wild-type ANGPTL3 transcript (pre-mRNA and/or mRNA) in vivo, preferably using endogenous deaminating enzymes, to produce an ANGPTL3 protein that is negatively influenced in its ability to inhibit LPL (and potentially EL), therethrough allowing the breakdown of LDL-C and triglycerides, and thereby lowering the risk of cardiovascular disease. The editing of the first A in the codon for lysine at position 63 is used as a preferred embodiment. Other positions within the ANGPTL3 protein may also be involved in inhibiting LPL and/or EL, and such positions may be altered in a similar manner as described herein. It is preferred that the deamination of a particular A in the ANGPTL3 transcript predominantly affects the inhibitory effect of the ANGPTL3 protein and does not cause a complete loss of function, especially in the angiogenesis functionality of ANGPTL3. However, based on the current teaching and based on what is known in the field about ANGPTL3 and its specific functions and structure, the skilled person will be able to determine whether a certain RNA editing of a specific A in the transcript will yield a similar effect. Hence, the deamination of the first A in the lysine 63 codon serves as a preferred example herein.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are A to I conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. As mentioned above, I is read as G by the translational machinery of the cell, meaning that, if an edited A is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing A deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. Proc Natl Acad Sci USA. 1995, 92:8298-8302; Montiel-Gonzalez et al. Proc Natl Acad Sci USA. 2013, 110 (45): 18285-18290; Vogel et al. Angewandte Chemie 2014, Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all A's in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl(2′-OMe) modified nucleosides in the oligonucleotide at positions opposite to A's that should not be edited and used a non-modified nucleoside directly opposite to the specifically targeted A on the target RNA. However, the specific editing effect at the target nucleotide has not been shown to take place without the use of recombinant ADAR enzymes having covalent bonds with the EON. Several publications have now shown that the recruitment of endogenous ADAR (hence without the need for an exogenous and/or recombinant source) is feasible while maintaining a specificity in which a single A within a target RNA molecule can be targeted and deaminated to an I. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target A, but in other embodiments AONs (or “RNA editing oligonucleotides”, often abbreviated to ‘EONs’) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The ‘orphan nucleoside’, which is defined as being the nucleotide in the EON that is positioned directly opposite the target A in the target RNA molecule, did not carry a 2′-OMe modification. The orphan nucleotide can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific A's within such RNA target molecules, be it to repair a mutation that resulted in a premature stop codon, or other mutation causing disease. Examples of such disclosures in which A's are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); WO2021/243023 (SERPINA1); and WO2023/152371 (PCSK9).

Disclosed herein are EONs that can produce RNA editing of a target A in a wild-type (and if needed also in a mutant) ANGPTL3 transcript molecule (pre-mRNA and/or mRNA), through which the resulting ANGPTL3 protein is affected negatively in its ability to inhibit lipases, which in turn results in the decrease in plasma LDL-C and triglyceride concentrations. In a preferred aspect, the EON causes the deamination of the A present at the first position of the codon encoding lysine at position 63 of the mature human protein, thereby generating an I. In other words, the AAG codon encoding lysine (K or Lys; wildtype form) at amino acid position 63 is converted to an IAG codon, which is read as GAG that encodes glutamic acid (E or Glu; mutant form). The change is often referenced to as K63E, or Lys63Glu, which as far as the inventors are aware, is a change that has not been found in nature thus far. However, as mentioned above, a Lys63Thr mutation in human ANGPTL3 has been found (Stitziel et al. 2017).

In another embodiment, an EON according to the present disclosure causes the deamination of another A present in the ANGPTL3 transcript, which may be any A that, when deaminated into an I, results in an ANGPTL3 protein with a loss-of-function in respect of its lipase-inhibitory activity. Herein, the transcript numbering of the Homo sapiens ANGPTL3 protein and gene found in NCBI is used (transcript NM_014495), which means that the change after RNA editing is referred to as K63E in the protein and could potentially be referred to as c. 187A>G in the transcript.

Although in a preferred embodiment the EON of the present disclosure is a single-stranded oligonucleotide comprising an orphan nucleotide opposite the target adenosine, wherein the orphan nucleotide is chemically modified as disclosed herein, and wherein the remainder of the oligonucleotide is chemically modified to prevent it from nuclease breakdown also as disclosed herein, in another embodiment, the disclosure relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is expressed through a vector, such as an adeno-associated virus (AAV), or wherein the oligonucleotide is in a circular format. It is to be understood that any kind of oligonucleotide-based RNA editing is encompassed by the present disclosure if it relates to the deamination of a nucleotide in the ANGPTL3 transcript, preferably the change causing K63E, and causes the loss of inhibitory function of the ANGPTL3 protein. In a preferred aspect, the EON of the present disclosure is a ‘naked’ oligonucleotide, comprising a variety of chemical modifications in the ribose sugar, the base, and/or the internucleoside linkage of one or more of the nucleotides within the sequence, that can hybridize to the ANGPTL3 transcript or a part thereof that includes the target A, and can recruit endogenous ADAR for the deamination of the target A.

The disclosure relates to an EON conducive to ADAR-mediated A deamination and capable of forming a double-stranded complex with a region of an endogenous human ANGPTL3 transcript molecule in a cell, wherein the region of the ANGPTL3 transcript molecule comprises a target A, and wherein the double-stranded complex can recruit an endogenous ADAR enzyme to deaminate the target A into an I, thereby editing the ANGPTL3 transcript molecule. The endogenous ADAR enzyme is preferably ADAR1 or ADAR2. The cell is preferably a human liver cell, more preferably a human hepatocyte. The ANGPTL3 transcript molecule is preferably a pre-mRNA or an mRNA molecule. The EON of the present disclosure preferably targets an A for deamination to cause a dysfunction of the resulting ANGPTL3 protein, preferably in its ability to inhibit lipolysis. Although several mutations have been identified that cause a dysfunction of the ANGPTL3 protein, a preferred position that is targeted through the EONs as disclosed herein is the A at the first position of the codon for lysine at position 63 in the mature protein, leading to a mutant K63E ANGPTL3 protein (and thereby a c. 187A>G mutation in the transcript). The EONs as disclosed herein are capable of deaminating the first A in the AAG codon encoding lysine at position 63 in the mature human ANGPTL3 protein, thereby generating an IAG codon, which is translated to glutamic acid (E) because the codon is read as GAG. In one embodiment, the EON as disclosed herein comprises or consists of the sequence of any one of the EONs disclosed herein that target a human ANGPTL3 transcript, or a part thereof that includes position 187. In one embodiment, the EON is chemically modified as disclosed herein. In one embodiment, the EON is (non) covalently bound to a GalNAc moiety, especially when targeting hepatocytes is desired. The GalNAc moiety may be attached to the 5′ terminus and/or the 3′ terminus of the EON. Attached moieties include the use of hydrophobic moieties (such as tocopherol and cholesterol) together with cell-specific ligands (such as GalNAc moieties), that have also been described herein, and in detail in PCT/EP2023/079290 (unpublished), which may either be bound to the EON or its opposite strand, or both. Preferred GalNAc moieties that can be used in the context of the EONs as disclosed herein are disclosed in WO2022/271806. In one embodiment, a GalNAc moiety is bound to the EON as disclosed herein via a TEG linker or through a PO bond.

In one embodiment, an EON according to the disclosure comprises at least one nucleotide comprising one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. In an embodiment, the one or more additional modifications in the linkage moiety is each independently selected from a PS, phosphonoacetate, phosphorodithioate, methylphosphonate (MP; or MeP), sulfonylphosphoramidate, PNms, or a (1,3-dimethylimidazolidin-2-ylidene) phosphoramidate (PNdmi) internucleoside linkage. Preferably, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl;-methoxy;-aminopropoxy;-methoxyethoxy;-dimethylamino oxyethoxy; and -dimethylaminoethoxyethoxy. In an embodiment, the EON comprises one or more mismatches, wobbles, or bulges, wherein a single mismatch may be present when the target adenosine has (for example) an opposite cytidine in the EON. If the orphan nucleotide is a cytidine, that cytidine does not comprise a 2′-OMe ribose substitution. In an embodiment, the EON comprises a C:C mismatch at nucleotide position +4 in the EON, and/or a G:G mismatch at nucleotide+5 in the EON, wherein the mismatch is with the human target ANGPTL3 transcript molecule.

Disclosed is an EON conducive to ADAR-mediated adenosine deamination and capable of forming a double-stranded complex with a region of an endogenous human ANGPTL3 (pre-) mRNA molecule in a cell, wherein the region of the (pre-) mRNA molecule comprises a target A that is the first nucleotide in the codon encoding lysine at position 63 in the human mature ANGPTL3 protein and wherein the double-stranded complex can recruit an endogenous ADAR enzyme to deaminate the target A into an I, thereby editing the ANGPTL3 (pre-) mRNA molecule, wherein the cell is a human hepatocyte, wherein the deamination of the target A into an I results in an ANGPTL3 protein that is impaired in its lipase inhibitory function, and wherein the EON is preferably selected from the group consisting of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, and 29.

In a preferred embodiment, an EON of the present disclosure, as outlined intra, has an a-symmetrical design in which there are more nucleotides at the 5′ side of the orphan nucleotide than there are at the 3′ side of the orphan nucleotide in the EON (opposite the area that is targeted in the human ANGPTL3 transcript molecule). In a preferred embodiment, the EON as disclosed herein is 29, 30, 31, or 32 nucleotides in length, wherein the orphan nucleotide is nucleotide position 0 and there are preferably 5, 6, or 7 nucleotides at the 3′ side of the orphan nucleotide and preferably 22, 23, or 24 nucleotides at the 5′ side of the orphan nucleotide.

In a preferred embodiment, an EON of the present disclosure has the structure (X):

(X):
5′-N₂₄N₂₃N₂₂N₂₁N₂₀N₁₉N₁₈N₁₇N₁₆N₁₅N₁₄N₁₃N₁₂N₁₁N₁₀N₉N₈N₇N₆N₅N₄N₃
N₂N₁^{θ}ZdAd^{∧}M₂M₃M₄M₅M₆M₇-3′

wherein:
   Zd is the orphan nucleotide at nucleotide position 0, which is a deoxynucleotide carrying a Benner's base;
   N₁ is a thymidine (5-methyluridine with a deoxyribose; m5Ud; or Td), or a 5-methyluridine with a 2′-MOE ribose substitution (m5Ue; or Te);
   N₂ is a cytidine with a 2′-fluoro (Cf) or a 2′-OMe (Cm) ribose substitution;
   N₃ is a uridine with a 2′-fluoro (Uf) or a 2′-OMe (Um) ribose substitution;
   N₄ is a cytidine with a 2′-fluoro ribose substitution (Cf), or a guanosine with a 2′-fluoro (Gf) or a 2′-OMe (Gm) ribose substitution;
   N₅ is a guanosine with a 2′-fluoro ribose substitution (Gf), or a cytidine with a 2′-fluoro (Cf) or a 2′-OMe (Cm) ribose substitution;
   N₆, N₇, N₁₃, N₁₄, N₁₅, N₁₈, N₁₉, N₂₂, and M₂ are uridine with either a 2′-fluoro ribose substitution (Uf) or a 2′-OMe ribose substitution (Um);
   N₈, N₉, N₁₀, and N₂₁ are cytidine with either a 2′-fluoro ribose substitution (Cf) or a 2′-OMe ribose substitution (Cm);
   N₁₁, N₁₂, and M₃ are guanosine with either a 2′-fluoro ribose substitution (Gf) or a 2′-OMe ribose substitution (Gm);
   M₄ is guanosine with a 2′-fluoro ribose substitution (Gf), a 2′-OMe ribose substitution (Gm), or a 2′-MOE ribose substitution (Ge);
   N₁₆, N₁₇, N₂₀, and M₅ are adenosine with either a 2′-fluoro ribose substitution (Af) or a 2′-OMe ribose substitution (Am);
   N₂₃ and N₂₄ are absent; or N₂₃ is Gf or Gm, and N₂₄ is absent; or N₂₃ is Gf or Gm, and N₂₄ is Um;
   M₆ and M₇ are absent; or M₆ is Cm and M₇ is absent; or M₆ is Cm and M₇ is Am or Af;
   Ad is deoxyadenosine;
   θ is at linkage position 0, and is a PO linkage;
   \hat{ } is at linkage position −2 and is an MP or a PNms linkage;
   linkage positions-3 and 5 are, each independently, a PO or a PS linkage, preferably a PO linkage;
   linkage position +23 is a PNdmi or a PNms linkage;
   linkage position −7 is a PNdmi or a PNms linkage; and
   all other linkages are PS linkages.

In a preferred embodiment, the EON of the present disclosure, such as for example those of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, 29, and those of structure (X) above, are bound to a GalNAc moiety for improved delivery to hepatocytes, more preferably to a tri-antennary GalNAc moiety as disclosed herein, that is preferably attached to the 3′ end of the EON, either directly or through a linker, such as a TEG linker.

In a preferred embodiment, the EON of the present disclosure, such as for example those of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, 29, and those of structure (X) above, are ‘packaged’ into a delivery vehicle, preferably a lipid nanoparticle (LNP), for improved in vivo delivery.

In a preferred embodiment, the EON of the present disclosure, such as for example those of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, 29, and those of structure (X) above, are conjugated to AG1856, for improved endosomal release, and/or intracellular trafficking, after cell entry.

Disclosed herein is a vector, preferably a viral vector, more preferably an adeno-associated virus (AAV) vector, comprising a nucleic acid molecule encoding an EON as disclosed herein. Further disclosed herein is a pharmaceutical composition comprising an EON as disclosed, or a vector as disclosed, and a pharmaceutically acceptable carrier.

In an embodiment, disclosed is an EON as disclosed herein, a vector as disclosed herein, or a pharmaceutical composition as disclosed herein, for use in the treatment, amelioration, or prevention of a disorder caused by elevated plasma levels of LDL-C and/or triglycerides, such as ASCVD.

In an embodiment, disclosed is a method of treating, ameliorating, or preventing a disorder caused by elevated plasma levels of LDL-C and/or triglycerides, such as ASCVD, in a patient in need thereof, the method comprising contacting an ANGPTL3 polynucleotide in a cell of the subject with an EON capable of effecting an ADAR-mediated A to I alteration of an A in a codon encoding an amino acid involved with heparin binding, more preferably the first A in the codon for lysine at position 63 in the mature ANGPTL3 protein, thereby altering the codon to a codon for glutamic acid at position 63 (K63E), thereby lowering or diminishing the ability of ANGPTL3 to inhibit lipolysis. Disclosed is also a method of treating, ameliorating, or preventing a disorder caused by elevated plasma levels of LDL-C and/or triglycerides, preferably ASCVD, the method comprising administering to a patient in need thereof a therapeutically effective amount of an EON as disclosed, a vector as disclosed, or a pharmaceutical composition as disclosed. Disclosed is also a method of editing an ANGPTL3 polynucleotide, the method comprising contacting the ANGPTL3 polynucleotide with an EON capable of effecting an ADAR-mediated A to I alteration of an A associated with heparin binding, thereby editing the ANGPTL3 polynucleotide. In one embodiment, the ANGPTL3 transcript that needs to be edited is from an ANGPTL3 wild-type gene. Disclosed is also a method of treating ASCVD caused by elevated plasma levels of LDL-C and/or caused by elevated levels of triglycerides, in a patient in need thereof, the method comprising contacting an ANGPTL3 polynucleotide in a cell of the subject with an EON capable of effecting an ADAR-mediated A to I alteration of an A in a codon associated with heparin binding, preferably the first A in the AAG codon encoding lysine at position 63 of the ANGPTL3 protein, thereby treating the patient.

In an embodiment, disclosed is a method for the deamination of a target A in an ANGPTL3 pre-mRNA or mRNA molecule in a cell, the method comprising the steps of: (i) providing the cell with an EON or a vector as disclosed herein; (ii) allowing uptake by the cell of the EON or the vector, respectively; (iii) allowing annealing of the EON to the ANGPTL3 pre-mRNA or mRNA molecule; (iv) allowing an endogenous ADAR enzyme, such as ADAR1 or ADAR2, to deaminate the target A in the target RNA molecule to an I; and optionally (v) identifying the presence of the I in the target RNA molecule. A preferred target A is the first nucleotide in the transcript molecule coding for lysine at position 63 in the mature protein. Preferably, step (v) comprises a) determining the sequence of the ANGPTL3 pre-mRNA or mRNA molecule; b) assessing the presence of a mutant ANGPTL3 protein; or c) using a biomarker read-out, such as assessing LDL activity, or assessing a level of LDL-C and/or triglycerides in the plasma of the subject that is treated, or any other biomarker related to the lipase-inhibitory function of ANGPTL3 known to the person skilled in the art. Examples of biomarker assessment are given in the accompanying in vivo experiments disclosed below.

### Definitions

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a PO, phosphonoacetate, phosphotriester, PS, phosphoro (di) thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m5U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an EON as disclosed herein may comprise a mix of ribonucleosides and deoxyribonucleosides. When a deoxyribonucleotide is used, hence without a modification at the 2′ position of the sugar (the ribose), the nucleotide is often abbreviated to dA (Ad), dC (Cd), dG (Gd) or T (m5Ud) in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2, N2-dimethylguanosine, N2, N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be PO linkages as well as modifications thereof, including, phosphonoacetate, phosphotriester, PS, phosphoro (di) thioate, MP, phosphoramidate linkers, phosphoryl guanidine, thiophosphoryl guanidine, sulfono phosphoramidate, PNms, PNdmi, and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x+10%.

The term ‘conducive to’ can be used interchangeably with ‘capable of facilitating’.

The word ‘substantially’ does not exclude ‘completely’, e.g., a composition which is ‘substantially free from Y’ may be completely free from Y. Where relevant, the word ‘substantially’ may be omitted from the definition of the disclosure.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex, or HEON, with another complementary nucleic acid strand), or when it forms a double stranded complex with the target (pre-) mRNA molecule. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target A. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target. Disclosed herein are multiple EONs that target an A in the human ANGPTL3 transcript molecule, while having two mismatches (at position +4 and/or +5) besides the interaction between the orphan nucleotide and the target A.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments first nucleic acid strands of the present disclosure comprise fewer than four mismatches with the target sequence, for example 0, 1 or 2 mismatches. ‘Wobble’ base pairs are G-U, I-U, I-A, and I-C base pairs. Although a G:G pairing would be considered a mismatch, that does not necessarily mean that the interaction is unstable, which means that the term ‘mismatch’ may be somewhat outdated based on the current disclosure where a Hoogsteen base-pairing may be seen as a mismatch based on the origin of the nucleotide but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An EON (and the complementary nucleic acid strand when two oligonucleotides form a HEON) as disclosed herein may be chemically modified almost in its entirety, for example by providing nucleotides with a ribose sugar moiety carrying a 2′-OMe substitution, a 2′-F substitution, or a 2′-MOE substitution. The orphan nucleotide in the EON is preferably a cytidine or analog thereof (such as a nucleotide carrying a Benner's base), or a uridine or analog thereof (such as iso-uridine), and/or in one embodiment comprises a diF modification at the 2′ position of the sugar, in another embodiment comprises a deoxyribose (2′-H, DNA), and in yet a further embodiment, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used in accordance with the disclosure. The regular internucleoside linkages between the nucleotides may be altered by mono- or di-thioation of the PO bonds to yield PS esters or phosphorodithioate esters, respectively. Other modifications of the internucleoside linkages are possible, including amidation and peptide linkers.

In an embodiment, the EON of the present disclosure comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. Notably, when the EON is delivered through a (viral) vector, the length may increase as being longer than 60 nucleotides. However, when the EON is to be delivered as is, without a vector, also referred to as a ‘naked form’, the length of the EON is limited to 15 to 60 nucleotides to reduce the risk of degradation. Furthermore, in a naked form, the EON is preferably chemically modified as outlined herein to lower the risk of degradation.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate A's in a non-discriminative manner, reacting with any A it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

The RNA editing proteins present in the cell that are of most interest to be used with an EON of the present disclosure are human ADAR1 and ADAR2. It will be understood by a person having ordinary skill in the art that the extent to which the editing entities inside the cell are redirected to other target sites may be regulated by varying the affinity of the first nucleic acid strand for the recognition domain of the editing molecule. The exact modification may be determined through some trial and error and/or through computational methods based on structural interactions between the EON and the recognition domain of the editing molecule. In addition, or alternatively, the degree of recruiting and redirecting the editing entity resident in the cell may be regulated by the dosing and the dosing regimen of the EON. This is something to be determined by the experimenter (in vitro) or the clinician, usually in phase I and/or II clinical trials.

The disclosure concerns the modification of target RNA sequences in eukaryotic, preferably metazoan, more preferably mammalian, even more preferably human cells, and most preferably human liver cells such as hepatocytes. The disclosure is particularly suitable for modifying RNA sequences in cells and tissues in which ANGPTL3 is expressed and from which it is secreted. According to the art, these are mainly hepatocytes. Because ANGPTL3 is predominantly produced and is secreted from liver cells for lipase inhibition, the preferred target cell for the EONs of the present disclosure are liver cells, more preferably hepatocytes. The target cell can be located in vitro, ex vivo or in vivo. One advantage of the disclosure is that it can be used with cells in situ in a living organism, but it can also be used with cells in culture. In some embodiments cells are treated ex vivo and are then introduced into a living organism (e.g., re-introduced into an organism from whom they were originally derived). The disclosure can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through human ADAR2 for example is thought to take place on primary transcripts in the nucleus, during transcription or splicing, or in the cytoplasm, where e.g., mature mRNA, miRNA or ncRNA can be edited.

It should be clear, that targeted editing according to the disclosure can be applied to any A within the ANGPTL3 transcript if the deamination of the A results in a decrease of ANGPTL3 lipase inhibition functionality. As outlined herein, it is preferred to target the first A that is present in a codon for lysine at position 63 of the mature protein.

Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present disclosure. Hence, any RNA editing of a target A in the ANGPTL3 transcript and that results in decrease or absence of the ANGPTL3 protein function in lipase inhibition is encompassed by the present disclosure. The present disclosure opens a whole new field of treating ASCVD, especially those caused by too high levels of LDL-C and/or triglycerides, using RNA editing techniques.

The amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker (such as plasma levels of LDL-C for example). It is possible that higher doses of EONs could compete for binding to an ADAR within a cell, thereby depleting the amount of the entity, which is free to take part in RNA editing, but routine dosing trials will reveal any such effects for a given EON and a given target. The addition of cell-targeting moieties such as GalNAc conjugates that are suitable for targeting hepatocytes may contribute to the determination of a suitable amount that can be administered for a therapeutic effect. Similarly, the use of other vehicles, such as LNP's (see further below) that may be used to ‘package’ the EONs of the present disclosure may also contribute to the determination of how much EON provides a desired in vivo effect.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. As mentioned above, plasma level concentrations of LDL-C and/or triglycerides in a sample from a treated subject is a proper biomarker for assessing the function of the ANGPTL3 protein in the subject, before and after treatment, or with or without treating the subject with an EON or vector as disclosed herein. After this trial has been performed once then the knowledge can be retained, and future delivery can be performed without needing to take biopsy samples. A method of the disclosure can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, as indicated above, the change may be assessed on the function of the protein, for instance by measuring or assessing a plasma LDL-C concentration before, during, and/or after treatment or assessing any other potential marker, which measurements are preferably performed in vitro on samples obtained from the treated subject. Another suitable biomarker that is linked to the amount of LDL-C is the activity of LPL that can be readily determined in blood samples from the treated subject.

After RNA editing has occurred in a cell, the modified RNA can become diluted over time, for example due to cell division, limited half-life of the edited RNAs, etc. Thus, in practical therapeutic terms a method of the disclosure may involve repeated delivery of an EON until enough target RNAs have been modified to provide a tangible benefit to the patient and/or to maintain the benefits over time.

EONs of the disclosure are particularly suitable for therapeutic use, and so the disclosure also relates to a pharmaceutical composition comprising an EON of the disclosure, or a vector or plasmid encoding the EON of the disclosure, and a pharmaceutically acceptable carrier. In some embodiments of the disclosure the pharmaceutically acceptable carrier can simply be a saline solution. This can usefully be isotonic or hypotonic, particularly for pulmonary delivery. The disclosure also provides a delivery device (e.g., syringe, inhaler, nebuliser) which includes a pharmaceutical composition of the disclosure.

The disclosure also provides an EON of the disclosure for use in a method for introducing a mutation in a target ANGPTL3 RNA sequence in a mammalian, preferably a human liver cell, as described herein. Similarly, the disclosure provides the use of an EON of the disclosure in the manufacture of a medicament for making a change in a target ANGPTL3 RNA sequence in a mammalian, preferably a human liver cell, as described herein, and thereby treating, preventing, or ameliorating diseases related to risky levels of LDL-C and/or triglycerides.

The disclosure also relates to a method for the deamination of at least one specific target A present in a target ANGPTL3 RNA sequence in a cell, the method comprising the steps of: providing the cell with an EON according to the disclosure; allowing uptake by the cell of the EON; allowing annealing of the EON to the target RNA molecule; allowing a mammalian ADAR enzyme comprising a natural dsRNA binding domain as found in the wild type enzyme to deaminate the target A (preferably the first A of the codon encoding lysine at position 63 in the mature protein) in the target RNA molecule to an I; and optionally identifying the presence of the I in the RNA sequence.

The disclosure also relates to a method for the deamination of at least one specific target A present in a target ANGPTL3 RNA sequence in a cell, the method comprising the steps of: providing the cell with a vector or plasmid encoding the EON according to the disclosure; allowing uptake by the cell of the vector or plasmid; allowing annealing of the EON to the target RNA molecule; allowing a mammalian ADAR enzyme comprising a natural dsRNA binding domain as found in the wild type enzyme to deaminate the target A (preferably the first A of the codon encoding lysine at position 63 in the mature protein) in the target RNA molecule to an I; and optionally identifying the presence of the I in the RNA sequence.

In a preferred aspect, depending on the ultimate deamination effect of A to I conversion, the identification step comprises the following steps: sequencing the target RNA; assessing the presence or absence of a non-, or less-functional protein; assessing whether splicing of the pre-mRNA was altered by the deamination; or using a functional read-out, because the target RNA after the deamination should encode a protein with a lower or absent lipase inhibitory functionality.

Examples are assessing LDL-C and/or triglyceride concentrations in (blood) samples after RNA editing. The identification of the deamination into I may therefore be a functional read-out using a suitable biomarker. The functional assessment mentioned herein will generally be according to methods known to the skilled person, while it may also be feasible to assess plaque formation which is generally caused by high levels of LDL-C and triglycerides over time. A suitable manner to identify the presence of an I after deamination of the target A is of course dPCR or even sequencing, using methods that are well-known to the person skilled in the art. However, the person skilled in the art of liver disease will preferably apply tests to monitor certain biomarkers related to lipase inhibition, as discussed above.

The EON according to the disclosure is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, a method according to the disclosure comprises the steps of administering to the subject an EON or pharmaceutical composition according to the disclosure, allowing the formation of a double stranded nucleic acid complex of the EON with its specific complementary target nucleic acid molecule in a cell in the subject; allowing the engagement of an endogenously present (=naturally present) adenosine deaminating enzyme, such as ADAR1 or ADAR2; and allowing the enzyme to deaminate the target A in the target nucleic target molecule to an I, thereby alleviating, preventing or ameliorating the disease related to high or increased LDL-C and/or triglyceride levels. The diseases that may be treated according to this method are preferably, but not limited to, the diseases listed herein, and any other disease in which deamination of an adenosine in ANGPTL3 transcripts would diminish or lower the protein's function in lipase inhibition.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the disclosure may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 100 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs according to the disclosure are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed herein that may be used in the EON of the present disclosure may also be used for a sense strand that is complementary to the EON, when the EON and the complementary strand form a so-called heteroduplex RNA editing oligonucleotide (HEON) complex, as described in PCT/EP2023/079290 (unpublished), except that the opposite sense strand does not have an orphan nucleotide. Hence, the modification related to the orphan nucleotide relate only to the EON of the present disclosure, but all other modifications relate to the EON of the present disclosure and any (protecting) sense oligonucleotide that may be used together with the EON in a pharmaceutical product. This includes the use of hydrophobic moieties (such as tocopherol and cholesterol) and cell-specific ligands (such as GalNAc moieties), that have also been described herein, and in detail in PCT/EP2023/079290 (unpublished), which may either be bound to the EON or its opposite strand, or both. Particularly preferred GalNAc moieties that can be used in the context of the EONs of the present disclosure are disclosed in WO2022/271806.

The internucleoside linkages in the oligonucleotides of the present disclosure may comprise one or more naturally occurring internucleoside linkages and/or modified internucleoside linkages. Without limitations, at least one, at least two, or at least three internucleoside linkages from a 5′ and/or 3′ end of the EON are preferably modified internucleoside linkages. A preferred modified internucleoside linkage is a PS linkage. In one embodiment, all internucleoside linkages of the EON are modified internucleoside linkages. In one embodiment, the EON comprises a PNdmi linkage linking the most terminal nucleoside at the 5′ and/or 3′ end, and the one before last nucleoside at each of these ends, respectively. The common chemical name for PNdmi is (1,3-dimethylimidazolidin-2-ylidene) phosphoramidate. A PNdmi linkage as preferably used in the EONs of the present disclosure has the structure of the following formula (I):

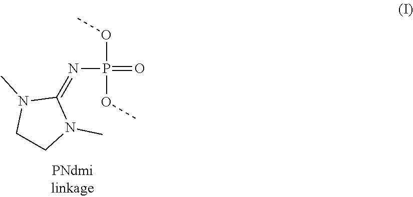

In one aspect, the EON as disclosed herein comprises at least one MP internucleoside linkage according to the following formula (II):

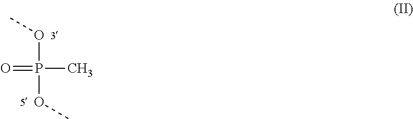

A preferred position for an MP linkage in an EON as disclosed herein is linkage position-2, thereby connecting the nucleoside at position −1 with the nucleoside at position −2, although other positions for MP linkages are not explicitly excluded.

An EON as disclosed herein may also comprise one or more linkage modifications according to the structure of the following formula (III):

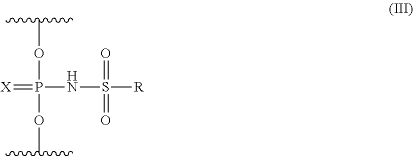

wherein:
X═O or S; and
R=an aryl, a substituted aryl, a heterocycle, a substituted heterocycle, an aromatic heterocycle, a substituted aromatic heterocycle, a C₁-C₆ alkoxy, a substituted C₁-C₆ alkoxy, a C₁-C₂₀ alkyl, a substituted C₁-C₂₀ alkyl, a C₁-C₆ alkenyl, a C₁-C₆ substituted alkenyl, a C₁-C₆ alkynyl, a substituted C₁-C₆ alkynyl, or a conjugate group. In a preferred embodiment, X═O and R=methyl and the linkage modification is referred to as “mesyl phosphoramidate”, “MsPA” or “PNms”. In one embodiment, a PNms linkage is used instead of the MP and/or PNdmi linkages.

In an embodiment, the EON as disclosed herein comprises an internucleoside linkage of the structure of formula (III), wherein X═O and R═CH₃, which linkage is generally referred to herein as a PNms linkage (mesyl phosphoramidate). In other preferred aspects, R equals one of the following structures (a), (b), (c), (d), (e), (f), (g), (h), or (i):

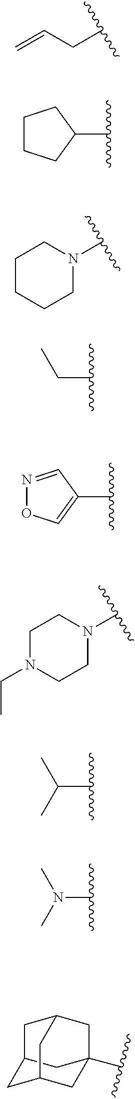

Other internucleoside linkages that may be used in the EONs of the present disclosure are those that are disclosed in WO2023/278589.

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-OMe, 2′-F, and 2′-MOE modifications of the sugar and the use of PS linkages between nucleosides. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. WO2024/013360 describes the modification of the 2′ position of the ribose sugar moiety of the orphan nucleotide by a 2′,2′-disubstituted substitution such as diF, which is also applicable to the disclosure described here. The 2′-4′ linkage can be selected from many linkers known in the art, such as a methylene linker, amide linker, or constrained ethyl linker (cEt).

The EONs of the present disclosure may also be administered in the context of aids that will increase the entry of the EON into the target cell and/or its endosomal escape as soon as it is in the cell. Moieties that can be applied for such applications are for example a set of chemical compounds (generally purified from nature) referred to as “saponins” or “triterpene glycosides”. A preferred saponin that can be used in the methods of the present disclosure is AG1856, disclosed in WO2021/122998 and further described for use with RNA editing producing oligonucleotides in PCT/EP2024/051278 (unpublished).

The disclosure relates to an EON for use in the deamination of a target A in a target RNA, wherein the EON is complementary to a stretch of nucleotides in the target RNA that includes the target A, wherein the nucleotide in the first nucleic acid strand that is directly opposite the target nucleotide is the orphan nucleotide, and the orphan nucleotide comprises preferably a base or modified base or base analogue with a NH moiety at the position similar to the ring nitrogen (e.g., Benner's base Z).

The nucleotide numbering in the EON is such that the orphan nucleotide is number 0 and the nucleotide 5′ from the orphan nucleotide is number+1. Counting is further positively (+) incremented towards the 5′ end and negatively (−) incremented towards the 3′ end, wherein the first nucleotide 3′ from the orphan nucleotide is number −1.

The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one embodiment, the PS linkages connect the terminal 3, 4, 5, 6, 7, or 8 nucleotides on each end of the first nucleic acid strand. In one embodiment, the EON comprises one of more phosphoramidate (PN) linkages. In one embodiment, a PNdmi linkage connects the terminal two nucleotides on each end of the EON, although either one or both can be changed to a PNms linkage. The same holds true for MP linkages that can be changed to a PNms linkages if such is desired.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds of the disclosure, compounds of the disclosure may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted) alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino) propyl), 2′-O-(2-amino)ethyl, 2′-O-(2-(dimethylamino)ethyl); 2′-deoxy (DNA); 2′-O-(haloalkyl) methyl such as 2′-O-(2-chloroethoxy) methyl (MCEM), 2′-O-(2,2-dichloroethoxy) methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNANC (NH) monomer, a 2′,4′-BNANC (NMe) monomer, a 2′,4′-BNANC (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON herein is complementary to part of the base sequence of a target ANGPTL3 transcription product that includes at least the target A that is to be deaminated to an I, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The EON according to the present disclosure, in contrast to what has been described for siRNA, or gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. It is not preferred that the target transcript molecule is degraded through the binding of the EON to the transcript molecule. In one embodiment, the EON does not comprise four or more consecutive DNA nucleotides anywhere within its sequence. In an embodiment, the EON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON (such as the nucleotide at position −1) may be DNA, but also that there is no stretch of four or more consecutive DNA nucleotides within the EON. Hence, the EON according to the present disclosure is not a gapmer. A gapmer reduces the expression of a target transcript but does not produce RNA editing of a specified adenosine within the target transcript. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON according to the disclosure may be any oligonucleotide that produces an RNA editing effect in which a target A in a target RNA molecule is deaminated to an I, and accordingly is resistant to RNase-mediated breakdown as much as possible to yield this effect.

In one embodiment, the EON, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations).

Examples of a cleavable linker comprise, but are not limited to, an amide, an ester, one or both esters of a PO, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, TEG, HEG, alkyl chains, propyl, 6-aminohexyl, or dodecyl. In one embodiment, a GalNAc moiety is bound to the EON of the present disclosure via a TEG linker.

The disclosure also relates to a pharmaceutical composition comprising the EON according to the disclosure, and further comprising a pharmaceutically acceptable carrier and/or other additive (such as a saponin or triterpene glycoside like AG1856 (as discussed above), which in fact may also be administered separately from the EON) and may be dissolved in a pharmaceutically acceptable organic solvent, or the like. The EON of the present disclosure may also be conjugated to the AG1856 saponin, which would allow very efficient endosomal release after cell entry. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one embodiment, the EON comprises at least one nucleotide with a sugar moiety that comprises a 2′-OMe modification. In one embodiment, the EON comprises at least one nucleotide with a sugar moiety that comprises a 2′-MOE modification. In one embodiment, the EON comprises at least one nucleotide with a sugar moiety that comprises a 2′-F modification. In one embodiment, the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the EON is an antisense oligonucleotide that can form a double stranded nucleic acid complex with a target RNA molecule, wherein the double stranded nucleic acid complex can recruit an adenosine deaminating enzyme for deamination of a target adenosine in the target ANGPTL3 RNA molecule, wherein the nucleotide in the EON that is opposite the target adenosine is the orphan nucleotide, and wherein the orphan nucleotide has the structure of formula (IV):

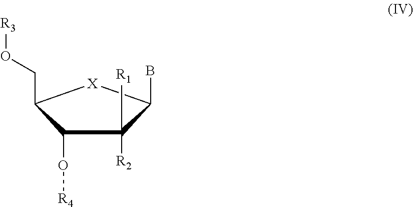

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-yl-2 (1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position −1).

In one embodiment, the EON comprises at least one nucleotide with a sugar moiety that comprises a 2′-F modification. A preferred position for the nucleotide that carries a 2′-F modification is position −3 in EON, which may be present together with an identical 2′ modification in the orphan nucleotide as discussed above.

In one embodiment, the EON comprises at least one phosphonoacetate or phosphonoacetamide internucleoside linkage.

In one embodiment, the EON comprises at least one nucleotide comprising a locked nucleic acid (LNA) ribose modification, or an unlocked nucleic acid (UNA) ribose modification. In an embodiment, the EON comprises at least one nucleotide comprising a threose nucleic acid (TNA) ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON of the present disclosure are disclosed in WO2020/154342, WO2020/154343, and WO2020/154344.

In one embodiment, the EON of the present disclosure may comprise one or more nucleotides carrying a 2′-MOE ribose modification. Also, in one embodiment, the EON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the EON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the EON comprises one or more nucleotides comprising a 2′ position comprising a 2′-MOE, 2′-OMe, 2′-OH, 2′-H, TNA, 2′-F, 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g. WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the EON fulfils its role as an editing producing oligonucleotide that can form a double stranded complex with the target RNA and recruit a deaminating enzyme, that can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

The nucleobases in an EON of the present disclosure can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms PO bonds. The PO and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because PO bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs according to the disclosure can comprise linkage modifications. A linkage modification can be, but not limited to, a modified version of the PO present in RNA, such as PS, chirally pure PS, (R)-PS, (S)-PS, methyl phosphonate (MP), chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′->3′ and 2′>5′ linkages.

In one embodiment, an EON comprises a substitution of one of the non-bridging oxygens in the PO linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modifications of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise EONs as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence recruit an adenosine deaminase enzyme because of the dsRNA nature that arises. In all aspects of the disclosure, the enzyme with adenosine deaminase activity is preferably ADAR1 or ADAR2. In a highly preferred embodiment, the EON is an RNA editing oligonucleotide that targets a pre-mRNA or an mRNA, wherein the target nucleotide is an A in the target RNA, wherein the A is deaminated to an I, which is being read as a G by the translation machinery. The disclosure also relates to a pharmaceutical composition comprising the EON as characterized herein, and a pharmaceutically acceptable carrier.

Other chemical modifications of the EON according to the disclosure include the substitution of one or more than one of any of the hydrogen atoms with deuterium or tritium, examples of which can be found in e.g., WO2014/022566 or WO2015/011694.

The disclosure relates to an EON according to the disclosure, or a pharmaceutical composition comprising an EON according to the disclosure, for use in the treatment, amelioration, or prevention of a disorder related to high or increased or unwanted levels of LDL-C and/or triglycerides in the plasma. In one embodiment, the disclosure relates to an EON according to the disclosure, or a pharmaceutical composition comprising an EON according to the disclosure, for use in the treatment, amelioration, or prevention of a disorder related to high or increased or unwanted levels of LDL-C and/or triglycerides in the plasma. In one embodiment, the disclosure relates to an EON according to the disclosure, or a pharmaceutical composition comprising an EON according to the disclosure, for use in the treatment, amelioration, or prevention of a disorder related to high or increased or unwanted levels of LDL-C and/or triglycerides in the plasma, such as ASCVD or similar cardiovascular diseases.

EONs of the present disclosure preferably do not include a 5′-terminal 06-benzylguanosine or a 5′-terminal amino modification and preferably are not covalently linked to a SNAP-tag domain (an engineered 06-alkylguanosine-DNA-alkyl transferase). EONs of the present disclosure preferably do not comprise a boxB RNA hairpin sequence. In one embodiment, an EON of the present disclosure comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is U. One alternative for U is positioning an isoU opposite the target adenosine, which likely does not pair like G pairs with U. In one embodiment, the target A in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. Although it is preferred to use ‘naked’ EONs that have chemical modifications as outlined herein, EONs that are delivered through other means, for instance through AAV vector expression, or editing molecules that are circular, or have hairpin structures (recruiting portions, e.g., as disclosed in WO2016/097212, WO2017/050306, WO2020/001793, WO2017/010556, WO2020/246560, and WO2022/078995) are also encompassed by the present disclosure because these can also be applied to edit A's in the target ANGPTL3 RNA molecule to generate an ANGPTL3 protein with diminished or absent lipase inhibitory function. An EON that is ‘packaged’ in a delivery vehicle, such as a lipid nanoparticle (LNP), is still considered ‘naked’, as it is not transcribed from a coding sequence, but manufactured in a manufacturing facility. After such manufacturing, the chemically modified EON can then be further processed to be encapsulated by a delivery vehicle such as an LNP. LNP's that can be used to deliver EONs as disclosed herein can be any type of LNP known in the art. Hence, the disclosure also relates to a delivery vehicle, preferably an LNP, which comprises a ‘naked’, and preferably chemically modified EON as disclosed herein, even more preferably as disclosed in any one of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, and 29.

An EON according to the present disclosure can utilise endogenous cellular pathways and naturally available ADAR enzymes to specifically edit a target A in the target RNA sequence. An EON of the disclosure is capable of recruiting ADAR and complex with it and then facilitates the deamination of a (single) specific target A in a target RNA sequence. Ideally, only one A is deaminated. An EON of the disclosure, when complexed to ADAR, preferably brings about the deamination of a single target A.

Analysis of natural targets of ADAR enzymes has indicated that these generally include mismatches between the two strands that form the RNA helix edited by ADAR1 or ADAR2. It has been suggested that these mismatches enhance the specificity of the editing reaction (Stefl et al. 2006. Structure 14 (2): 345-355; Tian et al. 2011. Nucleic Acids Res 39 (13): 5669-5681). Characterization of optimal patterns of paired/mismatched nucleotides between the EONs and the target RNA also appears important to the development of efficient ADAR-based EON therapy.

As outlined above, an EON of the present disclosure makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the PO linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′-OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42 (10): e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. The disclosure relates to RNA editing oligonucleotides, generally referred to as EONs herein, that can bring about deamination of an A in the ANGPTL3 transcript, with a resulting ANGPTL3 protein that has lost or is diminished in its function to inhibit lipases. This means that the disclosure is not strictly limited to deamination of the A of wild-type ANGPTL3, but that other (single or multiple) A's may be targeted, which may also result in diminished ANGPTL3 protein function. Other A's may be identified, for instance by genetic screening in the population, or in silico, that are also important (or may become more important) for ANGPTL3 function, and that also may be targeted through RNA editing, following the teaching of the present disclosure. All such RNA events and oligonucleotides that can be used for such targeting are encompassed by the present disclosure, no matter what the exact nucleic molecule, or EON, looks like.

Mutagenesis studies of human ADAR2 revealed that a single mutation at residue 488 from glutamic acid to glutamine (E488Q), gave an increase in the rate constant of deamination by 60-fold when compared to the wild-type enzyme (Kuttan and Bass. Proc Natl Acad Sci USA 2012. 109 (48): 3295-3304). During the deamination reaction, ADAR flips the edited base out of its RNA duplex, and into the enzyme active site. When ADAR2 edits A's in the preferred context (an A:C mismatch) the nucleotide opposite the target A is often referred to as the ‘orphan cytidine’, as indicated above. The crystal structure of ADAR2 E488Q bound to double stranded RNA (dsRNA) revealed that the glutamine (Gln; Q) side chain at position 488 can donate an H-bond to the N3 position of the orphan C, which leads to the increased catalytic rate of ADAR2 E488Q. In the wild-type enzyme, wherein a glutamic acid (Glu; E) is present at position 488 instead of a glutamine (Gln) the amide group of the glutamine is absent and is instead a carboxylic acid. To obtain the same contact of the orphan C with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of EONs with modified RNA bases, especially at the position of the orphan C to mimic the hydrogen-bonding pattern observed by the E488Q ADAR2 mutant. By replacing the nucleotide opposite the target A in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem. 2009. 74 (21): 8021-8030; Burchenal et al. 1976. Cancer Res 36:1520-1523) and Benner's base Z (also referred to as ‘dZ’; Yang et al. Nucl Acid Res 2006. 34 (21): 6095-6101) that were initially selected because they offer hydrogen-bond donation at N3 with minimal perturbation to the shape of the nucleobase. Benner's base is also referred to as 6-amino-5-nitro-3-yl-2 (1H)-pyridone. The presence of the cytidine analog in the EON may exist in addition to modifications to the ribose 2′ group. The ribose 2′ groups in the EON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, a nucleotide analogue or equivalent within the EON comprises one or more base modifications or substitutions. Modified bases comprise synthetic and natural bases such as inosine, xanthine, hypoxanthine and other-aza, deaza,-hydroxy,-halo,-thio, thiol,-alkyl, -alkenyl,-alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON according to the disclosure is normally longer than 10 nucleotides, preferably more than 11, 12, 13, 14, 15, 16, still more preferably more than 17 nucleotides. In one aspect the EON according to the disclosure is longer than 20 nucleotides. The oligonucleotide according to the disclosure is preferably shorter than 100 nucleotides, still more preferably shorter than 60 nucleotides, still more preferably shorter than 50 nucleotides. In a preferred aspect, the oligonucleotide according to the disclosure comprises 18 to 70 nucleotides, more preferably comprises 18 to 60 nucleotides, and even more preferably comprises 18 to 50 nucleotides. Hence, in a particularly preferred aspect, the oligonucleotide of the present disclosure comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides. In one embodiment, the EON is 27, 28, 29, or 30 nucleotides in length. In one embodiment, the design is a-symmetrical such that the number of nucleotides 5′ from the orphan nucleotide is 22, 23, or 24, and the number of nucleotides 3′ from the orphan nucleotide is 5, 6, or 7.

As described above, in some embodiments the disclosure provides an EON for forming a double stranded complex with a human ANGPTL3 RNA molecule in a human liver cell. Thus, the therapeutic effect is preferably on a human liver cell in vivo. Of course, the methods may also be carried out in vitro or ex vivo.

The disclosure provides an EON of the disclosure, or pharmaceutical composition of the disclosure, for use in the treatment of disease. The disclosure also provides the use of an EON of the disclosure, or pharmaceutical composition of the disclosure, in the manufacture of a medicament for the treatment of disease. The disclosure also provides a method for treating a disease in a patient, comprising administering a therapeutically effective amount of an EON according to the disclosure or a pharmaceutical composition according to the disclosure. Preferably the disease is a disease caused by high (or increased) levels of LDL-C and/or triglycerides. The EON is administered therapeutically or prophylactically because both types of treatment could be beneficial.

After RNA editing has occurred in a cell, the modified RNA can become diluted over time, for example due to cell division, limited half-life of the edited RNAs, etc. Thus, in practical therapeutic terms a method of the disclosure may involve repeated delivery of an EON until enough target RNAs have been modified to provide a tangible benefit to the patient and/or to maintain the benefits over time.

### EXAMPLES

### Example 1. Editing of a Target Adenosine in a Human ANGPTL3 Target RNA Molecule in Cells

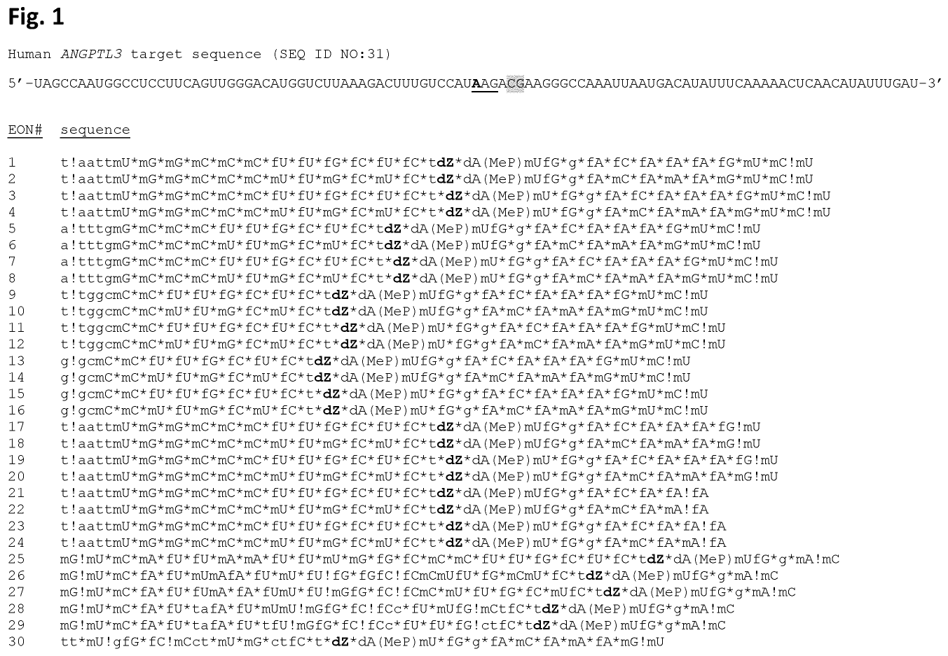

An initial set of 30 ANGPTL3-targeting EONs (see FIG. 1, wherein EON #1 to #30 are RM5035 to RM5064, respectively) were tested to assess whether editing of human ANGPTL3 target (pre-) mRNA in cells was feasible after gymnotic incubation with the EONs (gymnotic=without any transfection means).

### Cell Culture

Human Huh-7 hepatocyte-derived cellular carcinoma cells (CLS Cell Lines Service GmbH) were cultured in RPMI 1640 supplemented with 10% FBS/2 mM L-glutamine and kept at 37° C. in a 5% CO₂ atmosphere. Primary human hepatocytes (PHH's; BiolVT) were cultured in complementary INVITROGRO Plating and Maintenance Medium (BiolVT) and kept at 37° C. in a 10% CO₂ atmosphere. PHH-derived liver-spheroids were generated using the manufacturer's (BiolVT) protocol.

### Gymnotic Exposure

A total of 0.5×10⁵ Huh-7 cells were seeded in wells of a 24-well plate one day prior exposure to the EONs. After ON incubation, plating medium was aspirated and mixtures of EON in fresh culture medium were added to the cells. In experiments where the saponin (triterpene glycoside; see WO2021/122998) AG1856 was added, the mixture containing 1 μM EON was supplemented with 1 μM AG1856. Cells were incubated with the inoculates for 72-hrs before total RNA isolation.

For the gymnotic treatment of the human primary hepatocyte derived spheroids, 1.5×10³ cells were seeded in a 96-well plate in a total volume of 100 μL plating medium. Plates were subjected to 2 min of 250 g centrifugation to accumulate the cells to the bottom of the well. Spheroids were formed over 5 days of incubation. Before exposure, medium was aspirated and mixtures containing 5 μM EON and 1 μM AG1856 in fresh maintenance medium were added. After 72 hrs incubation, the medium was aspirated, and total RNA was isolated.

### Total RNA Isolation

72 hours post exposure to the EONs, cells were collected, and total RNA was isolated from the cells using the Direct-zol RNA Microprep kit (Zymo Research). After removal of the culture medium, the cells were washed once with PBS. After complete aspiration of the PBS, 100 UL TRIreagent (Zymo Research) was added to lyse the cells and collect the intracellular material. For the spheroids, 300 μL of TRIreagent was used. After addition of 100 μL ethanol (300 μL for the spheroid samples), the mixtures were loaded in a column and subjected to several wash steps and DNasel treatment. After elution in a total volume of 15 μL DNase/RNase-free water, the RNA yield was determined using spectrophotometric analysis (NanoDrop) and stored at −80° C.

cDNA Synthesis

Maxima Reverse Transcriptase (RT, ThermoFisher) was used to generate cDNA. Typically, 100 nanogram total RNA was used in a reaction mixture containing 4 μL 5x RT buffer, 1 μL dNTP mix (10 mM each), 0.5 μL Oligo (dT), 0.5 μL random hexamer (all ThermoFisher) supplemented with DNase- and RNase-free water to a total volume of 20 μL. Samples were loaded in a T100 thermocycler (Bio-Rad) and initially incubated at 10 min at 25° C., followed by a cDNA reaction temperature of 50° C. (30 min) and a termination step of 5 min at 85° C. Samples were cooled down to 4° C. prior storing at −20° C.

dPCR Assays

To determine the editing efficiency, cDNA samples were used in multiplex dPCR (Qiagen) assays. The first assay was designed to distinguish between cDNA species containing the original A or the edited I (which is converted into a G during cDNA synthesis and subsequent PCR). The second multiplex ddPCR quantifies the amount of ANGPTL3 transcripts measuring exon 6-7 specific fragments. A separate HPRT1-specific dPCR was used to correct for variation in sample isolation or possible effects during exposure, using a HPRT1-specific primer/probe set. The primer and probe sequences are listed in Table 1, the cycling conditions in Table 2.

In total, 1 μL of the cDNA mix was used in a dPCR mixture containing 3 μL 4x dPCR QIAcuity Mastermix for probes (Qiagen), 0.6 μL primers and 0.3 μL probes (10 μM stock concentration each), supplemented with 4.5 μL DNase- and RNase-free water in a total volume of 12 μL. The resulting mixture was mixed thoroughly and transferred to a well of a QIAcuity 96-wells 8.5K Nanoplate (Qiagen) and loaded in a QIAcuity dPCR machine. Data was analyzed using the QIAcuity Software Suit (Qiagen). Percentage of A-to-I editing was determined by dividing the number of G-containing molecules by the total (G-plus A-containing species) multiplied by 100.

### Results

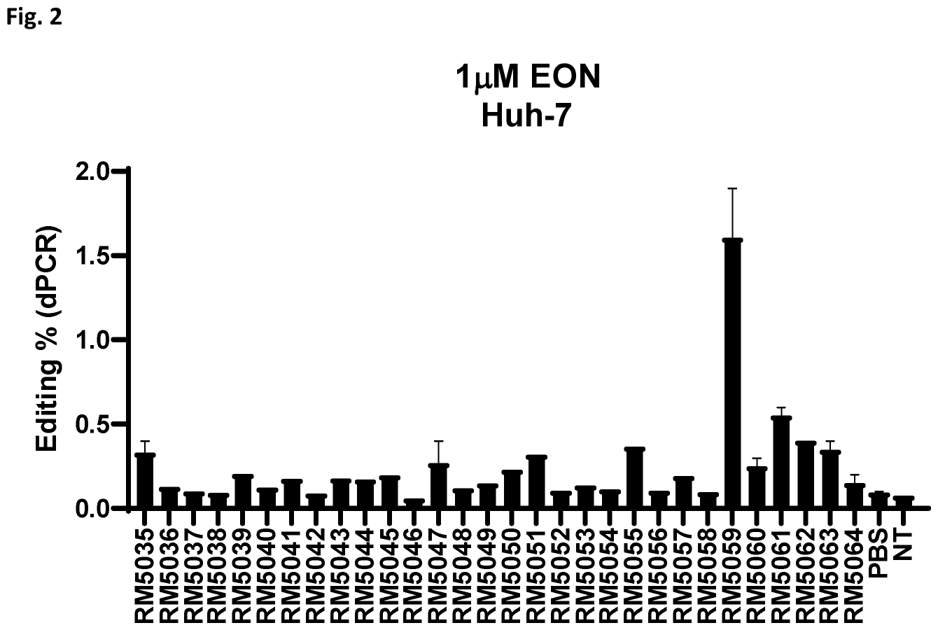

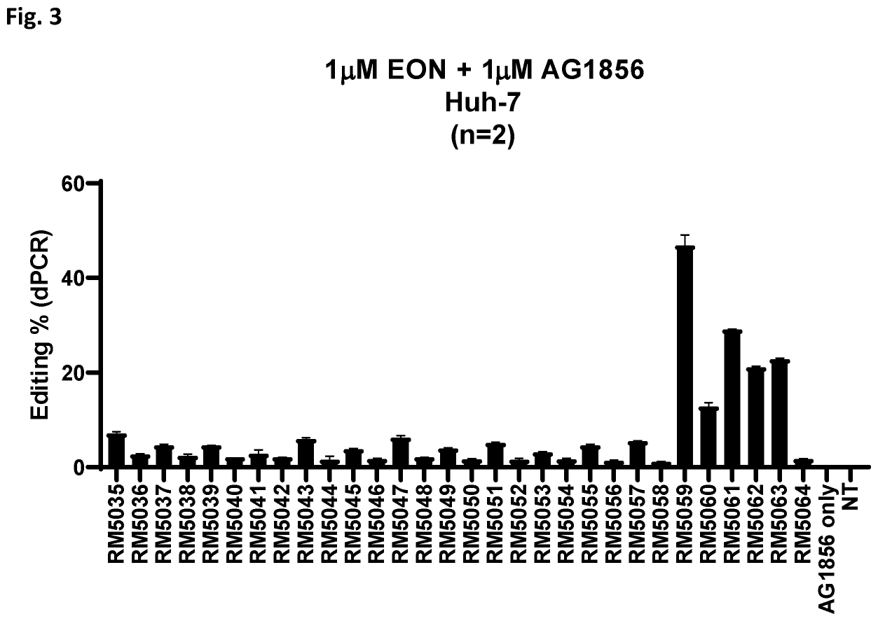

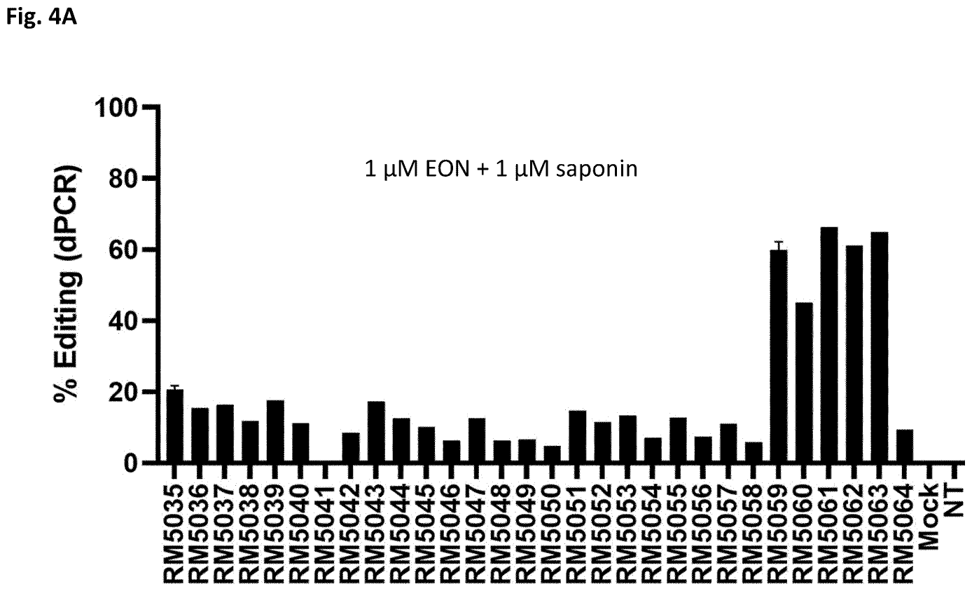

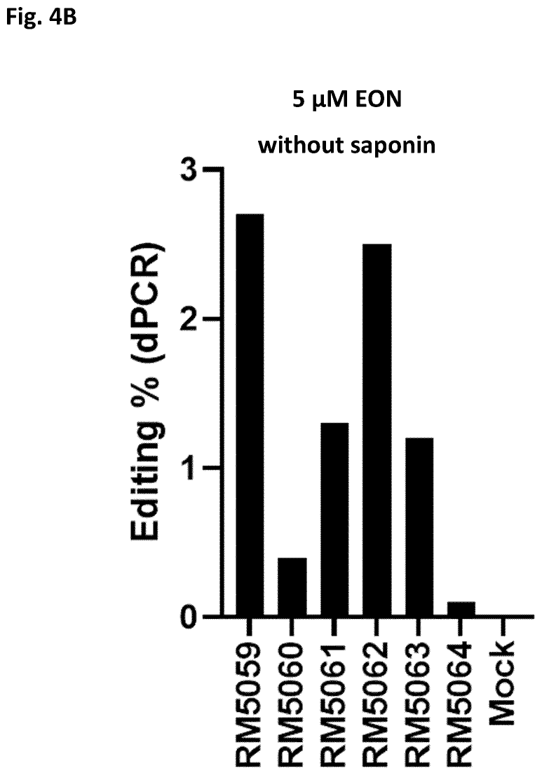

FIG. 2 shows the percentage A to I editing as determined in human Huh-7 cells after incubation with the 30 indicated EONs using a gymnotic approach in which no transfection agents were applied, as outlined above. Although some EONs showed hardly any activity above NT and PBS negative controls, clear editing could be observed in many of the treated samples, with RM5059 (EON #25 in FIG. 1) giving the highest percentage. FIG. 3 shows the results of an identical experiment, but wherein the Huh-7 cells were co-incubated with the triterpene glycoside AG1856. Clearly, no editing could be observed in the non-treated sample and the AG1856 alone control, whereas all EONs showed editing above background, with RM5059 performing best, like what was found in the experiment without AG1856. FIG. 4A shows the results of the experiment in which the same 30 EONs were tested for A to I editing of ANGPTL3 transcripts in liver spheroids, generated from PHH's. All incubations of EONs were accompanied by incubation with 1 μM AG1856, and all EONs (except for RM5041, which likely resembles an experimental error because editing was found for this EON in the Huh-7 cells) showed very significant levels of editing, with RM5059 again performing good together with RM5061, RM5062, and RM5063, with editing percentages of more than 60% of the ANGPTL3 transcripts. The same experiment was performed in liver spheroids generated from PHH's using a 5 μM incubation of six selected EONs: RM5059 (EON #25), RM5060 (EON #26), RM5061 (EON #27), RM5062 (EON #28), RM5063 (EON #29), and RM5064 (EON #30), but without the addition of the saponin. The results are shown in FIG. 4B and indicate that RNA editing of the target adenosine in the endogenous ANGPTL3 transcript could be achieved also in the absence of the saponin, with a similar pattern across the different oligonucleotides.

TABLE 1
Primers and probes

| Name | Sequence 5′-3′ |
|---|---|
| hANGPTL3_e01_Fw1 | AATGGCCTCCTTCAGTTGGG (SEQ ID NO: 32) |
| hANGPTL3_e01_Rv1 | TGATTTCACTGGTTTGCAGCG (SEQ ID NO: 33) |
| hANGPTL3_e01_FAM | CTTTGTCCA+T+G+AGACGAAG (SEQ ID NO: 34) |
| hANGPTL3_e01_HEX | CTTTGTCCA+T+A+AGACGAAGG (SEQ ID NO: 35) |
| hANGPTL3_e06_Fw1 | CGATTACTGGCAATGTCCCC (SEQ ID NO: 36) |
| hANGPTL3_e07_Rv1 | TCTCCACACTCATCATGCCAC (SEQ ID NO: 37) |
| hANGPTL3_e06_TEX | ACTTCAACTGTCCAGAGGGT (SEQ ID NO: 38) |
| HPRT1 fw | CCTGGCGTCGTGATTAGTGA (SEQ ID NO: 39) |
| HPRT1 rv | CGAGCAAGACGTTCAGTCCT (SEQ ID NO: 40) |
| HPRT1 TEX red | TGC+TGAGGATTTGGAAAGGGTGT (SEQ ID NO: 41) |

TABLE 2
ddPCR cycling conditions.

| Step | Temperature | Time | Cycles |
|---|---|---|---|
| Enzyme activation | 95 °C | 2 minutes | 1 |
| Denaturation | 95 °C | 15 seconds | 40 |
| Annealing/extension | 60 °C | 30 seconds | |
| Hold | 4 °C | Indef. | ∞ |

### Example 2. Editing of a Target Adenosine in a Human ANGPTL3 Target RNA Molecule in PHH's

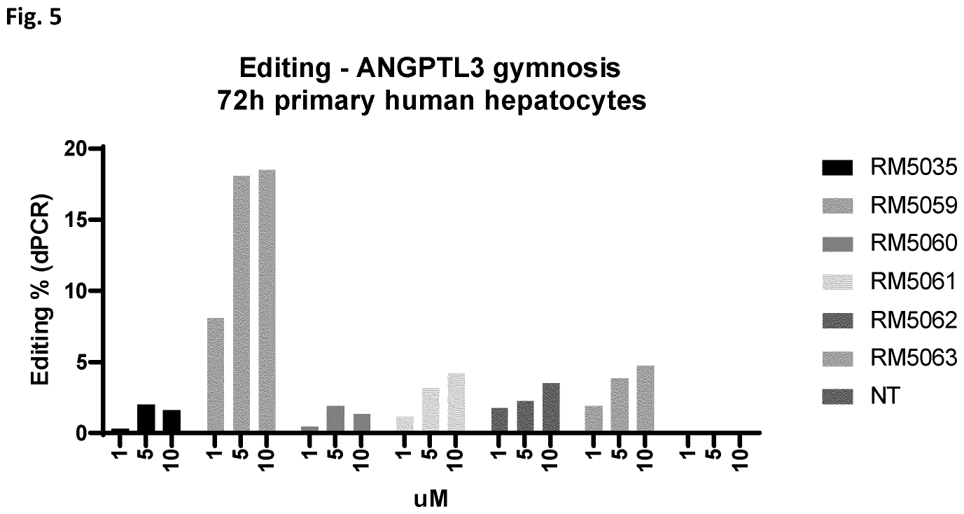

A similar experiment as outlined in Example 1 was performed in PHH's. Cells were seeded on day in 96-wells plates and on day 1 incubated with either 1, 5, or 10 μM EON. The EONs that were tested were RM5035, RM5059, RM5060, RM5061, RM5062, and RM5063 (EONs #1, #25, #26, #27, #28, and #29, respectively, see FIG. 1). No saponin was added to the culture medium, and 72 hrs later cells were harvested, and used for dPCR analysis as described above. FIG. 5 shows the results from these gymnotic uptake experiments in PHH's and percentages of RNA editing in these cells. Clearly, RM5059 outperformed the other EONs in all three concentrations tested, with editing percentages up to 20% after incubation with 5 and 10 μM EON, even though significant levels of editing could also be observed with the other EONs that were used in the experiment.

### Example 3. Editing of a Target Adenosine in a Human ANGPTL3 Target RNA Molecule in Liver Spheroids Using GaINAc-Conjugated Oligonucleotides

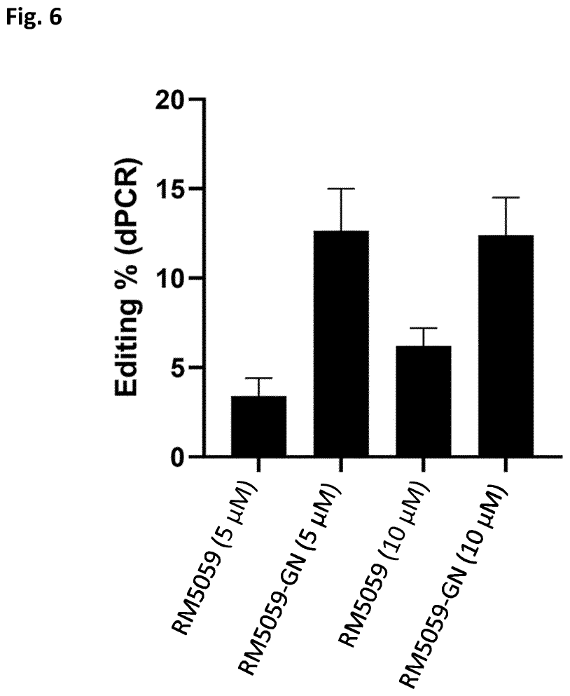

Next, it was tested whether the addition of a GalNAc moiety, attached to the 5′ terminus of the oligonucleotide could beneficially influence the efficiency of RNA editing. For this, liver spheroids, generated from PHH's, as outlined above, were used. These were incubated with 5 or 10 μM RM5059 or GaINAc-RM5059 (EON #25 and EON #55, respectively, see legends to FIG. 1) as outlined above, without any saponin (gymnotic uptake only), and subsequently used for dPCR to address ANGPTL3 transcript editing. The results are shown in FIG. 6 and indicate that the addition of the GalNAc moiety to the 5′ terminus of RM5059 significantly contributed to the RNA editing efficiency in these liver spheroids (2- to 3-fold increase).

### Example 4. ANGPTL3 Binding to Heparin after RNA Editing of Endogenous Human ANGPTL3 Target RNA in Huh-7 Hepatocyte Cells

To determine whether editing of the ANGPTL3 transcript at the position that encodes the heparin binding site in the ANGPTL3 protein results in a diminished ability to bind heparin, Huh-7 hepatocyte cells were gymnotically treated with 1 μM RM5059, RM5035, and RM5047 (EON #25, EON #1, and EON #13 in FIG. 1, respectively) together with 1 μM AG1856 for 3 days. Cells treated with saponin-only were taken along as negative controls. After incubation with the EONs, cells were lysed in RIPA buffer. It was tested whether EON treatment affected ANGPTL3 transcript levels, which turned out to be not the case (data not shown).

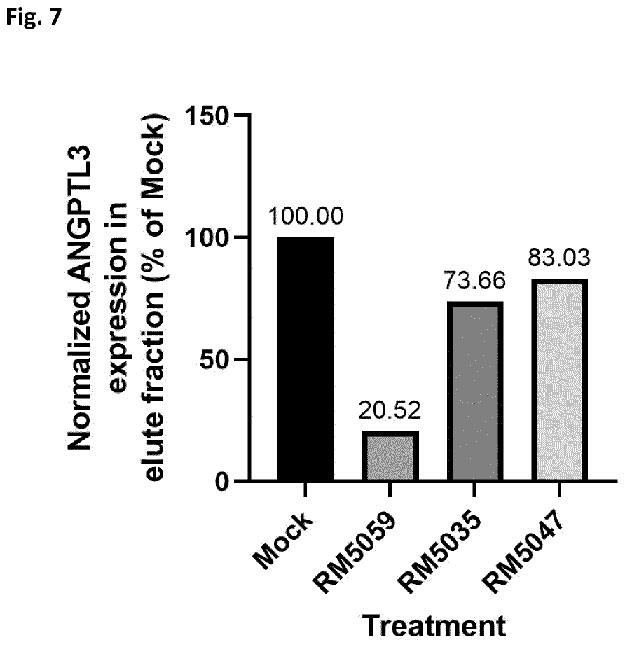

If there would be less ANGPTL3 protein capable of heparin binding due to the editing of the encoding transcript RNA, as outlined herein, it should be feasible to test this by having proteins bind to a heparin column and then determine how much ANGPTL3 would bind and could be eluted from such columns. After run-through of the lysates, it was envisioned that EON treatment should result in lower amounts of ANGPTL3 protein getting stuck in the heparin columns, even though same amounts of ANGPTL3 proteins were produced. The eluted protein sample was run on a western blot, then stained with an ANGPTL3-specific antibody, and intensities of the ANGPTL3 protein on the western blot were subsequently examined. For this, cell lysates from the treated Huh-7 cells were first run over heparin-Sepharose columns (HiTrap), and bound proteins were subsequently eluted and examined. A rabbit anti-ANGPTL3 polyclonal serum was used for the western blot, while an anti-vinculin antibody was taken along to normalize for protein loading. The signals obtained at the size of the human ANGPTL3 protein indicated a lower amount of ANGPTL3 protein that was eluted from the columns in the oligonucleotide-treated samples (western blot data not shown). These signals were normalized against the vinculin signal and results are provided in FIG. 7, clearly showing a significantly lower signal of heparin-bound ANGPTL3 protein obtained from the heparin columns that received cell lysates from RM5059-RM5035-, and RM5047-treated Huh-7 cells. This clearly is indicative that the RNA editing mediated by these oligonucleotides resulted in ANGPTL3 protein that was less capable of binding heparin, hence showing a functional effect of the RNA editing at the protein level.

These results together show that the inventors were able to achieve very specific and very high RNA editing of endogenous ANGPTL3 transcripts in human liver cells, more specifically of the first A of the codon in human ANGPTL3 pre-mRNA or mRNA that codes for amino acid 63 in the human ANGPTL3 protein. Moreover, the data shows that the editing that was achieved in Huh-7 cells resulted subsequently in the appearance of ANGPTL3 protein that was less able to bind heparin in heparin columns. This shows that the RNA editing mediated by treatment with oligonucleotides directly resulted in a desired loss-of-function effect in the translated human ANGPTL3 protein from the edited ANGPTL3 transcripts.

### Example 5. Editing of a Target Adenosine in a Mouse Angptl3 Target RNA Molecule In Vivo

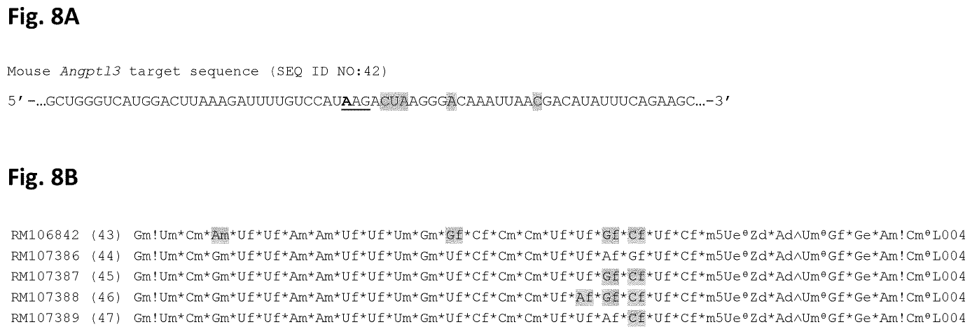

In a next experiment, EONs were tested in vivo for editing the mouse Angptl3 target sequence corresponding to the human sequence. Part of the mouse Angptl3 transcript is provided in FIG. 8A, in which the AAG codon (equivalent to the human codon AAG that encodes lysine at position 63 in the human protein) is shown with the target adenosine in bold. Since there are several differences between the human target sequence and the mouse sequence (positions shown in grey boxes in FIG. 8A), a new set of EONs was designed to be used in mice to investigate in vivo editing. Initially, five different EONs were designed, of which the sequence and chemical modifications are shown in FIG. 8B. Each of these EONs were linked to a 3′ terminally located tri-antennary GalNAc moiety. Notably, RM107387 (SEQ ID NO:45) comprises nucleotides on position +4 and +5 that mismatch with the target sequence (in mouse), similar to the two mismatches that are present in RM5059 (EON #25) in respect to the target human sequence, although RM5059 has a C:C mismatch at +4 and a G:G mismatch at +5, and RM107387 has a C:C mismatch at +4 and a G: U mismatch at +5. The remainder of RM107387 is complementary to the mouse target sequence.

The experimental setup was as shown in table 3, in which the EONs or controls were administered subcutaneously (SC), with three doses: the first on Day 0, the second on Day 2, and the third on Day 3. Mice were either sacrificed on day 7 or day 14 after start of the study. The amount of EON per dose was 50 mg/kg (PBS control=0). All groups contained 4 mice.

TABLE 3
Experimental setup in vivo study

| Group | EON | Necropsy |
|---|---|---|
| 1A | PBS | Day 7 |
| 1B | PBS | Day 14 |
| 2A | RM106842 | Day 7 |
| 2B | RM106842 | Day 14 |
| 3A | RM107387 | Day 7 |
| 3B | RM107387 | Day 14 |
| 4A | RM107389 | Day 7 |
| 4B | RM107389 | Day 14 |
| 5A | RM107387 | Day 7 |
| 5B | RM107387 | Day 14 |
| 6A | RM107388 | Day 7 |
| 6B | RM107388 | Day 14 |

On the day of sacrifice, RNA was isolated from liver and kidney using standard procedures known in the art. Further cDNA production and dPCR analysis for editing were performed generally as described in example 1, with the mouse target-specific primers and probes of Table 4.

TABLE 4
Primers and probes

| Name | Sequence 5′-3′ |
|---|---|
| mANGPTL3_ex1_FW1 | TTTTAGCGAATGGCCTCCTG (SEQ ID NO: 48) |
| mANGPTL3_ex1_REV | CATTGGTTCGAAGTGATAGGTC (SEQ ID NO: 49) |
| mANGPTL3_ex1_ori_FAM | /56-FAM/TTTTGTCCA+T+A+AGACTAAGGGACA/3BHQ_1/ (SEQ ID NO: 50) |
| mANGPTL3_ex1_edit_HEX | /5HEX/TTTTGTCCA+T+G+AGACTAAGGGA/3BHQ_1/ (SEQ ID NO: 51) |
| mANGPTL3_ex6_FW1 | AGATTGCTGGCAATATCCCT (SEQ ID NO: 52) |
| mANGPTL3_ex7_REV1 | TCTCCACATATGTCATTCCAC (SEQ ID NO: 53) |
| mANGPTL3_ex6_ref_TEX | /5TEX615/AGCTC+T+ACTGT+CCAGAAAGT/3IAbRQSp/ (SEQ ID NO: 54) |

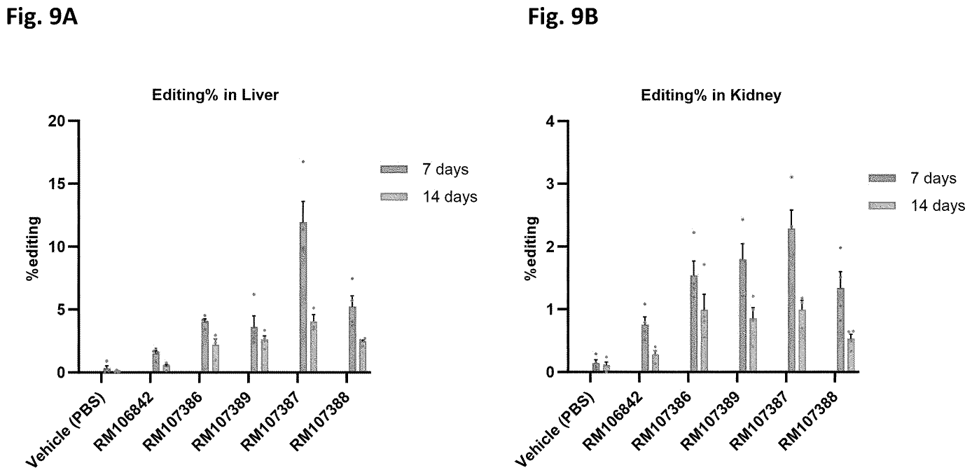

FIG. 9A shows the percentage editing in liver at day 7 (left bars) and day 14 (right bars) as indicated. FIG. 9B shows the percentage editing in kidney at day 7 (left bars) and day 14 (right bars) as indicated. In both cases the editing was highest after use of RM107387 (SEQ ID NO:45), with editing percentages going up to 15% in the liver at day 7.

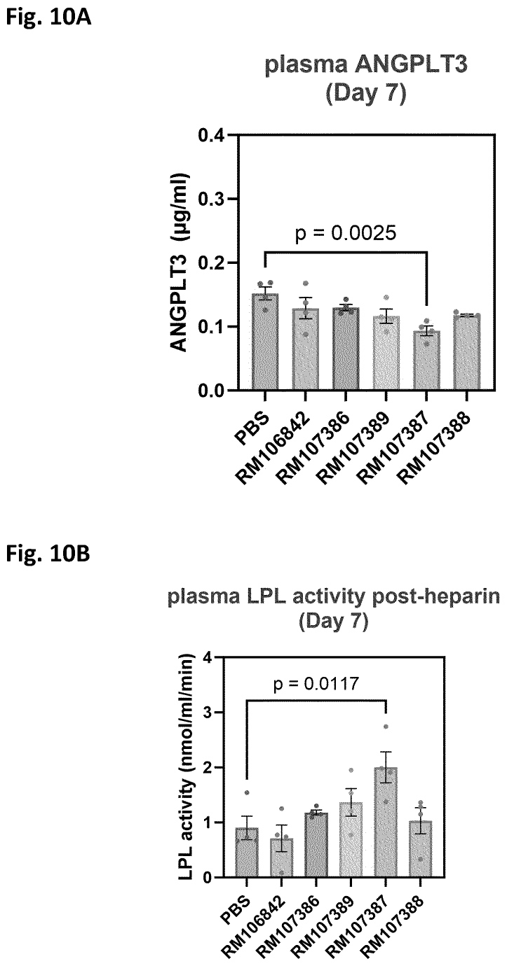

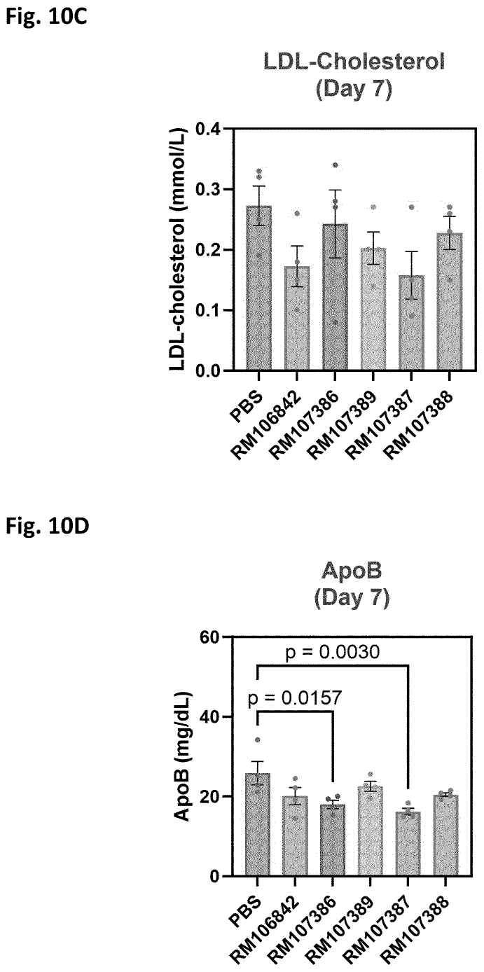

To determine the effect of EON administration to the mice and the effect on Angptl3 protein content (and activity), the concentration of mouse Angptl3 protein in the plasma was determined using the Quantikine mouse ANGPTL3 ELISA from R&D (Novi, MI, USA), applying the protocol of the manufacturer. FIG. 10A shows that at day 7 some decrease in Angptl3 protein content in the plasma could be detected in all EON-treated mice, in comparison to the PBS control. The Angpt13 protein is one of the proteins involved in the regulation of LPL activity, as outlined supra. Hence, LPL activities in plasma were also addressed (which should go up with decreased Angptl3 activity) in the mice treated with the different EONs. Pre- and post-heparin plasma triacylglycerol hydrolase activity was determined in the presence or absence of 1 mol/L NaCl. In the presence of 1 mol/L NaCl, only HL activity is measured. Post-heparin plasma was incubated with 0.2 ml of TG substrate mixture containing triolein (4.6 mg/ml) and [3H]-TO (2.5 uCi/ml) for 30 min at 37° C. in the presence or absence of 1M NaCl, which completely inhibits LPL activity, to estimate both the HL and LPL activity. The LPL activity was calculated as the fraction of total triacylglycerol hydrolase activity that was inhibited by the presence of 1M NaCl and is expressed as the amount of FFAs released per hour per ml of plasma. LPL activity is calculated as the portion of total lipase activity inhibited by 1 mol/L NaCl. FIG. 10B shows that the LPL activity was significantly increased in the plasma samples of the EON-treated mice, with the highest increase in the mice treated with RM107387, that also showed the highest editing in liver (see above). To further investigate the effect of these increased LPL activities on the downstream biomarkers LDL-C, also known as “bad” cholesterol, and Apolipoprotein (ApoB, commonly used to detect risk of atherosclerotic cardiovascular disease), these levels were measured also. For this, the so-called ‘Friedewaldformula’ was used that says: LDL-cholesterol=Total cholesterol-HDL-cholesterol-(0,45×Triglycerides). Total cholesterol levels and plasma HDL-cholesterol levels were determined using the ‘Cholesterol Gen. 2’ kit from Roche (Mannheim, Germany) after the precipitation of ApoB containing lipids by using PEG-6000/glycine. Triglyceride levels were determined using the ‘Trigl’ kit from Roche (Mannheim, Germany). Mouse ApoB levels were determined using the ‘mouse ApoB ELISA’ of Abcam (Cambridge, UK). FIG. 10C shows the LDL-C levels that were determined using the methods above, and FIG. 10D shows the ApoB levels using the methods above. Notably, following the pattern observed with the different levels of editing in the liver after administering the EONs of FIG. 8B (with RM107387 giving the highest percentage of editing) LPL activities went up considerably, also with LPL levels being highest in the samples with the highest level of editing (RM107387). The increased LPL activity, resulting from the editing of the Angptl3 transcript, also appears to affect the levels of LPL-C and ApoB that, as mentioned above, are direct and commonly used biomarkers for CVD. This in vivo experiment shows that the inventors could obtain RNA editing in mice, in the liver, with a direct downstream effect on relevant CVD biomarkers, indicating that ANGPTL3 transcript editing as outlined in detail herein, provides a useful tool in the fight against cardiovascular disorders.

### Example 6. Editing of a Target Adenosine in a Human ANGPTL3 Target RNA Molecule Using EONs with Different 2′-MOE-PO Positions

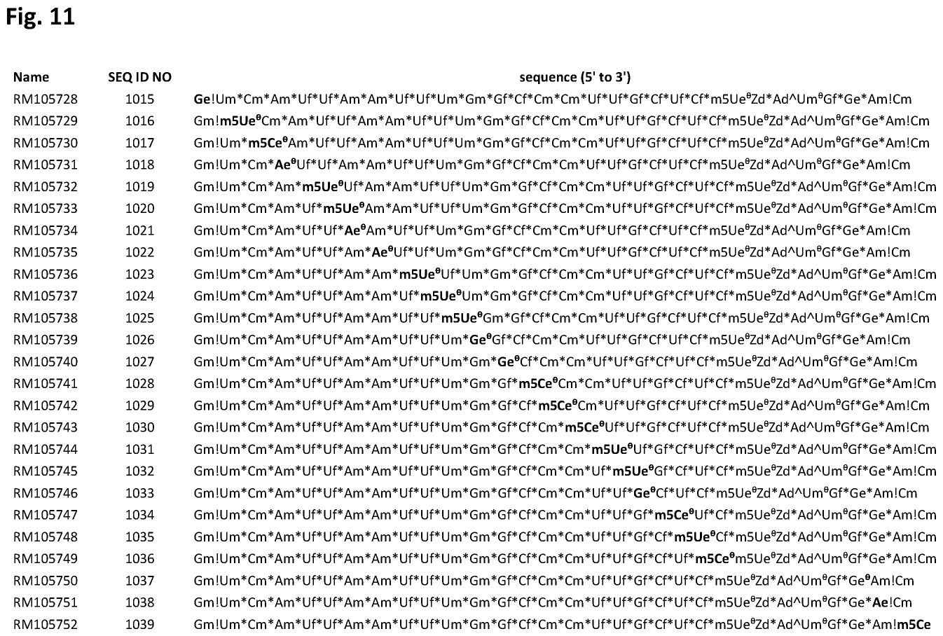

To identify whether introducing a 2′-MOE modification in combination with a 3′-located PO linkage would influence the editing efficiencies in deaminating the target adenosine in the codon for lysine at position 63, a set of EONs was designed, based on RM5059, in which at a variety of positions nucleotides and their 3′ located linkage was replaced by a nucleotide carrying a 2′-MOE substitution in the ribose and a PO linkage on the 3′ side. The designed EONs are provided in FIG. 11. The PNdmi linkages at the 5′ and 3′ termini were not replaced by PO. The orphan nucleotide (a deoxynucleotide carrying a Benner's base (Zd)) and the nucleotides at positions +1,-1,-2, and 3 were also not replaced. RM105733 and RM105752 were not manufactured and were not tested.

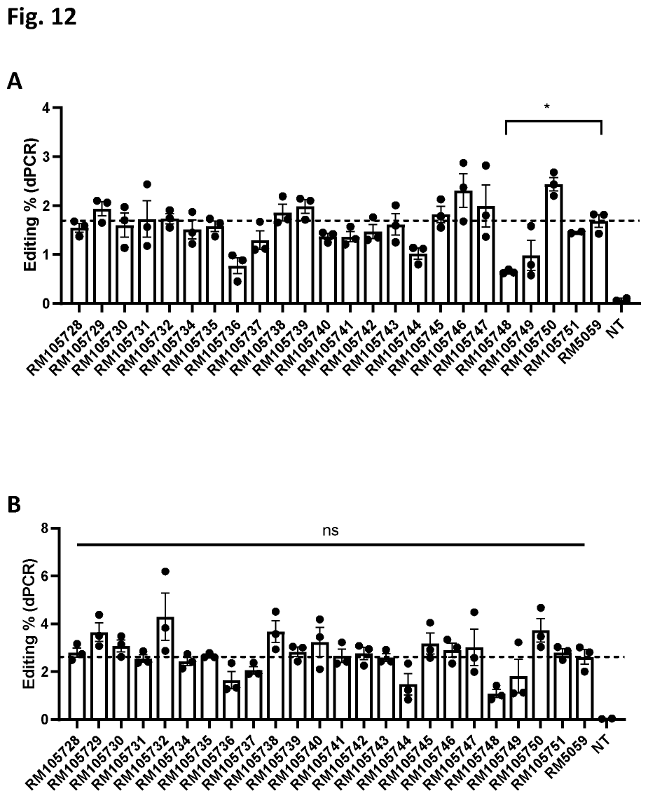

The EONs, together with the positive control EON RM5059 were introduced into PHH's using gymnotic uptake and using concentrations of 1 μM and 5 μM EON. Culturing, incubation, harvesting, RNA purification, cDNA generation and editing analysis was performed as described in example 1 and 2 above. The results of these experiments are shown in FIG. 12, in which the editing percentage of RM5059 was set as the standard (dotted line). Most EONs performed less than RM5059 except for RM105750, that—although not significantly—showed an increased editing rate. RM105750 comprises a PO linkage at linkage position −5, which is the only difference with RM5059. In contrast, RM105748 and RM105749 showed a decreased editing rate, with the decrease from RM5059 to RM105748 being significant. This suggests that PO linkages at linkage positions +1 and +2 are less desired, although it cannot be excluded that the presence of the 2′-MOE substitution in the ribose of nucleotide positions +2 and +3 also plays a role in this, albeit less likely. It is known in the art that PO linkages are prone to degradation by nucleases, which—in this case—may have caused a decrease in editing efficiency. From these experiments it can be concluded that introducing PO linkages at the 5′ side and close to the orphan nucleotide may influence the editing efficiency in a negative manner, while changing a PS linkage for a PO linkage at linkage position −5 may influence editing in a positive manner.

### Example 7. Editing of a Target Adenosine in a Human ANGPTL3 Target RNA Molecule in a High-Throughput Screen

Subsequently, a set of 960 EONs was designed (EON numbers RM114700 to RM115654, RM107905, RM115655 to RM115658; represented by SEQ ID NO:55 to 1014, respectively) comprising a variety of chemical and other modifications and all targeting the human ANGPTL3 transcript as discussed herein. These were tested as follows:

On day 0, PHHs (5.0×10⁴ cells/well) were transfected with EONs, in triplicates, using Lipofectamine® RNAiMAX Reagent at the same time of seeding, following the protocol of the manufacturer. The plates containing cells, medium and EON were held at 37° C., 5% CO₂ for 72 hrs, during which the medium was refreshed 24 hrs after transfection/plating.

On day 3 (72 hrs post transfection/plating) the supernatants were discarded, and subsequent analysis was performed as follows. Cells were collected and used for RNA isolation using a RNeasy 96 Kit (Qiagen-74182) according to the manufacturer's instructions. Extracted RNA was treated with DNase I (ThermoFisher-EN0521) according to manufacturer's protocol. Samples were incubated at 37° C. for 30 min and then 1 μL 50 mM EDTA was added and further incubated at 60° C. for 2 min. The total RNAs were then reverse-transcribed using the Maxima Reverse Transcriptase (Thermo-EP0742) kit with oligo-dT primer, random Hexamer Primer, and dNTP Mix (10 mM each). A quantitative PCR was then performed with the Digital PCR System (Bio-Rad, QX200) in 22 μl aliquots of reaction mixtures containing cDNA, appropriate pairs of primers and ddPCR Supermix for Probes (no dUTP) (Bio-Rad-1863024). The primers (SEQ ID NO: 32 to 38) given in Table 1 were used with a PCR program that was as follows: 10 min at 95° C.; 40 cycles for 30 sec at 94° C. and 60 sec at 63° C., 10 min at 98° C. and a hold step at 4° C. Then the plate was placed into the droplet QX200 reader to measure the number of positive droplets. The results were expressed as a percentage of mutant DNA alleles compared to total DNA alleles according to the formula:

target gene editing efficiency=target gene−G/(target gene−A+target gene−G)*100%

Triplicates were averaged and the final editing percentages were as given in Table 5 below, wherein the EONs are ranked from the highest editing percentage (averaged from three replicates) to the lowest. This shows that transfections with most EONs resulted in editing percentages below 1%. However, unexpectedly, some EONs performed significantly better, with RM107905, RM115657, RM115655, RM115656, RM115658, RM114862, RM115017, RM115225, RM115204, RM115151, and RM115179 performing best, with percentages above 10% and with some replicates going above 50%. It is noted that RM115504 (asterisk) contained an outlier of 60,00% in the third replicate.

| | repl- | repl- | repl- | Editing |
|---|---|---|---|---|
| EON | 1 | 2 | 3 | % |
| RM107905 | 48.31 | 49.22 | 48.51 | 48.68 |
| RM115657 | 47.55 | 51.09 | 47.20 | 48.61 |
| RM115655 | 47.20 | 49.92 | 47.48 | 48.20 |
| RM115656 | 41.57 | 44.19 | 42.07 | 42.61 |
| RM115658 | 22.29 | 21.05 | 20.66 | 21.33 |
| RM115504 | 0.88 | 1.38 | 60.00 | 20.75* |
| RM114862 | 21.89 | 17.42 | 17.99 | 19.10 |
| RM115017 | 16.85 | 14.93 | 13.45 | 15.08 |
| RM115225 | 11.10 | 17.61 | 11.37 | 13.36 |
| RM115204 | 11.42 | 13.25 | 11.76 | 12.15 |
| RM115151 | 10.64 | 10.92 | 11.36 | 10.97 |
| RM115179 | 10.08 | 10.14 | 11.20 | 10.47 |
| RM115057 | 11.63 | 8.48 | 9.65 | 9.92 |
| RM115412 | 9.33 | 10.38 | 9.04 | 9.58 |
| RM115240 | 7.92 | 12.46 | 8.10 | 9.49 |
| RM114834 | 9.40 | 8.06 | 10.63 | 9.37 |
| RM114999 | 8.58 | 8.58 | 9.85 | 9.00 |
| RM115110 | 8.60 | 7.75 | 10.12 | 8.82 |
| RM114751 | 11.49 | 6.67 | 8.31 | 8.82 |
| RM115180 | 7.98 | 8.20 | 9.19 | 8.45 |
| RM115322 | 8.67 | 7.55 | 8.98 | 8.40 |
| RM114829 | 6.89 | 6.05 | 8.90 | 7.28 |
| RM115272 | 7.61 | 7.99 | 5.84 | 7.15 |
| RM114740 | 5.94 | 7.20 | 7.58 | 6.90 |
| RM115008 | 6.58 | 7.98 | 5.94 | 6.83 |
| RM115450 | 7.06 | 6.80 | 5.30 | 6.39 |
| RM115602 | 5.65 | 7.45 | 6.04 | 6.38 |
| RM114924 | 6.58 | 5.75 | 6.13 | 6.15 |
| RM114922 | 7.39 | 5.52 | 5.02 | 5.97 |
| RM115012 | 5.43 | 5.61 | 5.95 | 5.67 |
| RM114741 | 5.69 | 5.92 | 5.08 | 5.56 |
| RM115168 | 4.58 | 5.85 | 5.71 | 5.38 |
| RM115601 | 5.01 | 5.57 | 5.18 | 5.25 |
| RM115024 | 5.25 | 4.63 | 5.10 | 4.99 |
| RM115323 | 6.02 | 4.97 | 3.93 | 4.97 |
| RM115605 | 3.95 | 6.42 | 4.55 | 4.97 |
| RM114801 | 4.80 | 4.90 | 5.17 | 4.96 |
| RM114869 | 5.31 | 4.85 | 4.69 | 4.95 |
| RM115606 | 4.73 | 4.44 | 5.36 | 4.85 |
| RM114847 | 4.18 | 4.69 | 4.26 | 4.38 |
| RM115471 | 4.15 | 3.27 | 5.54 | 4.32 |
| RM114888 | 3.41 | 4.43 | 4.80 | 4.21 |
| RM115141 | 4.01 | 4.64 | 3.82 | 4.16 |
| RM115070 | 3.53 | 5.12 | 3.56 | 4.07 |
| RM115171 | 3.83 | 4.60 | 3.77 | 4.07 |
| RM114955 | 3.64 | 4.21 | 4.15 | 4.00 |
| RM114752 | 3.63 | 4.13 | 3.98 | 3.91 |
| RM114945 | 3.84 | 3.81 | 3.76 | 3.80 |
| RM115325 | 3.42 | 3.29 | 4.26 | 3.66 |
| RM115434 | 3.76 | 3.94 | 3.09 | 3.60 |
| RM115183 | 3.96 | 3.80 | 3.00 | 3.59 |
| RM115413 | 2.84 | 4.03 | 3.66 | 3.51 |
| RM114968 | 3.55 | 3.07 | 3.64 | 3.42 |
| RM114744 | 3.27 | 3.47 | 3.48 | 3.41 |
| RM115195 | 3.90 | 3.05 | 3.20 | 3.38 |
| RM114983 | 3.41 | 3.33 | 3.29 | 3.34 |
| RM115607 | 2.61 | 2.42 | 4.85 | 3.29 |
| RM115281 | 3.02 | 2.64 | 3.98 | 3.21 |
| RM115611 | 2.79 | 3.42 | 3.20 | 3.14 |
| RM114880 | 3.26 | 2.70 | 3.05 | 3.00 |
| RM114975 | 3.21 | 2.91 | 2.88 | 3.00 |
| RM115419 | 2.66 | 3.64 | 2.44 | 2.91 |
| RM114802 | 3.13 | 2.10 | 3.10 | 2.78 |
| RM115289 | 2.76 | 2.24 | 3.24 | 2.75 |
| RM115152 | 2.65 | 2.88 | 2.61 | 2.71 |
| RM114993 | 2.78 | 2.38 | 2.84 | 2.66 |
| RM114895 | 2.61 | 2.26 | 2.78 | 2.55 |
| RM114988 | 2.47 | 2.39 | 2.68 | 2.51 |
| RM115145 | 1.73 | 3.61 | 2.18 | 2.51 |
| RM115654 | 1.43 | 1.02 | 1.37 | 1.27 |
| RM114889 | 1.32 | 1.35 | 1.13 | 1.27 |
| RM114843 | 0.97 | 1.51 | 1.29 | 1.26 |
| RM115065 | 1.43 | 1.06 | 1.25 | 1.25 |
| RM114727 | 1.13 | 1.40 | 1.15 | 1.22 |
| RM114995 | 0.96 | 1.11 | 1.59 | 1.22 |
| RM115431 | 1.10 | 1.37 | 1.18 | 1.22 |
| RM114798 | 1.23 | 1.06 | 1.36 | 1.22 |
| RM115290 | 0.93 | 0.99 | 1.69 | 1.20 |
| RM115090 | 1.41 | 0.51 | 1.69 | 1.20 |
| RM115003 | 1.23 | 1.14 | 1.22 | 1.20 |
| RM115146 | 1.48 | 1.02 | 1.08 | 1.19 |
| RM115071 | 1.11 | 1.26 | 1.18 | 1.18 |
| RM115258 | 1.08 | 1.31 | 1.16 | 1.18 |
| RM114996 | 1.23 | 0.87 | 1.41 | 1.17 |
| RM114851 | 1.39 | 1.14 | 0.97 | 1.17 |
| RM115559 | 0.72 | 1.36 | 1.42 | 1.17 |
| RM115326 | 1.15 | 1.37 | 0.93 | 1.15 |
| RM115597 | 1.04 | 1.14 | 1.27 | 1.15 |
| RM115393 | 1.40 | 1.04 | 1.00 | 1.15 |
| RM115212 | 1.06 | 1.04 | 1.33 | 1.14 |
| RM115315 | 0.87 | 1.48 | 1.08 | 1.14 |
| RM114896 | 1.06 | 1.10 | 1.27 | 1.14 |
| RM115038 | 1.16 | 1.11 | 1.14 | 1.14 |
| RM115517 | 1.40 | 1.19 | 0.80 | 1.13 |
| RM114742 | 1.17 | 1.17 | 1.04 | 1.12 |
| RM115020 | 1.26 | 1.26 | 0.85 | 1.12 |
| RM115332 | 1.32 | 0.60 | 1.37 | 1.10 |
| RM115199 | 1.09 | 0.94 | 1.27 | 1.10 |
| RM115634 | 1.51 | 1.23 | 0.55 | 1.10 |
| RM115282 | 1.74 | 0.62 | 0.93 | 1.10 |
| RM114870 | 1.03 | 1.11 | 1.11 | 1.08 |
| RM114747 | 1.03 | 0.87 | 1.33 | 1.08 |
| RM115463 | 1.14 | 1.08 | 0.99 | 1.07 |
| RM114777 | 1.24 | 0.53 | 1.44 | 1.07 |
| RM115050 | 1.25 | 1.12 | 0.82 | 1.06 |
| RM115163 | 0.99 | 1.42 | 0.78 | 1.06 |
| RM114859 | 0.98 | 0.85 | 1.36 | 1.06 |
| RM114731 | 1.17 | 1.29 | 0.71 | 1.06 |
| RM115335 | 0.97 | 1.11 | 1.09 | 1.06 |
| RM115535 | 0.93 | 1.02 | 1.18 | 1.04 |
| RM115651 | 1.05 | 0.87 | 1.19 | 1.04 |
| RM115018 | 1.10 | 1.01 | 1.00 | 1.04 |
| RM115428 | 0.90 | 1.26 | 0.92 | 1.03 |
| RM114956 | 1.07 | 1.19 | 0.82 | 1.03 |
| RM114756 | 0.71 | 1.25 | 1.12 | 1.03 |
| RM114792 | 1.27 | 0.82 | 0.98 | 1.03 |
| RM114748 | 1.29 | 1.18 | 0.61 | 1.02 |
| RM115250 | 0.90 | 1.28 | 0.87 | 1.02 |
| RM115615 | 0.89 | 1.10 | 1.02 | 1.00 |
| RM115552 | 0.39 | 0.93 | 1.69 | 1.00 |
| RM114723 | 1.14 | 0.71 | 1.14 | 1.00 |
| RM115586 | 1.03 | 1.11 | 0.84 | 1.00 |
| RM115467 | 0.70 | 1.22 | 1.06 | 0.99 |
| RM115172 | 1.27 | 1.26 | 0.44 | 0.99 |
| RM115579 | 0.95 | 0.83 | 1.19 | 0.99 |
| RM114755 | 1.02 | 1.02 | 0.93 | 0.99 |
| RM115155 | 0.91 | 1.20 | 0.84 | 0.99 |
| RM115441 | 0.74 | 0.57 | 1.63 | 0.98 |
| RM115619 | 1.02 | 0.91 | 0.97 | 0.96 |
| RM115540 | 0.79 | 0.87 | 1.22 | 0.96 |
| RM115324 | 0.89 | 1.12 | 0.86 | 0.96 |
| RM114986 | 0.85 | 1.08 | 0.88 | 0.94 |
| RM115232 | 0.75 | 1.00 | 1.05 | 0.93 |
| RM114838 | 0.92 | 1.18 | 0.70 | 0.93 |
| RM114738 | 0.98 | 0.76 | 1.06 | 0.93 |
| RM115136 | 1.01 | 0.78 | 0.95 | 0.91 |
| RM115229 | 0.82 | 1.19 | 0.70 | 0.90 |
| RM115269 | 1.05 | 0.55 | 1.11 | 0.90 |
| RM115001 | 0.99 | 0.45 | 0.67 | 0.70 |
| RM114899 | 0.68 | 0.65 | 0.77 | 0.70 |
| RM115404 | 0.66 | 0.67 | 0.77 | 0.70 |
| RM115081 | 0.87 | 0.42 | 0.81 | 0.70 |
| RM115609 | 0.62 | 0.65 | 0.80 | 0.69 |
| RM115342 | 0.63 | 0.91 | 0.53 | 0.69 |
| RM115409 | 0.93 | 0.54 | 0.58 | 0.68 |
| RM115461 | 0.44 | 0.77 | 0.83 | 0.68 |
| RM115085 | 0.91 | 0.29 | 0.84 | 0.68 |
| RM115074 | 0.61 | 0.74 | 0.68 | 0.68 |
| RM114932 | 0.81 | 0.56 | 0.66 | 0.68 |
| RM115137 | 0.80 | 0.62 | 0.61 | 0.68 |
| RM115593 | 0.75 | 0.67 | 0.59 | 0.67 |
| RM114728 | 0.83 | 0.69 | 0.48 | 0.67 |
| RM114850 | 0.74 | 0.43 | 0.84 | 0.67 |
| RM115603 | 0.53 | 0.70 | 0.78 | 0.67 |
| RM115051 | 0.83 | 0.27 | 0.91 | 0.67 |
| RM114767 | 0.82 | 0.41 | 0.76 | 0.67 |
| RM114753 | 0.74 | 0.55 | 0.70 | 0.67 |
| RM114791 | 0.53 | 0.35 | 1.12 | 0.66 |
| RM115207 | 0.69 | 0.81 | 0.49 | 0.66 |
| RM115458 | 0.55 | 0.86 | 0.56 | 0.66 |
| RM115314 | 0.98 | 0.83 | 0.16 | 0.66 |
| RM115072 | 0.43 | 0.89 | 0.65 | 0.66 |
| RM115045 | 0.48 | 0.93 | 0.56 | 0.65 |
| RM115495 | 0.75 | 0.56 | 0.65 | 0.65 |
| RM115153 | 0.46 | 0.79 | 0.71 | 0.65 |
| RM115293 | 0.75 | 0.58 | 0.61 | 0.65 |
| RM115414 | 0.70 | 0.40 | 0.84 | 0.65 |
| RM115632 | 0.69 | 0.63 | 0.62 | 0.65 |
| RM115211 | 0.64 | 0.62 | 0.68 | 0.64 |
| RM115084 | 0.70 | 0.74 | 0.49 | 0.64 |
| RM115440 | 0.62 | 0.69 | 0.60 | 0.64 |
| RM115192 | 0.69 | 0.72 | 0.51 | 0.64 |
| RM114733 | 0.77 | 0.81 | 0.33 | 0.64 |
| RM115088 | 0.84 | 0.58 | 0.49 | 0.63 |
| RM115416 | 0.66 | 0.24 | 0.99 | 0.63 |
| RM114890 | 0.65 | 0.71 | 0.53 | 0.63 |
| RM114856 | 0.44 | 0.73 | 0.72 | 0.63 |
| RM115555 | 0.56 | 0.76 | 0.57 | 0.63 |
| RM115343 | 0.70 | 0.51 | 0.67 | 0.63 |
| RM115177 | 0.49 | 0.65 | 0.74 | 0.63 |
| RM115351 | 0.41 | 0.85 | 0.61 | 0.63 |
| RM115411 | 0.49 | 0.45 | 0.93 | 0.62 |
| RM115318 | 0.49 | 0.74 | 0.64 | 0.62 |
| RM115356 | 0.65 | 0.63 | 0.59 | 0.62 |
| RM114916 | 0.66 | 0.51 | 0.69 | 0.62 |
| RM115403 | 0.63 | 0.66 | 0.58 | 0.62 |
| RM115242 | 0.59 | 0.75 | 0.52 | 0.62 |
| RM115200 | 0.54 | 0.68 | 0.64 | 0.62 |
| RM114874 | 0.38 | 0.73 | 0.75 | 0.62 |
| RM115330 | 0.57 | 0.49 | 0.78 | 0.62 |
| RM115127 | 0.62 | 0.69 | 0.54 | 0.61 |
| RM115245 | 0.56 | 0.52 | 0.75 | 0.61 |
| RM114979 | 0.58 | 0.75 | 0.48 | 0.60 |
| RM115243 | 0.76 | 0.53 | 0.52 | 0.60 |
| RM115362 | 0.51 | 0.53 | 0.77 | 0.60 |
| RM115349 | 0.68 | 0.69 | 0.43 | 0.60 |
| RM115473 | 0.68 | 0.48 | 0.64 | 0.60 |
| RM115447 | 0.84 | 0.59 | 0.37 | 0.60 |
| RM114910 | 0.40 | 0.65 | 0.74 | 0.60 |
| RM114842 | 0.59 | 0.67 | 0.53 | 0.60 |
| RM115055 | 0.56 | 0.56 | 0.67 | 0.60 |
| RM114769 | 0.67 | 0.57 | 0.55 | 0.60 |
| RM115613 | 0.60 | 0.47 | 0.72 | 0.59 |
| RM115224 | 0.96 | 0.41 | 0.40 | 0.59 |
| RM115406 | 0.75 | 0.58 | 0.45 | 0.59 |
| RM115213 | 0.61 | 0.50 | 0.67 | 0.59 |
| RM115203 | 0.60 | 0.73 | 0.46 | 0.59 |
| RM115156 | 0.49 | 0.53 | 0.50 | 0.51 |
| RM115438 | 0.49 | 0.46 | 0.57 | 0.50 |
| RM114972 | 0.66 | 0.45 | 0.39 | 0.50 |
| RM115333 | 0.41 | 0.71 | 0.38 | 0.50 |
| RM115022 | 0.57 | 0.41 | 0.52 | 0.50 |
| RM114790 | 0.51 | 0.62 | 0.37 | 0.50 |
| RM115444 | 0.51 | 0.49 | 0.49 | 0.50 |
| RM115123 | 0.50 | 0.43 | 0.56 | 0.50 |
| RM114735 | 0.35 | 0.66 | 0.48 | 0.50 |
| RM115347 | 0.33 | 0.64 | 0.51 | 0.50 |
| RM115373 | 0.54 | 0.47 | 0.48 | 0.50 |
| RM114771 | 0.37 | 0.68 | 0.43 | 0.49 |
| RM115265 | 0.45 | 0.57 | 0.46 | 0.49 |
| RM115307 | 0.33 | 0.68 | 0.47 | 0.49 |
| RM115099 | 0.46 | 0.65 | 0.36 | 0.49 |
| RM115625 | 0.43 | 0.37 | 0.68 | 0.49 |
| RM114818 | 0.59 | 0.41 | 0.46 | 0.49 |
| RM114739 | 0.58 | 0.52 | 0.37 | 0.49 |
| RM115154 | 0.45 | 0.57 | 0.44 | 0.49 |
| RM115442 | 0.48 | 0.50 | 0.47 | 0.49 |
| RM115381 | 0.19 | 0.51 | 0.77 | 0.49 |
| RM115500 | 0.44 | 0.45 | 0.57 | 0.49 |
| RM115283 | 0.57 | 0.45 | 0.44 | 0.48 |
| RM114978 | 0.69 | 0.47 | 0.29 | 0.48 |
| RM115317 | 0.57 | 0.39 | 0.49 | 0.48 |
| RM115528 | 0.31 | 0.68 | 0.46 | 0.48 |
| RM115262 | 0.56 | 0.43 | 0.45 | 0.48 |
| RM115485 | 0.68 | 0.63 | 0.14 | 0.48 |
| RM115286 | 0.44 | 0.55 | 0.46 | 0.48 |
| RM115062 | 0.66 | 0.42 | 0.36 | 0.48 |
| RM115034 | 0.43 | 0.37 | 0.63 | 0.48 |
| RM115275 | 0.45 | 0.45 | 0.53 | 0.48 |
| RM115264 | 0.21 | 0.34 | 0.87 | 0.47 |
| RM114991 | 0.50 | 0.47 | 0.45 | 0.47 |
| RM115631 | 0.46 | 0.55 | 0.41 | 0.47 |
| RM115076 | 0.35 | 0.70 | 0.37 | 0.47 |
| RM115427 | 0.49 | 0.53 | 0.40 | 0.47 |
| RM115592 | 0.60 | 0.37 | 0.43 | 0.47 |
| RM115279 | 0.34 | 0.42 | 0.64 | 0.47 |
| RM115430 | 0.39 | 0.53 | 0.49 | 0.47 |
| RM115377 | 0.48 | 0.53 | 0.40 | 0.47 |
| RM114763 | 0.34 | 0.54 | 0.52 | 0.47 |
| RM115513 | 0.22 | 0.60 | 0.58 | 0.47 |
| RM114893 | 0.61 | 0.41 | 0.37 | 0.47 |
| RM114902 | 0.56 | 0.49 | 0.35 | 0.46 |
| RM115635 | 0.49 | 0.42 | 0.48 | 0.46 |
| RM115544 | 0.38 | 0.46 | 0.55 | 0.46 |
| RM114950 | 0.40 | 0.52 | 0.46 | 0.46 |
| RM114864 | 0.50 | 0.49 | 0.39 | 0.46 |
| RM115423 | 0.38 | 0.35 | 0.64 | 0.46 |
| RM115316 | 0.41 | 0.31 | 0.66 | 0.46 |
| RM115310 | 0.48 | 0.49 | 0.39 | 0.46 |
| RM114781 | 0.57 | 0.21 | 0.58 | 0.46 |
| RM115027 | 0.51 | 0.37 | 0.49 | 0.45 |
| RM115379 | 0.61 | 0.30 | 0.45 | 0.45 |
| RM115474 | 0.41 | 0.38 | 0.57 | 0.45 |
| RM115548 | 0.48 | 0.47 | 0.40 | 0.45 |
| RM115339 | 0.32 | 0.54 | 0.48 | 0.45 |
| RM115236 | 0.41 | 0.51 | 0.42 | 0.45 |
| RM115571 | 0.29 | 0.39 | 0.66 | 0.45 |
| RM114891 | 0.24 | 0.69 | 0.41 | 0.45 |
| RM115456 | 0.55 | 0.40 | 0.37 | 0.44 |
| RM114957 | 0.36 | 0.52 | 0.43 | 0.44 |
| RM115233 | 0.52 | 0.31 | 0.48 | 0.44 |
| RM115394 | 0.58 | 0.53 | 0.19 | 0.44 |
| RM115616 | 0.52 | 0.40 | 0.39 | 0.44 |
| RM115047 | 0.60 | 0.27 | 0.43 | 0.43 |
| RM115645 | 0.43 | 0.42 | 0.44 | 0.43 |
| RM115073 | 0.35 | 0.75 | 0.20 | 0.43 |
| RM115389 | 0.38 | 0.42 | 0.23 | 0.34 |
| RM115004 | 0.32 | 0.34 | 0.36 | 0.34 |
| RM115525 | 0.44 | 0.33 | 0.24 | 0.34 |
| RM114776 | 0.35 | 0.42 | 0.24 | 0.34 |
| RM114715 | 0.32 | 0.46 | 0.22 | 0.34 |
| RM115303 | 0.30 | 0.26 | 0.44 | 0.33 |
| RM115496 | 0.58 | 0.04 | 0.38 | 0.33 |
| RM115297 | 0.50 | 0.20 | 0.30 | 0.33 |
| RM114822 | 0.36 | 0.46 | 0.18 | 0.33 |
| RM115365 | 0.19 | 0.08 | 0.73 | 0.33 |
| RM115010 | 0.49 | 0.25 | 0.25 | 0.33 |
| RM114941 | 0.34 | 0.19 | 0.47 | 0.33 |
| RM115125 | 0.37 | 0.25 | 0.37 | 0.33 |
| RM115486 | 0.30 | 0.29 | 0.40 | 0.33 |
| RM115375 | 0.29 | 0.41 | 0.29 | 0.33 |
| RM115014 | 0.34 | 0.24 | 0.42 | 0.33 |
| RM115078 | 0.24 | 0.42 | 0.32 | 0.33 |
| RM115220 | 0.31 | 0.35 | 0.32 | 0.33 |
| RM115299 | 0.25 | 0.27 | 0.47 | 0.33 |
| RM115328 | 0.53 | 0.18 | 0.27 | 0.33 |
| RM115492 | 0.46 | 0.35 | 0.17 | 0.33 |
| RM115591 | 0.46 | 0.21 | 0.31 | 0.33 |
| RM114721 | 0.00 | 0.57 | 0.41 | 0.33 |
| RM114970 | 0.49 | 0.12 | 0.37 | 0.33 |
| RM115312 | 0.20 | 0.47 | 0.31 | 0.32 |
| RM115105 | 0.12 | 0.49 | 0.36 | 0.32 |
| RM114794 | 0.28 | 0.36 | 0.33 | 0.32 |
| RM115109 | 0.67 | 0.09 | 0.21 | 0.32 |
| RM114788 | 0.31 | 0.51 | 0.14 | 0.32 |
| RM114749 | 0.25 | 0.33 | 0.38 | 0.32 |
| RM115432 | 0.41 | 0.24 | 0.31 | 0.32 |
| RM115223 | 0.27 | 0.43 | 0.26 | 0.32 |
| RM115133 | 0.38 | 0.29 | 0.29 | 0.32 |
| RM115580 | 0.26 | 0.41 | 0.29 | 0.32 |
| RM115518 | 0.31 | 0.41 | 0.23 | 0.32 |
| RM115541 | 0.36 | 0.38 | 0.22 | 0.32 |
| RM115260 | 0.38 | 0.27 | 0.31 | 0.32 |
| RM115424 | 0.33 | 0.35 | 0.28 | 0.32 |
| RM115288 | 0.21 | 0.08 | 0.66 | 0.32 |
| RM115514 | 0.29 | 0.40 | 0.26 | 0.32 |
| RM114724 | 0.33 | 0.40 | 0.21 | 0.31 |
| RM114754 | 0.59 | 0.18 | 0.17 | 0.31 |
| RM115624 | 0.11 | 0.58 | 0.25 | 0.31 |
| RM115498 | 0.31 | 0.38 | 0.26 | 0.31 |
| RM115148 | 0.18 | 0.51 | 0.25 | 0.31 |
| RM115476 | 0.27 | 0.39 | 0.27 | 0.31 |
| RM115015 | 0.40 | 0.25 | 0.28 | 0.31 |
| RM115327 | 0.27 | 0.46 | 0.20 | 0.31 |
| RM115422 | 0.25 | 0.27 | 0.42 | 0.31 |
| RM115202 | 0.41 | 0.23 | 0.29 | 0.31 |
| RM114857 | 0.33 | 0.21 | 0.38 | 0.31 |
| RM114840 | 0.31 | 0.27 | 0.34 | 0.31 |
| RM114711 | 0.26 | 0.40 | 0.27 | 0.31 |
| RM115311 | 0.42 | 0.19 | 0.31 | 0.31 |
| RM115007 | 0.30 | 0.29 | 0.33 | 0.31 |
| RM114701 | 0.38 | 0.24 | 0.29 | 0.30 |
| RM115537 | 0.24 | 0.27 | 0.40 | 0.30 |
| RM114947 | 0.27 | 0.35 | 0.28 | 0.30 |
| RM115252 | 0.44 | 0.29 | 0.17 | 0.30 |
| RM115271 | 0.47 | 0.22 | 0.21 | 0.30 |
| RM115519 | 0.36 | 0.23 | 0.30 | 0.30 |
| RM114764 | 0.25 | 0.33 | 0.32 | 0.30 |
| RM115575 | 0.35 | 0.19 | 0.36 | 0.30 |
| RM114944 | 0.28 | 0.21 | 0.40 | 0.30 |
| RM114706 | 0.27 | 0.32 | 0.30 | 0.30 |
| RM115497 | 0.20 | 0.30 | 0.38 | 0.30 |
| RM114819 | 0.26 | 0.35 | 0.28 | 0.30 |
| RM115459 | 0.37 | 0.19 | 0.32 | 0.29 |
| RM115556 | 0.28 | 0.30 | 0.31 | 0.29 |
| RM115360 | 0.17 | 0.21 | 0.30 | 0.23 |
| RM115111 | 0.28 | 0.18 | 0.22 | 0.23 |
| RM115226 | 0.18 | 0.19 | 0.31 | 0.23 |
| RM115577 | 0.16 | 0.10 | 0.42 | 0.23 |
| RM115652 | 0.29 | 0.19 | 0.19 | 0.23 |
| RM114905 | 0.18 | 0.20 | 0.29 | 0.23 |
| RM115132 | 0.25 | 0.21 | 0.21 | 0.22 |
| RM115083 | 0.27 | 0.20 | 0.20 | 0.22 |
| RM115348 | 0.40 | 0.10 | 0.17 | 0.22 |
| RM114809 | 0.24 | 0.05 | 0.38 | 0.22 |
| RM115135 | 0.15 | 0.23 | 0.28 | 0.22 |
| RM115421 | 0.40 | 0.06 | 0.21 | 0.22 |
| RM114868 | 0.20 | 0.36 | 0.09 | 0.22 |
| RM115526 | 0.22 | 0.22 | 0.22 | 0.22 |
| RM115520 | 0.24 | 0.20 | 0.21 | 0.22 |
| RM115452 | 0.14 | 0.20 | 0.31 | 0.22 |
| RM115569 | 0.16 | 0.20 | 0.29 | 0.21 |
| RM114812 | 0.26 | 0.18 | 0.20 | 0.21 |
| RM115531 | 0.13 | 0.25 | 0.25 | 0.21 |
| RM115066 | 0.22 | 0.20 | 0.22 | 0.21 |
| RM115582 | 0.20 | 0.15 | 0.28 | 0.21 |
| RM114919 | 0.00 | 0.21 | 0.42 | 0.21 |
| RM115558 | 0.21 | 0.14 | 0.29 | 0.21 |
| RM115402 | 0.24 | 0.20 | 0.19 | 0.21 |
| RM115308 | 0.12 | 0.17 | 0.34 | 0.21 |
| RM115159 | 0.21 | 0.20 | 0.21 | 0.21 |
| RM114806 | 0.27 | 0.15 | 0.20 | 0.21 |
| RM114702 | 0.21 | 0.17 | 0.24 | 0.21 |
| RM115554 | 0.09 | 0.32 | 0.20 | 0.21 |
| RM114807 | 0.34 | 0.14 | 0.14 | 0.20 |
| RM115491 | 0.21 | 0.16 | 0.24 | 0.20 |
| RM115358 | 0.16 | 0.44 | 0.00 | 0.20 |
| RM115185 | 0.21 | 0.16 | 0.23 | 0.20 |
| RM115251 | 0.06 | 0.36 | 0.18 | 0.20 |
| RM115209 | 0.14 | 0.24 | 0.22 | 0.20 |
| RM114936 | 0.13 | 0.24 | 0.23 | 0.20 |
| RM115588 | 0.27 | 0.20 | 0.13 | 0.20 |
| RM115439 | 0.19 | 0.19 | 0.21 | 0.20 |
| RM114861 | 0.14 | 0.22 | 0.23 | 0.20 |
| RM115385 | 0.19 | 0.15 | 0.24 | 0.20 |
| RM115140 | 0.04 | 0.26 | 0.28 | 0.19 |
| RM115093 | 0.22 | 0.28 | 0.08 | 0.19 |
| RM115178 | 0.15 | 0.18 | 0.24 | 0.19 |
| RM115113 | 0.20 | 0.22 | 0.16 | 0.19 |
| RM115536 | 0.30 | 0.09 | 0.18 | 0.19 |
| RM115119 | 0.25 | 0.15 | 0.17 | 0.19 |
| RM115340 | 0.18 | 0.24 | 0.14 | 0.19 |
| RM114954 | 0.17 | 0.29 | 0.11 | 0.19 |
| RM115567 | 0.20 | 0.18 | 0.19 | 0.19 |
| RM115030 | 0.22 | 0.15 | 0.19 | 0.19 |
| RM114805 | 0.09 | 0.32 | 0.15 | 0.19 |
| RM115086 | 0.18 | 0.20 | 0.18 | 0.19 |
| RM115493 | 0.15 | 0.18 | 0.23 | 0.19 |
| RM115352 | 0.17 | 0.19 | 0.19 | 0.19 |
| RM115194 | 0.25 | 0.13 | 0.18 | 0.18 |
| RM114782 | 0.12 | 0.17 | 0.26 | 0.18 |
| RM115253 | 0.16 | 0.11 | 0.28 | 0.18 |
| RM115533 | 0.38 | 0.17 | 0.00 | 0.18 |
| RM114827 | 0.00 | 0.22 | 0.32 | 0.18 |
| RM115516 | 0.19 | 0.19 | 0.16 | 0.18 |
| RM115149 | 0.18 | 0.27 | 0.09 | 0.18 |
| RM115647 | 0.11 | 0.26 | 0.16 | 0.18 |
| RM115296 | 0.17 | 0.15 | 0.21 | 0.18 |
| RM115005 | 0.22 | 0.21 | 0.10 | 0.18 |
| RM115633 | 0.11 | 0.22 | 0.18 | 0.17 |
| RM115304 | 0.21 | 0.17 | 0.14 | 0.17 |
| RM115512 | 0.14 | 0.23 | 0.15 | 0.17 |
| RM114814 | 0.13 | 0.27 | 0.12 | 0.17 |
| RM115023 | 0.07 | 0.22 | 0.22 | 0.17 |
| RM115158 | 0.12 | 0.12 | 0.14 | 0.13 |
| RM114774 | 0.08 | 0.17 | 0.13 | 0.13 |
| RM114967 | 0.10 | 0.05 | 0.23 | 0.13 |
| RM115227 | 0.13 | 0.13 | 0.11 | 0.13 |
| RM114732 | 0.14 | 0.10 | 0.13 | 0.13 |
| RM114990 | 0.08 | 0.29 | 0.00 | 0.12 |
| RM115503 | 0.13 | 0.09 | 0.16 | 0.12 |
| RM115261 | 0.12 | 0.14 | 0.11 | 0.12 |
| RM115564 | 0.11 | 0.12 | 0.13 | 0.12 |
| RM114914 | 0.11 | 0.09 | 0.16 | 0.12 |
| RM115641 | 0.13 | 0.17 | 0.07 | 0.12 |
| RM115581 | 0.09 | 0.22 | 0.06 | 0.12 |
| RM115019 | 0.21 | 0.00 | 0.15 | 0.12 |
| RM115477 | 0.12 | 0.12 | 0.13 | 0.12 |
| RM114897 | 0.26 | 0.11 | 0.00 | 0.12 |
| RM115638 | 0.11 | 0.22 | 0.03 | 0.12 |
| RM115549 | 0.21 | 0.05 | 0.11 | 0.12 |
| RM115573 | 0.23 | 0.13 | 0.00 | 0.12 |
| RM115301 | 0.19 | 0.08 | 0.09 | 0.12 |
| RM115470 | 0.25 | 0.00 | 0.11 | 0.12 |
| RM115234 | 0.09 | 0.10 | 0.15 | 0.12 |
| RM115331 | 0.18 | 0.09 | 0.08 | 0.12 |
| RM115578 | 0.15 | 0.12 | 0.08 | 0.12 |
| RM114810 | 0.11 | 0.06 | 0.18 | 0.12 |
| RM115621 | 0.02 | 0.22 | 0.10 | 0.12 |
| RM115031 | 0.07 | 0.16 | 0.11 | 0.12 |
| RM114785 | 0.00 | 0.19 | 0.16 | 0.12 |
| RM114717 | 0.18 | 0.08 | 0.09 | 0.11 |
| RM115387 | 0.15 | 0.03 | 0.16 | 0.11 |
| RM115366 | 0.12 | 0.05 | 0.17 | 0.11 |
| RM115101 | 0.16 | 0.00 | 0.18 | 0.11 |
| RM115256 | 0.07 | 0.17 | 0.10 | 0.11 |
| RM115515 | 0.18 | 0.10 | 0.06 | 0.11 |
| RM115464 | 0.16 | 0.08 | 0.10 | 0.11 |
| RM115107 | 0.07 | 0.10 | 0.16 | 0.11 |
| RM114933 | 0.19 | 0.10 | 0.04 | 0.11 |
| RM114985 | 0.14 | 0.10 | 0.09 | 0.11 |
| RM115401 | 0.13 | 0.09 | 0.11 | 0.11 |
| RM115561 | 0.16 | 0.08 | 0.10 | 0.11 |
| RM115214 | 0.15 | 0.08 | 0.10 | 0.11 |
| RM114960 | 0.12 | 0.12 | 0.08 | 0.11 |
| RM115193 | 0.14 | 0.09 | 0.10 | 0.11 |
| RM115643 | 0.12 | 0.11 | 0.10 | 0.11 |
| RM114928 | 0.08 | 0.09 | 0.15 | 0.11 |
| RM115397 | 0.09 | 0.12 | 0.10 | 0.11 |
| RM114855 | 0.19 | 0.04 | 0.09 | 0.11 |
| RM115639 | 0.07 | 0.08 | 0.17 | 0.11 |
| RM114903 | 0.10 | 0.14 | 0.08 | 0.10 |
| RM115287 | 0.14 | 0.07 | 0.10 | 0.10 |
| RM114783 | 0.08 | 0.14 | 0.08 | 0.10 |
| RM115092 | 0.09 | 0.07 | 0.15 | 0.10 |
| RM114846 | 0.10 | 0.06 | 0.15 | 0.10 |
| RM115218 | 0.03 | 0.14 | 0.14 | 0.10 |
| RM115382 | 0.15 | 0.10 | 0.05 | 0.10 |
| RM114961 | 0.13 | 0.08 | 0.10 | 0.10 |
| RM115502 | 0.04 | 0.10 | 0.16 | 0.10 |
| RM115210 | 0.12 | 0.09 | 0.09 | 0.10 |
| RM115187 | 0.07 | 0.15 | 0.08 | 0.10 |
| RM115247 | 0.11 | 0.09 | 0.10 | 0.10 |
| RM115079 | 0.11 | 0.05 | 0.14 | 0.10 |
| RM115046 | 0.03 | 0.06 | 0.20 | 0.10 |
| RM114937 | 0.16 | 0.04 | 0.10 | 0.10 |
| RM114797 | 0.06 | 0.09 | 0.15 | 0.10 |
| RM115369 | 0.00 | 0.18 | 0.11 | 0.10 |
| RM114704 | 0.09 | 0.13 | 0.07 | 0.10 |
| RM115368 | 0.12 | 0.08 | 0.10 | 0.10 |
| RM115056 | 0.15 | 0.06 | 0.08 | 0.10 |
| RM114853 | 0.08 | 0.17 | 0.03 | 0.10 |
| RM115341 | 0.10 | 0.09 | 0.10 | 0.10 |
| RM115205 | 2.91 | 2.38 | 2.18 | 2.49 |
| RM115144 | 2.15 | 3.02 | 2.29 | 2.49 |
| RM115241 | 2.06 | 2.83 | 2.37 | 2.42 |
| RM115610 | 2.77 | 2.29 | 2.12 | 2.39 |
| RM115048 | 2.50 | 2.03 | 2.55 | 2.36 |
| RM115097 | 2.90 | 2.27 | 1.91 | 2.36 |
| RM115000 | 2.56 | 2.14 | 2.20 | 2.30 |
| RM114848 | 1.93 | 2.60 | 2.36 | 2.30 |
| RM115380 | 2.35 | 2.14 | 2.02 | 2.17 |
| RM114730 | 2.13 | 2.26 | 2.08 | 2.16 |
| RM115273 | 2.26 | 2.00 | 2.20 | 2.15 |
| RM114729 | 2.42 | 2.27 | 1.76 | 2.15 |
| RM115608 | 3.08 | 1.78 | 1.54 | 2.13 |
| RM114881 | 2.32 | 1.84 | 2.20 | 2.12 |
| RM114925 | 2.39 | 1.97 | 1.97 | 2.11 |
| RM115058 | 2.12 | 2.22 | 1.97 | 2.10 |
| RM115009 | 2.14 | 2.28 | 1.85 | 2.09 |
| RM115191 | 1.13 | 3.72 | 1.40 | 2.08 |
| RM114766 | 1.90 | 2.30 | 2.04 | 2.08 |
| RM115534 | 1.95 | 1.93 | 2.18 | 2.02 |
| RM114884 | 2.19 | 1.86 | 1.96 | 2.00 |
| RM115169 | 2.32 | 1.65 | 2.01 | 1.99 |
| RM115208 | 1.31 | 2.64 | 2.02 | 1.99 |
| RM114913 | 1.89 | 2.09 | 1.94 | 1.97 |
| RM114719 | 2.11 | 1.59 | 2.14 | 1.94 |
| RM115170 | 1.86 | 1.88 | 2.03 | 1.92 |
| RM115405 | 1.66 | 1.81 | 2.27 | 1.91 |
| RM115075 | 1.75 | 2.27 | 1.59 | 1.87 |
| RM115472 | 1.46 | 2.12 | 1.92 | 1.83 |
| RM115585 | 1.46 | 2.31 | 1.68 | 1.82 |
| RM115249 | 1.77 | 2.06 | 1.57 | 1.80 |
| RM115248 | 1.17 | 1.95 | 2.27 | 1.80 |
| RM115451 | 1.97 | 2.05 | 1.29 | 1.77 |
| RM115614 | 1.80 | 1.87 | 1.57 | 1.75 |
| RM115037 | 0.74 | 2.81 | 1.68 | 1.74 |
| RM115353 | 1.53 | 1.69 | 1.92 | 1.71 |
| RM115181 | 1.62 | 1.63 | 1.78 | 1.68 |
| RM115649 | 2.20 | 1.85 | 0.96 | 1.67 |
| RM115551 | 1.92 | 1.27 | 1.79 | 1.66 |
| RM115039 | 1.55 | 1.69 | 1.71 | 1.65 |
| RM115435 | 1.52 | 1.88 | 1.36 | 1.59 |
| RM114743 | 1.70 | 1.49 | 1.56 | 1.58 |
| RM115188 | 1.82 | 1.45 | 1.47 | 1.58 |
| RM115329 | 1.60 | 1.29 | 1.78 | 1.56 |
| RM115460 | 1.31 | 1.69 | 1.63 | 1.55 |
| RM115143 | 1.44 | 1.29 | 1.82 | 1.52 |
| RM114726 | 1.54 | 1.02 | 1.97 | 1.51 |
| RM115162 | 1.30 | 1.58 | 1.64 | 1.51 |
| RM115420 | 1.27 | 1.50 | 1.70 | 1.49 |
| RM114898 | 1.38 | 1.56 | 1.50 | 1.48 |
| RM115196 | 1.79 | 1.17 | 1.45 | 1.47 |
| RM115270 | 1.53 | 1.44 | 1.40 | 1.46 |
| RM115174 | 1.37 | 1.81 | 1.17 | 1.45 |
| RM115182 | 1.65 | 1.48 | 1.20 | 1.44 |
| RM115612 | 1.97 | 1.00 | 1.36 | 1.44 |
| RM114835 | 1.33 | 1.54 | 1.37 | 1.41 |
| RM115013 | 1.23 | 1.58 | 1.41 | 1.41 |
| RM115244 | 1.11 | 1.90 | 1.12 | 1.38 |
| RM115082 | 1.88 | 1.34 | 0.85 | 1.35 |
| RM115120 | 1.96 | 1.44 | 0.63 | 1.34 |
| RM114892 | 1.07 | 1.73 | 1.21 | 1.34 |
| RM114873 | 1.24 | 1.32 | 1.45 | 1.34 |
| RM114745 | 1.51 | 1.13 | 1.35 | 1.33 |
| RM115278 | 1.03 | 1.74 | 1.13 | 1.30 |
| RM114930 | 1.46 | 1.43 | 1.01 | 1.30 |
| RM115600 | 1.44 | 1.28 | 1.15 | 1.29 |
| RM115532 | 1.11 | 1.17 | 1.57 | 1.28 |
| RM115150 | 1.61 | 1.04 | 1.20 | 1.28 |
| RM114757 | 1.53 | 1.08 | 1.22 | 1.28 |
| RM114882 | 0.89 | 1.00 | 0.81 | 0.90 |
| RM115543 | 0.74 | 0.90 | 1.06 | 0.90 |
| RM115060 | 0.99 | 0.73 | 0.99 | 0.90 |
| RM115068 | 0.78 | 1.41 | 0.51 | 0.90 |
| RM114786 | 1.03 | 0.94 | 0.72 | 0.90 |
| RM115061 | 0.72 | 1.01 | 0.95 | 0.89 |
| RM115087 | 0.58 | 1.06 | 1.03 | 0.89 |
| RM114720 | 0.96 | 0.50 | 1.21 | 0.89 |
| RM115524 | 1.17 | 0.76 | 0.72 | 0.89 |
| RM115044 | 0.38 | 1.52 | 0.75 | 0.88 |
| RM115306 | 0.45 | 1.36 | 0.83 | 0.88 |
| RM115053 | 1.00 | 0.69 | 0.94 | 0.88 |
| RM114758 | 0.95 | 0.94 | 0.73 | 0.87 |
| RM114844 | 0.95 | 0.76 | 0.90 | 0.87 |
| RM114976 | 0.67 | 1.08 | 0.84 | 0.86 |
| RM115445 | 0.85 | 0.78 | 0.92 | 0.85 |
| RM115043 | 0.88 | 0.91 | 0.75 | 0.85 |
| RM115129 | 0.90 | 0.70 | 0.93 | 0.84 |
| RM115025 | 1.11 | 0.61 | 0.81 | 0.84 |
| RM115354 | 0.90 | 0.83 | 0.80 | 0.84 |
| RM114883 | 1.11 | 0.58 | 0.84 | 0.84 |
| RM115604 | 0.56 | 1.05 | 0.92 | 0.84 |
| RM115475 | 0.94 | 0.81 | 0.78 | 0.84 |
| RM115117 | 1.02 | 0.76 | 0.74 | 0.84 |
| RM115277 | 1.03 | 0.80 | 0.68 | 0.84 |
| RM115165 | 0.76 | 0.26 | 1.47 | 0.83 |
| RM114762 | 0.67 | 1.29 | 0.53 | 0.83 |
| RM114987 | 0.99 | 0.70 | 0.75 | 0.81 |
| RM114737 | 0.99 | 0.80 | 0.63 | 0.81 |
| RM115064 | 0.70 | 0.70 | 1.02 | 0.81 |
| RM114951 | 0.97 | 0.72 | 0.72 | 0.80 |
| RM114775 | 0.67 | 0.89 | 0.83 | 0.80 |
| RM115160 | 0.29 | 0.91 | 1.19 | 0.80 |
| RM115190 | 0.72 | 0.71 | 0.95 | 0.79 |
| RM115446 | 0.59 | 0.81 | 0.98 | 0.79 |
| RM114734 | 0.64 | 1.01 | 0.72 | 0.79 |
| RM114969 | 0.93 | 0.77 | 0.67 | 0.79 |
| RM115398 | 1.14 | 0.49 | 0.73 | 0.79 |
| RM114858 | 0.70 | 0.99 | 0.68 | 0.79 |
| RM115230 | 1.14 | 0.57 | 0.65 | 0.79 |
| RM115313 | 0.74 | 0.56 | 1.05 | 0.79 |
| RM115161 | 1.05 | 0.70 | 0.61 | 0.79 |
| RM115206 | 0.90 | 0.76 | 0.70 | 0.78 |
| RM114964 | 0.97 | 0.58 | 0.80 | 0.78 |
| RM115547 | 1.29 | 0.59 | 0.46 | 0.78 |
| RM115173 | 0.64 | 0.96 | 0.74 | 0.78 |
| RM114866 | 0.68 | 0.78 | 0.85 | 0.77 |
| RM115175 | 0.78 | 0.72 | 0.78 | 0.76 |
| RM115184 | 0.67 | 0.72 | 0.88 | 0.76 |
| RM115052 | 0.49 | 0.83 | 0.94 | 0.75 |
| RM115399 | 0.53 | 0.92 | 0.78 | 0.74 |
| RM115454 | 1.00 | 0.65 | 0.58 | 0.74 |
| RM115374 | 0.65 | 0.71 | 0.86 | 0.74 |
| RM115309 | 0.62 | 0.90 | 0.68 | 0.73 |
| RM114837 | 0.72 | 0.62 | 0.86 | 0.73 |
| RM114917 | 0.65 | 1.08 | 0.46 | 0.73 |
| RM115026 | 0.84 | 0.78 | 0.56 | 0.73 |
| RM114915 | 0.67 | 0.89 | 0.62 | 0.73 |
| RM115142 | 0.86 | 0.63 | 0.69 | 0.73 |
| RM115392 | 0.44 | 0.44 | 1.29 | 0.72 |
| RM114760 | 0.76 | 0.71 | 0.70 | 0.72 |
| RM115063 | 0.80 | 0.68 | 0.68 | 0.72 |
| RM115334 | 0.65 | 0.80 | 0.72 | 0.72 |
| RM115197 | 1.03 | 0.48 | 0.66 | 0.72 |
| RM115016 | 0.79 | 0.58 | 0.79 | 0.72 |
| RM114926 | 0.90 | 0.62 | 0.63 | 0.72 |
| RM115457 | 0.48 | 0.61 | 1.06 | 0.72 |
| RM114989 | 0.72 | 0.81 | 0.60 | 0.71 |
| RM114714 | 0.76 | 0.71 | 0.64 | 0.71 |
| RM115219 | 0.59 | 0.65 | 0.54 | 0.59 |
| RM115455 | 0.65 | 0.45 | 0.67 | 0.59 |
| RM115357 | 0.62 | 0.45 | 0.69 | 0.59 |
| RM114943 | 0.46 | 0.90 | 0.41 | 0.59 |
| RM114865 | 0.60 | 0.58 | 0.58 | 0.59 |
| RM115350 | 0.66 | 0.47 | 0.63 | 0.59 |
| RM115437 | 0.68 | 0.45 | 0.62 | 0.58 |
| RM114713 | 0.64 | 0.65 | 0.46 | 0.58 |
| RM114901 | 0.50 | 0.63 | 0.61 | 0.58 |
| RM114997 | 0.53 | 0.47 | 0.73 | 0.58 |
| RM115032 | 0.41 | 0.60 | 0.71 | 0.58 |
| RM114872 | 0.54 | 0.55 | 0.64 | 0.57 |
| RM115429 | 0.55 | 0.55 | 0.61 | 0.57 |
| RM114800 | 0.66 | 0.41 | 0.64 | 0.57 |
| RM115284 | 0.67 | 0.60 | 0.43 | 0.57 |
| RM115320 | 0.70 | 0.47 | 0.53 | 0.57 |
| RM114778 | 0.60 | 0.43 | 0.66 | 0.56 |
| RM115259 | 0.33 | 0.58 | 0.78 | 0.56 |
| RM115077 | 0.72 | 0.55 | 0.41 | 0.56 |
| RM115041 | 0.53 | 0.47 | 0.69 | 0.56 |
| RM115033 | 0.75 | 0.57 | 0.37 | 0.56 |
| RM114841 | 0.79 | 0.56 | 0.34 | 0.56 |
| RM115049 | 0.70 | 0.58 | 0.39 | 0.56 |
| RM115338 | 0.51 | 0.55 | 0.62 | 0.56 |
| RM114876 | 0.70 | 0.61 | 0.36 | 0.56 |
| RM114918 | 0.32 | 0.82 | 0.52 | 0.56 |
| RM115355 | 0.56 | 0.70 | 0.40 | 0.56 |
| RM115484 | 0.36 | 0.79 | 0.51 | 0.55 |
| RM115494 | 0.55 | 0.29 | 0.81 | 0.55 |
| RM114825 | 0.55 | 0.55 | 0.55 | 0.55 |
| RM115285 | 0.56 | 0.59 | 0.49 | 0.55 |
| RM114912 | 0.53 | 0.44 | 0.67 | 0.55 |
| RM114877 | 0.46 | 0.62 | 0.56 | 0.55 |
| RM115176 | 0.58 | 0.58 | 0.47 | 0.55 |
| RM115106 | 0.79 | 0.50 | 0.34 | 0.54 |
| RM114793 | 0.59 | 0.40 | 0.64 | 0.54 |
| RM114722 | 0.64 | 0.30 | 0.68 | 0.54 |
| RM114871 | 0.36 | 0.62 | 0.63 | 0.54 |
| RM115021 | 0.63 | 0.67 | 0.31 | 0.54 |
| RM114823 | 0.51 | 0.39 | 0.71 | 0.54 |
| RM115466 | 0.29 | 0.38 | 0.95 | 0.54 |
| RM115453 | 0.53 | 0.52 | 0.56 | 0.54 |
| RM115128 | 0.49 | 0.69 | 0.43 | 0.54 |
| RM115595 | 0.36 | 0.81 | 0.44 | 0.53 |
| RM114994 | 0.16 | 0.91 | 0.53 | 0.53 |
| RM114885 | 0.68 | 0.44 | 0.48 | 0.53 |
| RM115363 | 0.28 | 0.45 | 0.86 | 0.53 |
| RM115337 | 0.49 | 0.55 | 0.54 | 0.53 |
| RM115124 | 0.43 | 0.57 | 0.59 | 0.53 |
| RM115627 | 0.60 | 0.23 | 0.75 | 0.53 |
| RM115539 | 0.55 | 0.41 | 0.63 | 0.53 |
| RM115630 | 0.29 | 0.54 | 0.75 | 0.53 |
| RM115462 | 0.64 | 0.40 | 0.53 | 0.53 |
| RM115376 | 0.36 | 0.49 | 0.72 | 0.52 |
| RM115239 | 0.63 | 0.65 | 0.29 | 0.52 |
| RM115266 | 0.57 | 0.36 | 0.64 | 0.52 |
| RM115198 | 0.47 | 0.52 | 0.58 | 0.52 |
| RM115122 | 0.48 | 0.51 | 0.58 | 0.52 |
| RM115089 | 0.30 | 1.10 | 0.16 | 0.52 |
| RM115059 | 0.40 | 0.50 | 0.65 | 0.52 |
| RM115465 | 0.48 | 0.57 | 0.50 | 0.52 |
| RM115080 | 0.51 | 0.51 | 0.54 | 0.52 |
| RM115002 | 0.33 | 0.71 | 0.50 | 0.52 |
| RM115231 | 0.59 | 0.64 | 0.31 | 0.52 |
| RM115054 | 0.54 | 0.47 | 0.54 | 0.52 |
| RM115189 | 0.55 | 0.53 | 0.45 | 0.51 |
| RM114852 | 0.54 | 0.49 | 0.51 | 0.51 |
| RM114759 | 0.38 | 0.45 | 0.70 | 0.51 |
| RM115598 | 0.54 | 0.46 | 0.53 | 0.51 |
| RM115596 | 0.53 | 0.47 | 0.29 | 0.43 |
| RM115006 | 0.48 | 0.44 | 0.38 | 0.43 |
| RM115560 | 0.53 | 0.59 | 0.16 | 0.43 |
| RM114772 | 0.48 | 0.38 | 0.42 | 0.43 |
| RM115028 | 0.50 | 0.52 | 0.26 | 0.43 |
| RM115291 | 0.38 | 0.32 | 0.58 | 0.43 |
| RM114977 | 0.45 | 0.36 | 0.47 | 0.42 |
| RM115321 | 0.33 | 0.52 | 0.41 | 0.42 |
| RM115302 | 0.48 | 0.26 | 0.51 | 0.42 |
| RM114953 | 0.09 | 0.83 | 0.32 | 0.41 |
| RM114831 | 0.38 | 0.42 | 0.44 | 0.41 |
| RM114824 | 0.38 | 0.55 | 0.30 | 0.41 |
| RM115546 | 0.29 | 0.29 | 0.65 | 0.41 |
| RM114965 | 0.43 | 0.43 | 0.36 | 0.41 |
| RM114962 | 0.48 | 0.25 | 0.49 | 0.41 |
| RM115426 | 0.39 | 0.17 | 0.66 | 0.41 |
| RM115336 | 0.28 | 0.37 | 0.57 | 0.40 |
| RM115468 | 0.54 | 0.31 | 0.36 | 0.40 |
| RM115384 | 0.34 | 0.54 | 0.33 | 0.40 |
| RM114942 | 0.46 | 0.36 | 0.39 | 0.40 |
| RM114948 | 0.21 | 0.47 | 0.53 | 0.40 |
| RM115572 | 0.43 | 0.40 | 0.37 | 0.40 |
| RM114707 | 0.65 | 0.33 | 0.23 | 0.40 |
| RM114959 | 0.48 | 0.44 | 0.28 | 0.40 |
| RM115029 | 0.38 | 0.42 | 0.40 | 0.40 |
| RM114900 | 0.31 | 0.41 | 0.47 | 0.40 |
| RM114826 | 0.10 | 0.24 | 0.85 | 0.40 |
| RM114849 | 0.55 | 0.32 | 0.32 | 0.40 |
| RM115372 | 0.41 | 0.51 | 0.27 | 0.40 |
| RM114911 | 0.31 | 0.45 | 0.42 | 0.40 |
| RM115138 | 0.40 | 0.41 | 0.36 | 0.39 |
| RM115069 | 0.47 | 0.30 | 0.41 | 0.39 |
| RM114750 | 0.31 | 0.48 | 0.39 | 0.39 |
| RM115040 | 0.38 | 0.35 | 0.44 | 0.39 |
| RM115263 | 0.38 | 0.36 | 0.43 | 0.39 |
| RM114980 | 0.37 | 0.31 | 0.48 | 0.39 |
| RM115488 | 0.31 | 0.39 | 0.46 | 0.38 |
| RM115388 | 0.33 | 0.44 | 0.39 | 0.38 |
| RM115417 | 0.45 | 0.40 | 0.31 | 0.38 |
| RM115521 | 0.36 | 0.33 | 0.45 | 0.38 |
| RM114973 | 0.46 | 0.28 | 0.40 | 0.38 |
| RM115274 | 0.39 | 0.25 | 0.48 | 0.38 |
| RM115390 | 0.40 | 0.40 | 0.33 | 0.38 |
| RM115102 | 0.49 | 0.27 | 0.37 | 0.37 |
| RM115134 | 0.46 | 0.31 | 0.35 | 0.37 |
| RM114938 | 0.34 | 0.49 | 0.29 | 0.37 |
| RM114971 | 0.41 | 0.28 | 0.43 | 0.37 |
| RM115235 | 0.35 | 0.45 | 0.32 | 0.37 |
| RM115237 | 0.42 | 0.36 | 0.33 | 0.37 |
| RM115483 | 0.28 | 0.40 | 0.42 | 0.37 |
| RM115216 | 0.26 | 0.44 | 0.40 | 0.37 |
| RM114939 | 0.32 | 0.39 | 0.39 | 0.37 |
| RM115395 | 0.33 | 0.44 | 0.32 | 0.36 |
| RM115628 | 0.28 | 0.61 | 0.19 | 0.36 |
| RM115139 | 0.40 | 0.38 | 0.30 | 0.36 |
| RM114817 | 0.15 | 0.44 | 0.48 | 0.36 |
| RM114833 | 0.47 | 0.39 | 0.21 | 0.36 |
| RM114929 | 0.33 | 0.39 | 0.34 | 0.35 |
| RM114832 | 0.11 | 0.45 | 0.50 | 0.35 |
| RM114860 | 0.30 | 0.11 | 0.64 | 0.35 |
| RM115553 | 0.14 | 0.63 | 0.27 | 0.35 |
| RM114940 | 0.22 | 0.56 | 0.27 | 0.35 |
| RM115415 | 0.38 | 0.30 | 0.36 | 0.35 |
| RM115505 | 0.24 | 0.45 | 0.34 | 0.34 |
| RM115294 | 0.30 | 0.41 | 0.31 | 0.34 |
| RM114963 | 0.30 | 0.48 | 0.25 | 0.34 |
| RM115298 | 0.18 | 0.46 | 0.38 | 0.34 |
| RM115103 | 0.32 | 0.26 | 0.45 | 0.34 |
| RM115396 | 0.39 | 0.37 | 0.26 | 0.34 |
| RM114705 | 0.20 | 0.35 | 0.32 | 0.29 |
| RM115650 | 0.14 | 0.48 | 0.26 | 0.29 |
| RM115589 | 0.28 | 0.42 | 0.17 | 0.29 |
| RM114923 | 0.18 | 0.41 | 0.28 | 0.29 |
| RM114712 | 0.43 | 0.20 | 0.24 | 0.29 |
| RM114984 | 0.23 | 0.32 | 0.32 | 0.29 |
| RM115367 | 0.44 | 0.18 | 0.25 | 0.29 |
| RM114879 | 0.24 | 0.33 | 0.29 | 0.29 |
| RM115584 | 0.40 | 0.27 | 0.19 | 0.29 |
| RM115371 | 0.32 | 0.26 | 0.28 | 0.29 |
| RM115217 | 0.35 | 0.23 | 0.27 | 0.29 |
| RM115246 | 0.58 | 0.17 | 0.10 | 0.28 |
| RM115118 | 0.33 | 0.20 | 0.31 | 0.28 |
| RM114863 | 0.17 | 0.58 | 0.09 | 0.28 |
| RM115391 | 0.16 | 0.36 | 0.33 | 0.28 |
| RM115480 | 0.29 | 0.33 | 0.23 | 0.28 |
| RM115487 | 0.26 | 0.45 | 0.13 | 0.28 |
| RM114768 | 0.38 | 0.16 | 0.30 | 0.28 |
| RM114906 | 0.32 | 0.39 | 0.13 | 0.28 |
| RM115646 | 0.34 | 0.33 | 0.15 | 0.28 |
| RM114795 | 0.19 | 0.24 | 0.40 | 0.28 |
| RM114773 | 0.23 | 0.38 | 0.22 | 0.28 |
| RM114931 | 0.22 | 0.31 | 0.29 | 0.28 |
| RM115300 | 0.25 | 0.26 | 0.31 | 0.27 |
| RM114808 | 0.36 | 0.19 | 0.26 | 0.27 |
| RM115228 | 0.25 | 0.23 | 0.34 | 0.27 |
| RM115479 | 0.39 | 0.19 | 0.24 | 0.27 |
| RM115115 | 0.27 | 0.31 | 0.24 | 0.27 |
| RM115094 | 0.13 | 0.35 | 0.33 | 0.27 |
| RM115157 | 0.22 | 0.28 | 0.30 | 0.27 |
| RM115378 | 0.29 | 0.26 | 0.25 | 0.26 |
| RM114920 | 0.06 | 0.24 | 0.49 | 0.26 |
| RM115499 | 0.24 | 0.42 | 0.13 | 0.26 |
| RM115346 | 0.27 | 0.26 | 0.26 | 0.26 |
| RM114992 | 0.18 | 0.32 | 0.29 | 0.26 |
| RM114813 | 0.28 | 0.25 | 0.26 | 0.26 |
| RM115545 | 0.27 | 0.24 | 0.28 | 0.26 |
| RM115522 | 0.23 | 0.23 | 0.32 | 0.26 |
| RM115400 | 0.31 | 0.30 | 0.18 | 0.26 |
| RM115130 | 0.13 | 0.25 | 0.39 | 0.26 |
| RM115345 | 0.17 | 0.31 | 0.28 | 0.26 |
| RM115436 | 0.22 | 0.40 | 0.14 | 0.25 |
| RM115648 | 0.24 | 0.15 | 0.36 | 0.25 |
| RM114804 | 0.20 | 0.37 | 0.18 | 0.25 |
| RM114708 | 0.34 | 0.20 | 0.22 | 0.25 |
| RM115433 | 0.18 | 0.30 | 0.27 | 0.25 |
| RM114949 | 0.33 | 0.17 | 0.25 | 0.25 |
| RM115361 | 0.31 | 0.17 | 0.26 | 0.25 |
| RM115100 | 0.19 | 0.37 | 0.19 | 0.25 |
| RM115370 | 0.32 | 0.31 | 0.11 | 0.25 |
| RM115098 | 0.29 | 0.21 | 0.24 | 0.25 |
| RM115449 | 0.44 | 0.15 | 0.15 | 0.25 |
| RM114803 | 0.30 | 0.22 | 0.21 | 0.24 |
| RM115215 | 0.18 | 0.31 | 0.24 | 0.24 |
| RM114904 | 0.54 | 0.13 | 0.06 | 0.24 |
| RM115067 | 0.09 | 0.37 | 0.27 | 0.24 |
| RM114982 | 0.07 | 0.16 | 0.49 | 0.24 |
| RM115292 | 0.20 | 0.19 | 0.32 | 0.24 |
| RM114799 | 0.22 | 0.18 | 0.32 | 0.24 |
| RM115511 | 0.24 | 0.31 | 0.16 | 0.24 |
| RM115255 | 0.31 | 0.21 | 0.18 | 0.23 |
| RM115104 | 0.20 | 0.24 | 0.26 | 0.23 |
| RM114836 | 0.42 | 0.00 | 0.28 | 0.23 |
| RM115529 | 0.18 | 0.34 | 0.18 | 0.23 |
| RM115482 | 0.23 | 0.33 | 0.13 | 0.23 |
| RM115344 | 0.17 | 0.24 | 0.27 | 0.23 |
| RM115257 | 0.21 | 0.24 | 0.24 | 0.23 |
| RM114765 | 0.20 | 0.23 | 0.26 | 0.23 |
| RM114820 | 0.27 | 0.22 | 0.19 | 0.23 |
| RM114839 | 0.13 | 0.20 | 0.18 | 0.17 |
| RM114998 | 0.18 | 0.20 | 0.14 | 0.17 |
| RM115407 | 0.14 | 0.23 | 0.13 | 0.17 |
| RM115091 | 0.28 | 0.10 | 0.13 | 0.17 |
| RM114779 | 0.36 | 0.07 | 0.07 | 0.17 |
| RM114887 | 0.08 | 0.19 | 0.24 | 0.17 |
| RM114789 | 0.13 | 0.14 | 0.23 | 0.17 |
| RM115508 | 0.13 | 0.17 | 0.19 | 0.17 |
| RM115570 | 0.09 | 0.23 | 0.18 | 0.17 |
| RM115501 | 0.19 | 0.06 | 0.25 | 0.16 |
| RM115011 | 0.12 | 0.20 | 0.17 | 0.16 |
| RM115319 | 0.22 | 0.13 | 0.14 | 0.16 |
| RM115448 | 0.11 | 0.19 | 0.19 | 0.16 |
| RM115507 | 0.31 | 0.08 | 0.10 | 0.16 |
| RM114787 | 0.33 | 0.00 | 0.16 | 0.16 |
| RM114952 | 0.22 | 0.12 | 0.15 | 0.16 |
| RM114780 | 0.09 | 0.19 | 0.19 | 0.16 |
| RM115035 | 0.19 | 0.13 | 0.17 | 0.16 |
| RM114886 | 0.12 | 0.22 | 0.14 | 0.16 |
| RM115587 | 0.19 | 0.14 | 0.15 | 0.16 |
| RM115583 | 0.17 | 0.26 | 0.04 | 0.16 |
| RM115594 | 0.18 | 0.11 | 0.19 | 0.16 |
| RM114746 | 0.11 | 0.16 | 0.20 | 0.16 |
| RM115506 | 0.16 | 0.13 | 0.17 | 0.16 |
| RM115186 | 0.12 | 0.23 | 0.12 | 0.16 |
| RM115478 | 0.20 | 0.22 | 0.05 | 0.16 |
| RM114718 | 0.12 | 0.17 | 0.18 | 0.16 |
| RM114736 | 0.12 | 0.22 | 0.12 | 0.15 |
| RM115364 | 0.19 | 0.20 | 0.07 | 0.15 |
| RM115563 | 0.10 | 0.18 | 0.19 | 0.15 |
| RM115359 | 0.07 | 0.39 | 0.00 | 0.15 |
| RM114958 | 0.12 | 0.09 | 0.25 | 0.15 |
| RM115599 | 0.10 | 0.21 | 0.15 | 0.15 |
| RM114815 | 0.22 | 0.09 | 0.15 | 0.15 |
| RM115095 | 0.19 | 0.08 | 0.17 | 0.15 |
| RM114716 | 0.22 | 0.02 | 0.21 | 0.15 |
| RM115509 | 0.07 | 0.12 | 0.25 | 0.15 |
| RM115408 | 0.07 | 0.15 | 0.22 | 0.15 |
| RM115121 | 0.09 | 0.14 | 0.22 | 0.15 |
| RM115116 | 0.13 | 0.26 | 0.06 | 0.15 |
| RM115096 | 0.19 | 0.21 | 0.05 | 0.15 |
| RM115267 | 0.18 | 0.12 | 0.14 | 0.14 |
| RM115481 | 0.17 | 0.14 | 0.12 | 0.14 |
| RM115653 | 0.14 | 0.13 | 0.16 | 0.14 |
| RM114830 | 0.09 | 0.19 | 0.15 | 0.14 |
| RM114770 | 0.16 | 0.09 | 0.19 | 0.14 |
| RM115527 | 0.11 | 0.25 | 0.07 | 0.14 |
| RM115147 | 0.09 | 0.25 | 0.09 | 0.14 |
| RM115443 | 0.17 | 0.10 | 0.15 | 0.14 |
| RM114761 | 0.15 | 0.21 | 0.07 | 0.14 |
| RM115222 | 0.13 | 0.16 | 0.13 | 0.14 |
| RM114909 | 0.15 | 0.11 | 0.16 | 0.14 |
| RM115626 | 0.07 | 0.12 | 0.22 | 0.14 |
| RM114703 | 0.18 | 0.18 | 0.05 | 0.14 |
| RM115590 | 0.13 | 0.15 | 0.13 | 0.14 |
| RM115622 | 0.11 | 0.17 | 0.13 | 0.14 |
| RM114878 | 0.09 | 0.13 | 0.19 | 0.14 |
| RM114867 | 0.11 | 0.14 | 0.15 | 0.13 |
| RM115418 | 0.18 | 0.22 | 0.00 | 0.13 |
| RM115410 | 0.14 | 0.06 | 0.19 | 0.13 |
| RM114709 | 0.14 | 0.15 | 0.10 | 0.13 |
| RM115276 | 0.11 | 0.16 | 0.12 | 0.13 |
| RM115112 | 0.16 | 0.14 | 0.09 | 0.13 |
| RM115201 | 0.10 | 0.05 | 0.24 | 0.13 |
| RM114946 | 0.13 | 0.19 | 0.08 | 0.13 |
| RM115574 | 0.11 | 0.15 | 0.13 | 0.13 |
| RM115576 | 0.12 | 0.13 | 0.13 | 0.13 |
| RM115523 | 0.22 | 0.08 | 0.09 | 0.13 |
| RM114821 | 0.10 | 0.17 | 0.12 | 0.13 |
| RM114981 | 0.07 | 0.13 | 0.09 | 0.09 |
| RM114907 | 0.10 | 0.07 | 0.11 | 0.09 |
| RM115530 | 0.12 | 0.13 | 0.04 | 0.09 |
| RM114828 | 0.04 | 0.18 | 0.06 | 0.09 |
| RM115629 | 0.12 | 0.10 | 0.07 | 0.09 |
| RM114927 | 0.11 | 0.06 | 0.11 | 0.09 |
| RM115280 | 0.10 | 0.09 | 0.07 | 0.09 |
| RM115295 | 0.11 | 0.09 | 0.06 | 0.09 |
| RM114700 | 0.09 | 0.06 | 0.10 | 0.09 |
| RM115469 | 0.11 | 0.14 | 0.00 | 0.08 |
| RM114784 | 0.13 | 0.06 | 0.06 | 0.08 |
| RM114796 | 0.05 | 0.12 | 0.07 | 0.08 |
| RM115238 | 0.06 | 0.09 | 0.09 | 0.08 |
| RM115108 | 0.07 | 0.11 | 0.06 | 0.08 |
| RM115042 | 0.13 | 0.11 | 0.00 | 0.08 |
| RM115254 | 0.04 | 0.10 | 0.10 | 0.08 |
| RM115167 | 0.12 | 0.04 | 0.07 | 0.08 |
| RM115636 | 0.10 | 0.07 | 0.07 | 0.08 |
| RM115510 | 0.03 | 0.06 | 0.14 | 0.08 |
| RM114935 | 0.11 | 0.05 | 0.08 | 0.08 |
| RM115386 | 0.08 | 0.11 | 0.05 | 0.08 |
| RM115126 | 0.00 | 0.10 | 0.13 | 0.08 |
| RM114921 | 0.10 | 0.08 | 0.05 | 0.08 |
| RM115164 | 0.05 | 0.13 | 0.05 | 0.08 |
| RM114725 | 0.14 | 0.09 | 0.00 | 0.08 |
| RM115557 | 0.07 | 0.12 | 0.04 | 0.08 |
| RM115489 | 0.07 | 0.06 | 0.10 | 0.07 |
| RM115268 | 0.13 | 0.06 | 0.02 | 0.07 |
| RM115644 | 0.05 | 0.09 | 0.07 | 0.07 |
| RM115221 | 0.06 | 0.09 | 0.07 | 0.07 |
| RM115131 | 0.12 | 0.00 | 0.10 | 0.07 |
| RM114854 | 0.04 | 0.07 | 0.11 | 0.07 |
| RM115620 | 0.05 | 0.06 | 0.10 | 0.07 |
| RM114908 | 0.06 | 0.10 | 0.05 | 0.07 |
| RM115623 | 0.10 | 0.08 | 0.03 | 0.07 |
| RM115383 | 0.05 | 0.11 | 0.04 | 0.07 |
| RM115618 | 0.05 | 0.09 | 0.06 | 0.07 |
| RM115542 | 0.10 | 0.07 | 0.03 | 0.06 |
| RM114894 | 0.00 | 0.08 | 0.11 | 0.06 |
| RM115637 | 0.06 | 0.05 | 0.08 | 0.06 |
| RM115550 | 0.06 | 0.04 | 0.09 | 0.06 |
| RM115565 | 0.11 | 0.02 | 0.06 | 0.06 |
| RM115566 | 0.00 | 0.07 | 0.12 | 0.06 |
| RM114710 | 0.04 | 0.07 | 0.07 | 0.06 |
| RM115642 | 0.04 | 0.09 | 0.05 | 0.06 |
| RM115425 | 0.04 | 0.00 | 0.13 | 0.06 |
| RM114811 | 0.03 | 0.13 | 0.00 | 0.05 |
| RM114966 | 0.09 | 0.00 | 0.07 | 0.05 |
| RM114816 | 0.00 | 0.07 | 0.08 | 0.05 |
| RM115490 | 0.05 | 0.00 | 0.10 | 0.05 |
| RM115640 | 0.04 | 0.03 | 0.08 | 0.05 |
| RM114974 | 0.03 | 0.06 | 0.06 | 0.05 |
| RM115617 | 0.05 | 0.06 | 0.04 | 0.05 |
| RM114875 | 0.05 | 0.09 | 0.00 | 0.05 |
| RM115036 | 0.04 | 0.04 | 0.05 | 0.05 |
| RM115568 | 0.00 | 0.00 | 0.14 | 0.05 |
| RM114845 | 0.00 | 0.13 | 0.00 | 0.04 |
| RM115305 | 0.00 | 0.13 | 0.00 | 0.04 |
| RM115562 | 0.03 | 0.04 | 0.05 | 0.04 |
| RM115538 | 0.06 | 0.04 | 0.02 | 0.04 |
| RM115166 | 0.00 | 0.00 | 0.12 | 0.04 |
| RM115114 | 0.00 | 0.09 | 0.00 | 0.03 |
| RM114934 | 0.00 | 0.00 | 0.00 | 0.00 |

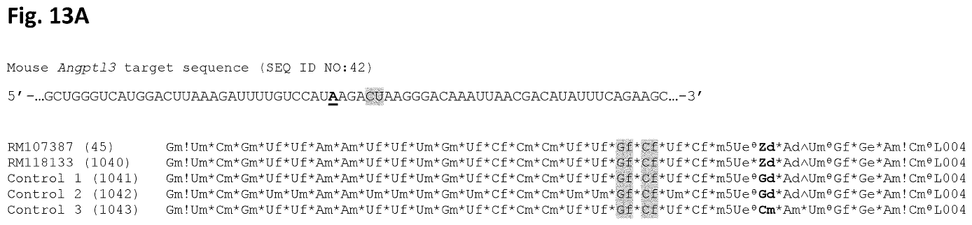

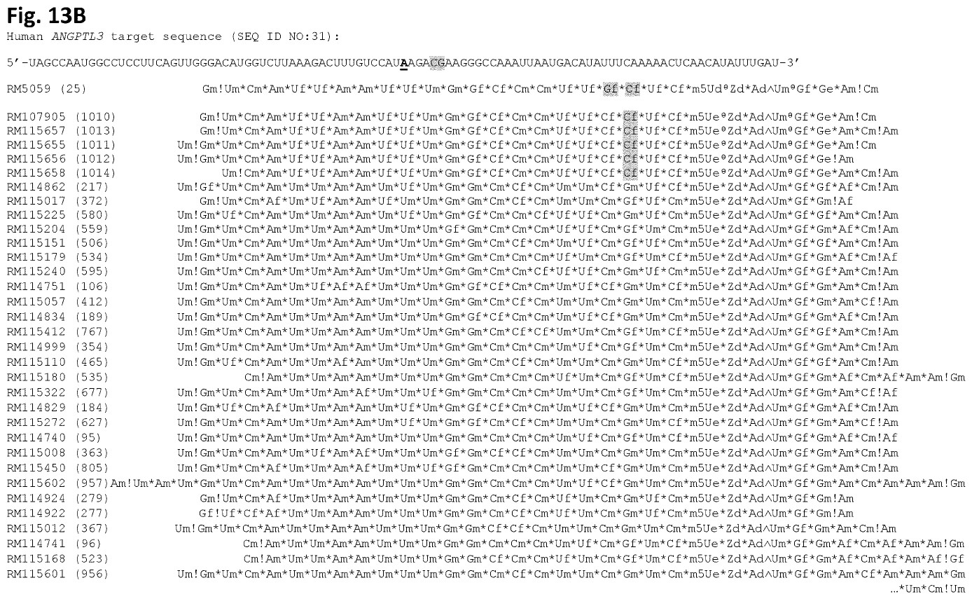

EONs RM107905 (SEQ ID NO:1010), RM115657 (SEQ ID NO:1013), RM115655 (SEQ ID NO: 1011), RM115656 (SEQ ID NO: 1012), RM115658 (SEQ ID NO:1014), RM114862 (SEQ ID NO: 217), RM115017 (SEQ ID NO:372), RM115225 (SEQ ID NO:580), RM115204 (SEQ ID NO: 559), RM115151 (SEQ ID NO:506), RM115179 (SEQ ID NO:534), RM115240 (SEQ ID NO: 595), RM114751 (SEQ ID NO: 106), RM115057 (SEQ ID NO:412), RM114834 (SEQ ID NO: 189), RM115412 (SEQ ID NO:767), RM114999 (SEQ ID NO:345), RM115110 (SEQ ID NO: 465), RM115180 (SEQ ID NO:535), RM115322 (SEQ ID NO:677), RM114829 (SEQ ID NO: 184), RM115272 (SEQ ID NO:627), RM114740 (SEQ ID NO:95), RM115008 (SEQ ID NO: 363), RM115450 (SEQ ID NO:805), RM115602 (SEQ ID NO:957), RM114924 (SEQ ID NO: 279), RM114922 (SEQ ID NO:277), RM115012 (SEQ ID NO:367), RM114741 (SEQ ID NO: 96), RM115168 (SEQ ID NO:523), and RM115601 (SEQ ID NO:956) that target the human ANGPTL3 transcript and that scored 5% or higher in editing (see Table 5) are again listed in FIG. 13B together with RM5059 (SEQ ID NO:25). There are two differences between RM5059 and RM107905: i) the 2′-MOE modification in the ribose of the 5-methyluridine nucleotide at position +1; and ii) nucleotide position +5, where RM5059 has a G:G mismatch with the target sequence and RM107905 makes a C:G complementary base pair. Interestingly, RM114862, RM115017, RM115225, RM115204, RM115151, and RM115179 (that all perform good, but not as good as the top 5 best performers in the screen) all have a G:C and a C:G Watson-Crick base pair in the EONs at positions +4 and +5, respectively, whereas the top 5 performers all comprise a C:C mismatch at position +4, indicating that a mismatch at this position is preferred to obtain higher editing percentages. Other features that RM115655, RM115656, RM115657, and RM115658 share with RM107905 are the PO linkages at linkage position 0 and −3 (in contrast to all other properly performing EONs that have PS at those positions), and the presence of a 2′-MOE modified G (Ge) at nucleotide position −4 (in contrast to all other properly performing EONs that have either Gm or Gf at that position). Strikingly, it appears that in any case an ‘a-symmetrical’ design in which there are more nucleotides at the 5′ side of the orphan nucleotide than there are at the 3′ side of the orphan nucleotide in the EON (opposite the area that is targeted in the human ANGPTL3 transcript molecule) performs better than ‘symmetrical’ designs in which the orphan nucleotide is positioned in the middle, or close to the middle of the EON. All 32 EONs that perform well in providing at least 5% editing in the high-throughput screen, together with RM5059, have such an a-symmetrical design, as shown in FIG. 13B. Besides that, the length (and the area of complementarity) of most EONs in FIG. 13B appears to have a tendency, with a length of 29-32 nucleotides being sufficient to obtain good editing. Hence, in a preferred embodiment, the EON as disclosed herein has an a-symmetrical design and is preferably 29, 30, 31, or 32 nucleotides in length, wherein the orphan nucleotide is nucleotide position 0 and there are preferably 5, 6, or 7 nucleotides at the 3′ side of the orphan nucleotide and preferably 22, 23, or 24 nucleotides at the 5′ side of the orphan nucleotide.

In a preferred embodiment, the EON of the present disclosure has the structure:

5′-N₂₄N₂₃N₂₂N₂₁N₂₀N₁₉N₁₈N₁₇N₁₆N₁₅N₁₄N₁₃N₁₂N₁₁N₁₀N₉N₈N₇N₆N₅N₄N₃
N₂N₁^{θ}ZdAd^{∧}M₂M₃M₄M₅M₆M₇-3′

wherein:
   Zd is the orphan nucleotide at nucleotide position 0, which is a deoxynucleotide carrying a Benner's base;
   N₁ is a thymidine (5-methyluridine with a deoxyribose; m5Ud), or a 5-methyluridine with a 2′-MOE ribose substitution (m5Ue);
   N₂ is a cytidine with a 2′-fluoro (Cf) or a 2′-OMe (Cm) ribose substitution;
   N₃ is a uridine with a 2′-fluoro (Uf) or a 2′-OMe (Um) ribose substitution;
   N₄ is a cytidine with a 2′-fluoro ribose substitution (Cf), or a guanosine with a 2′-fluoro (Gf) or a 2′-OMe (Gm) ribose substitution;
   N₅ is a guanosine with a 2′-fluoro ribose substitution (Gf), or a cytidine with a 2′-fluoro (Cf) or a 2′-OMe (Cm) ribose substitution;
   N₆, N₇, N₁₃, N₁₄, N₁₅, N₁₈, N₁₉, N₂₂, and M₂ are uridine with either a 2′-fluoro ribose substitution (Uf) or a 2′-OMe ribose substitution (Um);
   N₈, N₉, N₁₀, and N₂₁ are cytidine with either a 2′-fluoro ribose substitution (Cf) or a 2′-OMe ribose substitution (Cm);
   N₁₁, N₁₂, and M₃ are guanosine with either a 2′-fluoro ribose substitution (Gf) or a 2′-OMe ribose substitution (Gm);
   M₄ is guanosine with a 2′-fluoro ribose substitution (Gf), a 2′-OMe ribose substitution (Gm), or a 2′-MOE ribose substitution (Ge);
   N₁₆, N₁₇, N₂₀, and M₅ are adenosine with either a 2′-fluoro ribose substitution (Af) or a 2′-OMe ribose substitution (Am);
   N₂₃ and N₂₄ are absent; or N₂₃ is Gf or Gm, and N₂₄ is absent; or N₂₃ is Gf or Gm, and N₂₄ is Um;
   M₆ and M₇ are absent; or M₆ is Cm and M₇ is absent; or Me is Cm and M₇ is Am or Af;
   Ad is deoxyadenosine;
   θ is at linkage position 0, and is a PO linkage;
   \hat{ } is at linkage position −2 and is an MP or a PNms linkage;
   linkage positions-3 and 5 are, each independently, a PO or a PS linkage, preferably a PO linkage;
   linkage position +23 is a PNdmi or a PNms linkage;
   linkage position −7 is a PNdmi or a PNms linkage; and
   all other linkages are PS linkages.

### Example 8. In Vivo Editing of the Target Adenosine in Angptl3 Transcripts of Mice and Monkeys, and Effect on Biomarkers

In a next study, which resembles the in vivo study of example 5, RM107387 is compared to RM118133 (see FIG. 13A) in mice, to determine the in vivo effect of having a PS linkage (RM118133) at linkage position 0 in contrast to a PO linkage (RM107387). Next to these EONs, three negative control EONs are taken along, also shown in FIG. 13A: control 1, 2 and 3 (SEQ ID NO: 1041, 1042, and 1043, respectively). These control EONs comprise either a deoxyguanosine (Gd) or a 2′-OMe modified cytidine (Cm) at the orphan position, which should in principle prevent editing. Control 1 comprises a PO linkage at linkage position 0, whereas control 2 further comprises 2′-OMe modified uridines (Um) in the positions where RM107387 comprises 2′-fluoro modified uridines (Uf) to study off-target editing. These five EONs are tested in several C57BL/6J mice groups (8 mice each), with a variety of doses (10 and 50 mg/kg), and with a range of dosing regimens (dosing at day 0, 2, and 4, in comparison to dosing on day 0, 2, 4, 7, 14, 21, and 28 days). Necropsy is at day 7, 14, 21, or 31. Controls receive PBS. Welfare, weight, clinical chemistry, and haematology is assessed. Blood sampling is applied during the study and at necropsy, for pre- and post-heparin determination, and to determine levels of LDL, LPL-C and ApoB, like the study of example 5. After necropsy, liver and kidney samples are drawn to determine editing levels.

Further, the effect of RM107387 and RM118133 on editing, plasma lipids and LPL activity is studied in 15-17 weeks old female APOE*3Leiden.CETP transgenic mice (Princen HMG et al. 2016. Toxicol Rep. 3:306-309), which is a humanized model for familial dyslipoproteinemia (FD) and mixed dyslipoproteinemia. While normal wild-type mice have a very rapid clearance of apoB-containing lipoproteins, APOE*3-Leiden.CETP mice have an impaired clearance and increased TG level and are thereby mimicking the slow clearance observed in humans, particularly in patients with FD. Importantly, as compared to the widely used hyperlipidemic and atherogenic apoE- and LDLR-deficient (E0/0 and LDLR0/0) mice, the APOE*3-Leiden.CETP mice possess an intact but delayed apoE-LDLR-mediated clearance, which is essential for the proper, human-like response on hypolipidemic drugs. APOE*3-Leiden.CETP mice respond well to dietary intervention using human-relevant (Westernized) diets with increases in plasma cholesterol and TG and these lipids can be titrated to levels mimicking those in humans. Hence, initially the mice are set on a Western-type diet with 0.15% cholesterol to induce hyperlipidaemia. Editing of the endogenous Angptl3 transcript (as disclosed supra) is thought to influence the LPL activity and the levels of LDL-C in these mice. Dosing is performed with 10 and 50 mg/kg EON in different groups of 8 mice each. Dosing is performed on day 0, 2, 4, 7, 14, 21, and 28. Editing percentages in liver and kidney are measured as described in example 5. Measured are also body weight, food intake, plasma levels of triglycerides, FFA, LPL activity, LDL-C levels and ApoB, like the study of examples 5 and 8.

In yet a further study, an EON is tested in non-human primates that specifically targets the Angptl3 transcript in Cynomolgus monkey. This EON comprises a tri-antennary GalNAc moiety attached to the 3′ terminus for increased in vivo delivery to the liver. The EON (SEQ ID NO: 1044) has the following sequence and modifications (see legends to FIG. 8B):

5′-Gm!Um*Cm*Am*Uf*Uf*Am*Am*Uf*Uf*Um*Gm*Gf*Cf*Cm*Cm*Uf*

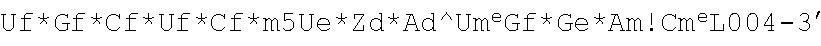

It should be noted that the grey boxed Gf is opposite a U in the monkey target sequence (in contrast to the presence of a G in the human target sequence), which is still a mismatch. The grey boxed Cf is opposite a C in the monkey target sequence (like what is seen in the human target sequence). Hence, this EON also has two mismatches with the target sequence (in monkey), but this is partly different from the two mismatches of for instance RM5059 (see above). In contrast to RM5059, RM107905, and RM107387, this EON comprises a PS linkage at linkage position 0. The EON is administered to Cynomolgus monkey(s), whereafter Angptl3 editing, and effects on downstream biomarkers (as discussed above) are studied.

1. An antisense oligonucleotide (EON) conducive to ADAR-mediated adenosine deamination and capable of forming a double-stranded complex with a region of an endogenous human ANGPTL3 pre-mRNA or mRNA molecule in a human cell, wherein the region of the ANGPTL3 pre-mRNA or mRNA molecule comprises a target adenosine, and wherein the double-stranded complex can recruit an endogenous ADAR1 and/or ADAR2 enzyme to deaminate the target adenosine into an inosine, thereby editing the ANGPTL3 pre-mRNA or mRNA molecule.

2. An EON according to claim 1, wherein the cell is a hepatocyte.

3. An EON according to claim 1 or 2, wherein the target adenosine is present in a codon encoding an amino acid involved in the lipase inhibition functionality of the ANGPTL3 protein, and wherein the deamination of the target adenosine into an inosine results in an ANGPTL3 protein that is impaired in its lipase inhibitory function.

4. An EON according to claim 3, wherein the target adenosine is the first nucleotide in the codon encoding lysine at position 63 in the human ANGPTL3 protein.

5. An EON according to any one of claims 1 to 4, wherein at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution.

6. An EON according to claim 5, wherein the one or more additional modifications in the linkage moiety is each independently selected from a phosphorothioate (PS), phosphonoacetate, phosphorodithioate, methylphosphonate (MP), sulfonylphosphoramidate, PNms, or PNdmi internucleotide linkage.

7. An EON according to claim 8 or 9, wherein the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl;-O-alkyl-O-alkyl;-methoxy;-aminopropoxy;-methoxyethoxy;-dimethylamino oxyethoxy; and -dimethylaminoethoxyethoxy.

8. An EON according to any one of claims 1 to 7, wherein the EON is 29, 30, 31, or 32 nucleotides in length, wherein the orphan nucleotide is nucleotide position 0, wherein the EON comprises 5, 6, or 7 nucleotides at the 3′ side of the orphan nucleotide, and wherein the EON comprises 22, 23, or 24 nucleotides at the 5′ side of the orphan nucleotide.

9. An EON according to any one of claims 1 to 8, wherein the EON has the structure:
5′-N₂₄N₂₃N₂₂N₂₁N₂₀N₁₉N₁₈N₁₇N₁₆N₁₅N₁₄N₁₃N₁₂N₁₁N₁₀N₉N₈N₇N₆N₅N₄N₃
N₂N₁^{θ}ZdAd^{∧}M₂M₃M₄M₅M₆M₇-3′
wherein:
Zd is the orphan nucleotide at nucleotide position 0, which is a deoxynucleotide carrying a Benner's base;
N₁ is a thymidine (5-methyluridine with a deoxyribose; m5Ud), or a 5-methyluridine with a 2′-MOE ribose substitution (m5Ue);
N₂ is a cytidine with a 2′-fluoro (Cf) or a 2′-OMe (Cm) ribose substitution;
N₃ is a uridine with a 2′-fluoro (Uf) or a 2′-OMe (Um) ribose substitution;
N₄ is a cytidine with a 2′-fluoro ribose substitution (Cf), or a guanosine with a 2′-fluoro (Gf) or a 2′-OMe (Gm) ribose substitution;
N₅ is a guanosine with a 2′-fluoro ribose substitution (Gf), or a cytidine with a 2′-fluoro (Cf) or a 2′-OMe (Cm) ribose substitution;
N₆, N₇, N₁₃, N₁₄, N₁₅, N₁₈, N₁₉, N₂₂, and M₂ are uridine with either a 2′-fluoro ribose substitution (Uf) or a 2′-OMe ribose substitution (Um);
N₈, N₉, N₁₀, and N₂₁ are cytidine with either a 2′-fluoro ribose substitution (Cf) or a 2′-OMe ribose substitution (Cm);
N₁₁, N₁₂, and M₃ are guanosine with either a 2′-fluoro ribose substitution (Gf) or a 2′-OMe ribose substitution (Gm);
M₄ is guanosine with a 2′-fluoro ribose substitution (Gf), a 2′-OMe ribose substitution (Gm), or, preferably, a 2′-MOE ribose substitution (Ge);
N₁₆, N₁₇, N₂₀, and M₅ are adenosine with either a 2′-fluoro ribose substitution (Af) or a 2′-OMe ribose substitution (Am);
N₂₃ and N₂₄ are absent; or N₂₃ is Gf or Gm, and N₂₄ is absent; or N₂₃ is Gf or Gm, and N₂₄ is Um;
Me and M₇ are absent; or Me is Cm and M₇ is absent; or Me is Cm and M₇ is Am or Af;
Ad is deoxyadenosine;
θ is at linkage position 0, and is a PO linkage;
\hat{ } is at linkage position −2 and is an MP or a PNms linkage;
linkage positions −3 and −5 are, each independently, a PO or a PS linkage, preferably a PO linkage;
linkage position +23 is a PNdmi or a PNms linkage;
linkage position −7 is a PNdmi or a PNms linkage; and
all other linkages are PS linkages.

10. An EON according to any one of claims 1 to 8, wherein the EON is selected from the group consisting of SEQ ID NO: 1010, 1013, 1011, 1012, 1014, 25, 217, 372, 580, 559, 506, 534, 595, 106, 412, 189, 767, 345, 465, 535, 677, 184, 627, 95, 363, 805, 957, 279, 277, 367, 96, 523, 956, 1037, 1033, 1034, 1019, 27, 28, and 29.

11. An EON according to any one of claims 1 to 10, wherein:
the EON is bound to a GalNAc moiety, preferably a tri-antennary GalNAc moiety, to improve delivery to hepatocytes;
the EON is packaged into a lipid nanoparticle (LNP) to improve in vivo delivery; or
wherein the EON is conjugated to saponin AG1856 to increase endosomal release and/or intracellular trafficking.

12. A vector, preferably a viral vector, more preferably an adeno-associated virus (AAV) vector, comprising a nucleic acid molecule encoding an EON according to any one of claims 1 to 4.

13. A pharmaceutical composition comprising an EON according to any one of claims 1 to 11, or a vector according to claim 12, and a pharmaceutically acceptable carrier.

14. An EON according to claims 1 to 11, for use in the treatment, amelioration, or prevention of a disorder caused by elevated plasma concentrations of Low-Density Lipoprotein Cholesterol (LDL-C) and/or triglycerides, such as atherosclerotic cardiovascular disease (ASCVD).

15. Use of an EON according to any one of claims 1 to 11, in the manufacture of a medicament for the treatment, amelioration, or prevention of a disorder caused by elevated plasma concentrations of LDL-C and/or triglycerides, such as ASCVD.

16. A method of editing an ANGPTL3 polynucleotide, the method comprising contacting the ANGPTL3 polynucleotide with an EON capable of effecting an ADAR-mediated adenosine to inosine alteration of an adenosine in a codon encoding an amino acid residue associated with heparin binding, thereby editing the ANGPTL3 polynucleotide.

17. A method of treating, ameliorating, or preventing a disorder caused by elevated plasma concentrations of LDL-C and/or triglycerides, such as ASCVD, in a patient in need thereof, the method comprising administering to a patient in need thereof a therapeutically effective amount of an EON according to any one of claims 1 to 11.

18. A method for the deamination of a target adenosine in an ANGPTL3 pre-mRNA or mRNA molecule in a cell, the method comprising the steps of:
(i) providing the cell with an EON according to any one of claims 1 to 11;
(ii) allowing uptake by the cell of the EON;
(iii) allowing annealing of the EON to the ANGPTL3 pre-mRNA or mRNA molecule; and
(iv) allowing an endogenous ADAR enzyme to deaminate the target adenosine in the target RNA molecule to an inosine.

19. A method according to claim 18, further comprising step (v) identifying the presence of the inosine in the target RNA molecule, preferably wherein step (v) comprises:
a) determining the sequence of the ANGPTL3 pre-mRNA or mRNA molecule;
b) assessing the presence of an ANGPTL3 protein with a diminished or absent functionality in lipase inhibition; or
c) using a functional read-out, such as a biomarker read-out, preferably assessing a level of LDL-C and/or triglycerides in a plasma sample and compare it to the levels before providing the EON, wherein a decrease in LDL-C and/or triglyceride levels is indicative for the occurrence of an ANGPTL3 protein with a diminished or absent functionality in lipase inhibition.

20. A method according to any one of claims 16 to 19, wherein the target adenosine is the first nucleotide in the codon encoding lysine at position 63 in the human ANGPTL3 protein.
